(19) United States
(12) Patent Application Publication
(10) Pub. No.: US 2014/0363469 A1
(43) Pub. Date: Dec. 11, 2014

(54) VIRAL ATTENUATION AND VACCINE PRODUCTION

(71) Applicant: Alnylam Pharmaceuticals, Inc., Cambridge, MA (US)

(72) Inventors: Rachel Meyers, Cambridge, MA (US); Brian Bettencourt, Cambridge, MA (US); Jamie Evan Wong, Cambridge, MA (US); John M. Maraganore, Cambridge, MA (US)

(21) Appl. No.: 14/373,195

(22) PCT Filed: Jan. 16, 2013
(86) PCT No.: PCT/US2013/021704

§ 371 Date: Jul. 18, 2014

Related U.S. Application Data
(60) Provisional application No. 61/588,309, filed on Jan. 19, 2012.

(51) Int. Cl. A61K 39/245; C12N 7/00

(52) U.S. Cl. CPC A61K 39/245; C12N 7/00; C12N 2710/16634; C12N 2710/16662
USPC 424/231.1; 435/236

(57) ABSTRACT

The present invention is directed to the generation of attenuated viruses or viral transcripts for the production of vaccines by incorporating microRNA binding sites within the viral target sequence of the pathogen.

### CROSS REFERENCE TO RELATED APPLICATIONS

This application claims the benefit of U.S. Provisional Application No. 61/588,309, filed Jan. 19, 2012 entitled “VIRAL ATTENUATION AND VACCINE PRODUCTION”, the contents of which is incorporated by reference in its entirety.

### REFERENCE TO SEQUENCE LISTING

The present application is being filed along with a Sequence Listing in electronic format. The Sequence Listing is provided as a file entitled ALN168WOSEQLST.txt created on Jan. 16, 2013 which is 1,070,041 bytes in size. The information in electronic format of the sequence listing is incorporated herein by reference in its entirety.

### FIELD OF THE INVENTION

The present invention is directed to the generation of attenuated viruses or viral transcripts for the production of vaccines.

### BACKGROUND OF THE INVENTION

Herpes simplex virus type 1 (HSV-1; HHV1) and Herpes simplex virus type 2 (HSV-2; HHV2) are common human pathogens which cause a variety of clinical illnesses, including oral-facial infections, genital herpes, ocular infections, herpes encephalitis, and neonatal herpes.

The Herpes simplex virus has a rapid lytic replication cycle and the ability to invade sensory neurons where highly restricted gene expression occurs during a latent or nonpathologic state. Such latent infections are subject to reactivation whereby infectious virus can be recovered in peripheral tissue enervated by the latently infected neurons following a specific physiological stress. A major factor in the switch from lytic to latent infection and back involves changes in transcription patterns, mainly as a result of the interaction between viral promoters, the viral genome, and cellular transcriptional machinery. The ability to interfere with any of these pathways could prove useful in the development of vaccines against the family of viruses.

To this end, efforts to effectively attenuate the HSV virus have met with significant challenges. The Herpes genome is quite large and complex. The genome of the Herpes virus is a nuclear replicating, double-stranded DNA approximately 152,000 base pairs in length which circularizes upon infection and which encodes some 100-200 genes. These genes encode a variety of proteins involved in forming the capsid, tegument and envelope of the virus, as well as controlling the replication and infectivity of the virus. The HSV envelope alone contains at least 8 glycoproteins while the matrix or tegument which contacts both the envelope and the capsid contains at least 15-20 proteins. Consequently, approaches to design an effective vaccine against HSV have been unsuccessful to date.

The present invention solves the problem in the art through the use of engineered viral transcripts (in whole or in part) incorporating one or more microRNA (miRNA) target or binding sites.

### SUMMARY OF THE INVENTION

Described herein are compositions and methods useful in the control, regulation, exploitation and study of viral transcripts, particularly those in the Herpesviridae family. Also described are compositions and methods for the diagnosis, prevention, amelioration and/or treatment of viral infections involving the replication status or activity of viruses, particularly Herpes viruses.

The present invention embraces, in one embodiment, a mutant HSV-1 strain comprising at least one miRNA site such as for example those listed in Table 3. The mutant HSV-1 strain may include one or more miRNA sites, is present in a translated or untranslated region of an HSV-1 gene encoded by the HSV-1 strain. In one embodiment, the untranslated region may be selected from the group consisting of the 3′UTR, the 5′ UTR, an intron, and an intragenic region. The miRNA sites may range in size from 17-25, or longer. They may also be subportions as small as 6 nucleotides in length. Where multiple miRNA sites are engineered into the viral target sequence, they may have the same or different sequences. There may be a plurality of miRNA sites, e.g., 2 or more, 3 or more or 5 or more. Further to the invention are methods of immunizing a subject with an HSV-1 antigen comprising contacting said subject with a composition comprising a mutant HSV-1 strain, mutant HSV-1 gene or mutant HSV-1 polynucleotide sequence, wherein the mutant strain, gene or polynucleotide sequence has been engineered to contain at least one miRNA site of Table 3. Administration may be more than once and may occur on an immunization or booster schedule. The composition administered as a vaccine may be formulated for systemic delivery and the formulation may comprise saline or include carriers and/or excipients. The vaccines may also be delivered with adjuvants such as lipids or lipid-like molecules.

The details of various embodiments of the invention are set forth in the description below. Other features, objects, and advantages of the invention will be apparent from the description and the drawings, and from the claims.

### BRIEF DESCRIPTION OF THE FIGURES

FIG. 1 is a schematic showing alternatives to engineering attenuated viruses by incorporating miRNA sites into the 5′UTR, CDS or 3′UTR of a viral transcript. Shown in FIG. 1A is the incorporation into the wild-type (wt) US1 gene of HSV-1 of mir-128, 135a and 183 sites to produce mutant (mt) sequences. Shown in FIG. 1B is the incorporation into the HSV-1 RL2 gene of mir-124 and mir-9 sites. In the figure, “nonessential” indicates that the first position of the miRNA-target pair is not essential for activity. “Silent” refers to a silent substitution, “Cons” means conservative replacement substitution; “Noncons” means nonconservative replacement substitution where “replacement” means changing the amino acid encoded by the codon containing that nucleotide.

### DETAILED DESCRIPTION OF THE INVENTION

The present invention is directed to the design, generation, and production of useful vaccines through attenuation or modification of wild-type viral sequences in order to elicit from a patient or subject an immune response sufficient to ensure protection against an insult from the pathogen in the future. In presently doing so, viral attenuation is achieved through the utilization of microRNA (miRNA) sequences (including miRNA seeds), sites and signatures.

Specifically, it has been discovered that incorporation of one or more miRNA sequences, seeds or signatures into an HSV viral target sequence can lead to post-infection or host-supported viral attenuation. This occurs because the presence of the incorporated miRNA site within the viral sequence elicits binding by endogenous microRNAs present in the cells or tissue. This binding may interfere with critical replication pathways and results in an attenuated virus which, by definition, may now function as a vaccine. As used herein, the term “miRNA site” refers to a nucleotide sequence to which a microRNA binds or associates. It should be understood that “binding” may follow traditional Watson-Crick hybridization rules or may reflect any stable association of the microRNA with the viral target sequence at or adjacent to the miRNA site.

For example, a mutant HSV strain, which is engineered to contain one or more miRNA sites (a region of nucleic acid sequence to which a miRNA will bind) would, upon entering a cell, such as an epithelial cell, be susceptible to binding by any microRNAs present which recognize the engineered site. Upon binding, viral replication or other critical viral lifecycle processes would be compromised thereby reducing or eliminating the threat of viral infection but providing a sufficient trigger for the host organism to mount an immune response.

According to the present invention, the virus which is the target of the vaccine will be one that is capable of infecting eukaryotic cells, e.g., mammalian cells, avian cells, murine cells, human cells and the like. In various embodiments, the virus belongs to the Herpesviridae, Retroviridae, Reoviridae, Adenoviridae, Flaviviridae, Poxyiridae, Caliciviridae, Togaviridae, Coronaviridae, Rhabdoviridae, Filoviridae, Paramyxoviridae, Orthomyxoviridae, Bunyaviridae, Arenaviridae, Bornaviridae, Polyomaviridae, Papillomaviridae, Parvoviridae, Hepadnaviridae or Picornaviridae families.

In one embodiment, the virus is selected from Adenovirus, Cytomegalovirus (e.g., HCMV, HHV5), Epstein Barr virus (e.g., EBV, HHV4), Human Papilloma virus (HPV), MHV-68, Human Immunodeficiency Virus (HIV), Hepatitis A Virus (HAV), Hepatitis B Virus (HBV), Hepatitis C Virus (HCV), Hepatitis E Virus (HEV), Rubella Virus, Mumps Virus, Measles Virus, Respiratory Syncytial Virus, Human T-cell Leukemia Virus, Lentivirus, Herpes Simplex Virus (e.g., Herpes Simplex 1 (HSV1, HHV1), Herpes Simplex 2 (HSV2, HHV2)), Varicella-Zoster Virus (e.g., HHV3), Human Herpesviruses 6A, 6B, and 7, Kaposi's Sarcoma-Associated Herpesvirus (e.g., KSHV, HHV8), Cercopithecine Herpesvirus, Hepatitis Delta Virus, Dengue Virus, Foot and Mouth Disease Virus, Polyomavirus (e.g., JC, BK), Poliovirus, Coxsackievirus, Echovirus, Rhinovirus, Vacciniavirus, Small Pox Virus, Influenza Virus, or Avian Influenza Virus.

In particular, the virus belongs to the Herpesviridae family and is selected from the alpha viruses (HHV1, HHV2 or HHV3), the beta viruses (HHV5, 6A, 6B or HHV7) or the gamma viruses (HHV8 or HHV4).

According to the present invention wild-type viral sequences are engineered to contain one or more miRNA sequences, sites or signatures thus producing a mutant viral sequence. A “viral sequence” or “viral target sequence” includes any polynucleotide (DNA or RNA or combination thereof) which is viral in origin. As used herein, “wild-type” means that state, status or type which is naturally found in nature. “Mutant (mt)” sequences are those which have been altered in some form whether by insertion, deletion, duplication, inversion or the like and which differ from the wild-type version of the sequence.

The wild-type viral target sequences to be engineered include genomic sequences (in whole or in part), gene sequences, or subregions or features of these sequences such as repeat regions, inverted regions, polyA tails, coding regions, promoters, 5′ or 3′ untranslated regions (UTRs), intronic regions, or any intervening viral sequence or subportion thereof.

Shown in Table 1 are representative examples of viral targets of the present invention. Listed in Table 2 are the 77 genes of the HSV-1 genome. Given are the nucleotide ranges of SEQ ID NO: 1 that define each of the genes. Where the range is preceded by the term “Complement” it is to be understood that the particular gene is encoded on the opposite strand of the dsDNA virus and hence the sequence represents the complement of the nucleotide range given. Also listed is a description of the type of protein encoded by each gene.

TABLE 1

| | Viral Transcript Reference | |
|---|---|---|
| Virus Name | Sequence (genome) | SEQ ID |
| HSV-1 | NC_001806.1 | 1 |
| HSV-2 | NC_001798.1 | 2 |

TABLE 2

| | | Nucleotide range of | |
|---|---|---|---|
| | HSV-1 | NC_001806.1 | |
| | Gene | (SEQ ID 1) | Protein Product |
| 1 | RL1 | 513-1539 | neurovirulence protein ICP34.5 |
| 2 | RL2 | 2086-5698 | Ubiquitin E3 ligase ICP0 |
| 3 | UL1 | 9337-10948 | Envelope glycoprotein L |
| 4 | UL2 | 9884-10948 | Uracyl-DNA glycosylase |
| 5 | UL3 | 10957-11720 | Nuclear protein UL3 |
| 6 | UL4 | Complement (11753-12422) | Nuclear protein UL4 |
| 7 | UL5 | Complement (11753-15131) | helicase-primase helicase subunit |
| 8 | UL6 | 15130-18040 | Capsid portal protein |
| 9 | UL7 | 17135-18040 | Tegument protein UL7 |
| 10 | UL8 | Complement (18210-20476) | helicase-primase subunit |
| 11 | UL9 | Complement (18210-23259) | DNA replication origin-binding helicase |
| 12 | UL10 | 23204-24648 | envelope glycoprotein M |
| 13 | UL11 | Complement (24800-25501) | myristylated tegument protein |
| 14 | UL12 | Complement (24800-27046) | deoxyribonuclease |
| 15 | UL13 | Complement (24800-28691) | tegument serine/threonine protein kinase |
| 16 | UL14 | Complement (24800-28915) | tegument protein UL14 |
| 17 | UL15 | 28804-34825 | DNA packaging terminase subunit 1 |
| 18 | UL16 | Complement (30173-31670) | tegument protein UL16 |
| 19 | UL17 | Complement (30173-33666) | DNA packaging tegument protein UL17 |
| 20 | UL18 | Complement (35023-36051) | Capsid triplex subunit 2 |
| 21 | UL19 | Complement (35023-40768) | Major capsid protein |
| 22 | UL20 | Complement (35023-41488) | Envelope protein UL20 |
| 23 | UL21 | 42074-43695 | Tegument protein UL21 |
| 24 | UL22 | Complement (43824-46581) | Envelope glycoprotein H |
| 25 | UL23 | Complement (46608-47911) | Thymidine kinase |
| 26 | UL24 | 47737-48744 | Nuclear protein UL24 |
| 27 | UL25 | 48813-52771 | DNA packaging tegument protein UL25 |
| 28 | UL26 | 50809-52771 | Capsid maturation protease |
| 29 | UL26.5 | 51727-52771 | Capsid scaffold protein |
| 30 | UL27 | Complement (53058-56080) | Envelope glycoprotein B |
| 31 | UL28 | Complement (53058-58320) | DNA packaging terminase subunit 2 |
| 32 | UL29 | Complement (58409-62053) | Single-stranded DNA-binding protein |
| 33 | UL30 | 62606-66553 | DNA polymerase catalytic subunit |
| 34 | UL31 | Complement (66377-67379) | Nuclear egress lamina protein |
| 35 | UL32 | Complement (66377-69162) | DNA packaging protein UL32 |
| 36 | UL33 | 69161-70943 | DNA packaging protein UL33 |
| 37 | UL34 | 69633-70943 | Nuclear egress membrane protein |
| 38 | UL35 | 70566-70943 | Small capsid protein |
| 39 | UL36 | Complement (70983-80543) | Large tegument protein |
| 40 | UL37 | Complement (80712-84084) | Tegument protein UL37 |
| 41 | UL38 | 84531-86021 | Capsid triplex subunit 1 |
| 42 | UL39 | 86217-90988 | Ribonucleotide reductase subunit 1 |
| 43 | UL40 | 89773-90988 | Ribonucleotide reductase subunit 2 |
| 44 | UL41 | Complement (91116-92740) | Tegument host shutoff protein |
| 45 | UL42 | 92920-94638 | DNA polymerase processivity subunit |
| 46 | UL43 | 94748-96068 | Envelope protein UL43 |
| 47 | UL44 | 96168-98998 | Envelope glycoprotein C |
| 48 | UL45 | 97953-98668 | Membrane protein UL45 |
| 49 | UL46 | Complement (98726-100998) | Tegument protein VP11/12 |
| 50 | UL47 | Complement (98726-103116) | Tegument protein VP13/14 |
| 51 | UL48 | Complement (103537-105259) | Transactivating tegument protein VP16 |
| 52 | UL49A | Complement (105462-106993) | Envelope glycoprotein N |
| 53 | UL49 | Complement (105462-106391) | Tegument protein VP22 |
| 54 | UL50 | 107010-108157 | Deoxyuridine triphosphatase |
| 55 | UL51 | Complement (108276-109011) | Tegument protein UL51 |
| 56 | UL52 | 109048-113448 | Helicase-primase primase subunit |
| 57 | UL53 | 112179-113448 | Envelope glycoprotein K |
| 58 | UL54 | 113596-115282 | Multifunctional expression regulator |
| 59 | UL55 | 115496-116103 | Nuclear protein UL55 |
| 60 | UL56 | Complement (116196-116925) | membrane protein UL56 |
| 61 | RL2 | Complement (120673-124285) | Ubiquitin E3 ligase ICP0 |
| 62 | RL1 | Complement (124832-125858) | Neurovirulence protein ICP34.5 |
| 63 | RS1 | Complement (127170-131457) | Transcriptional regulator ICP4 |
| 64 | US1 | 132098-133960 | Regulator protein ICP22 |
| 65 | US2 | Complement (134023-135333) | Virion protein US2 |
| 66 | US3 | 134934-137531 | Serine/threonine protein kinase US3 |
| 67 | US4 | 136702-137531 | Envelope glycoprotein G |
| 68 | US5 | 137596-141048 | Envelope glycoprotein J |
| 69 | US6 | 138309-141048 | Envelope glycoprotein D |
| 70 | US7 | 139668-141048 | Envelope glycoprotein I |
| 71 | US8 | 141139-143693 | Envelope glycoprotein E |
| 72 | US8A | 142744-143693 | Membrane protein US8A |
| 73 | US9 | 143219-143693 | Membrane protein US9 |
| 74 | US10 | Complement (144119-145194) | Virion protein US10 |
| 75 | US11 | Complement (144119-145490) | Tegument protein US11 |
| 76 | US12 | Complement (144119-146135) | TAP transporter inhibitor ICP47 |
| 77 | RS1 | 146776-151063 | Transcriptional regulator ICP4 |

Of the 77 genes in the HSV-1 genome, certain genes are more likely targets for attenuation. These include, the essential DNA replication HSV proteins: UL9, UL29, UL5, UL52, ULB, UL30, UL42; the immediate early genes: ICPO, ICP4, ICP27, ICP22; and the immune evasion genes: ICP47, and UL4.

Viral attenuation for the production of a vaccine may be achieved in one of several ways. For example, incorporation of one or more miRNA sites or signatures into a wild-type viral target sequence and then administration of the mutant viral strain may result in attenuation. As used herein “attenuation” means the process by which an infectious agent is altered in whole or part so that it becomes harmless or less virulent. An attenuated virus may serve as a vaccine. It is also understood that a portion, gene, or region of the viral target sequences comprising one or more miRNA sites described here may serve as a vaccine. A “vaccine” is any composition, compound or molecule that improves immunity to a particular disease. Vaccines of the present invention may be used to stimulate the production of antibodies and provide immunity against one or more diseases, viral and the like. In some cases, a vaccine resembles a disease-causing microorganism such as a virus, and is often made from weakened or killed forms of the virus, its toxins or one of its proteins. Vaccines of the present invention may be polynucleotides, polypeptides or combinations of both, e.g., chimeric molecules. They may be bound or associated with non-nucleic acid or non-protein moieties or conjugates. Vaccines of the present invention may comprise an entire viral genome which has been mutated by the addition of one miRNA site which shares some homology to the insertion point and they may also comprise the viral genome which has had inserted therein multiple sites. These multiple sites may be incorporated into one viral region or feature, e.g., a 3′UTR, or may be inserted across multiple features of the viral genome. Further, the present invention is not limited to the insertion or engineering of only one miRNA site (one miRNA sequences' complement) per viral target sequence. Multiple different miRNA may be used as the source of sites to be inserted. Likewise, the exact site sequence need not be used. Sites inserted may be 100% identical to the wild-type miRNA site. They may also be at least 90%, at least 80%, at least 70%, at least 60%, at least 50%, at least 40%, at least 30% or at least 20% identical. It will be understood that the percent identity may be higher where shorter mature miRNA sites or miRNA seed sites are used.

Fusion molecules are also contemplated by the invention. Fusion of the viral genome, gene, or target sequence to one or more nucleic acids or proteins is contemplated. For research purposes, it will be useful to fuse one or more viral target sequences (whether wild type or mutant) to a reporter molecule such as luciferase. Dual reporters may also be used and may be fluorescent, colorimetric, etc.

In one embodiment the viral target sequence of the vaccine will be of Herpes virus origin. In one embodiment the viral target sequence will be derived from the HSV-1 genome (SEQ ID NO:1). Where the vaccines of the present invention are nucleic acid based, they will comprise at least one miRNA binding or target site.

The viral target sequence of the vaccines of the present invention may comprise the entire HSV genome with one or more added miRNA binding sites or may be a portion of the HSV genome. As used herein, an miRNA “binding site” refers to a sequence that may foster interaction of an miRNA and the sequence. This interaction need not be complete binding as that term is known in the art and may be less than 100 percent hybridization. A binding site may also be referred to as a “target site”. Mismatches as between the sequence of any endogenous miRNA and the binding or targeting site engineered into the viral target sequence is contemplated as part of the invention.

In one embodiment, the vaccine is an HSV mutant strain DNA polynucleotide which is 152,261 nucleotides in length and comprises one or more miRNA binding sites engineered into the wild type genome to produce the mutant strain.

In one embodiment the vaccine of the invention is between 100,000-200,000 nucleotides in length. The vaccine may be composed of only one of the genes of the virus which has incorporated or engineered therein, one or more miRNA binding sites. In this embodiment the vaccine sequence may be from 100 to 100,000, from 500 to 50,000, from 1,000 to 5,000 nucleotides in length. It is to be understood that where the virus is a double stranded virus (whether DNA or RNA), the lengths recited or listed ranges may refer to the number of base pairs present in the vaccine.

Mammalian genomes are predicted to encode at least 200 to 1000 distinct miRNAs, many of which are estimated to interact with 5-10 different mRNA transcripts. Accordingly, miRNAs are predicted to regulate most if not all genes. miRNAs are differentially expressed in various tissues, such that each tissue is characterized by a specific set of miRNAs. miRNAs have been shown to be important modulators of cellular pathways including growth and proliferation, apoptosis, and developmental timing.

In the context of the present invention, miRNA sequences, including their pre-, pri- and mature sequences, as well as miRNA seeds and signatures may be used to design miRNA sites which are added to wild type viral target sequences in order to produce the vaccine compositions of the present invention.

The miRNA sequences (including miRNA seeds, sites, signatures and/or precursors) which may be incorporated into the wild type viral target sequences may be from any known miRNA such as those taught in US Publication US2005/0261218 and US Publication US2005/0059005, the contents of which are incorporated herein by reference in their entirety.

The miRNA sites of the present invention may encompass “miRNA precursors” or “mature miRNA” or variants or “miRNA seeds”, or combinations thereof. A miRNA “seed” is that sequence with nucleotide identity at positions 2-8 of the mature miRNA. In one embodiment, a miRNA seed comprises positions 2-7 of the mature miRNA. In another embodiment, a miRNA seed may comprise 8 nucleotides (e.g., nucleotides 2-8 of the mature miRNA) having an adenine (A) at position 1. In another embodiment, a miRNA seed may comprise 7 nucleotides (e.g., nucleotides 2-7 of the mature miRNA) having an adenine (A) at position 1. See for example, Grimson A, Farh K K, Johnston W K, Garrett-Engele P, Lim L P, Bartel D P; Mol. Cell. 2007 Jul. 6; 27(1):91-105.

As used herein, the term “miRNA precursor” is used to encompass, without limitation, primary RNA transcripts, pri-miRNAs and pre-miRNAs. Examples of small non-coding RNAs include, but are not limited to, primary miRNA transcripts (also known as pri-pre-miRNAs, pri-mirs and pri-miRNAs, which range from around 70 nucleotides to about 450 nucleotides in length and often taking the form of a hairpin structure); pre-miRNAs (also known as pre-mirs and foldback miRNA precursors, which range from around 50 nucleotides to around 110 nucleotides in length); miRNAs (also known as microRNAs, Mirs, miRs, mirs, and mature miRNAs, and generally refer either to intermediate molecules around 17 to about 25 nucleotides in length, or to single-stranded miRNAs, which may comprise a bulged structure upon hybridization with a partially complementary target nucleic acid molecule); or mimics of pri-miRNAs, pre-miRNAs or miRNAs. Examples of each of these types of miRNA constructs is taught in, for example, US Publication US2005/0261218 to Esau et. al, the contents of which are incorporated herein by reference in its entirety.

In some embodiments, the pri-miRNAs which may be incorporated into viral target sequences to create a miRNA binding site are 70 to 450 nucleobases in length. One having ordinary skill in the art will appreciate that this embodies compounds of 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449 or 450 nucleobases in length, or any range therewithin.

In some embodiments, pri-miRNAs, which may be incorporated into viral target sequences to create a miRNA binding site are 110 to 430 nucleobases in length. One having ordinary skill in the art will appreciate that this embodies compounds of 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429 or 430 nucleobases in length, or any range therewithin.

In some embodiments, pri-miRNAs, which may be incorporated into viral target sequences to create a miRNA binding site are 110 to 280 nucleobases in length. One having ordinary skill in the art will appreciate that this embodies compounds of 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279 or 280 nucleobases in length, or any range therewithin.

In some embodiments, pre-miRNAs, which may be incorporated into viral target sequences to create a miRNA binding site are 50 to 110 nucleobases in length. One having ordinary skill in the art will appreciate that this embodies compounds of 50, 51, 52, 53, 54, 55, 56, 57, 58, 59, 60, 61, 62, 63, 64, 65, 66, 67, 68, 69 70, 71 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 or 110 nucleobases in length, or any range therewithin.

In some embodiments, pre-miRNAs, which may be incorporated into viral target sequences to create a miRNA binding site are 60 to 80 nucleobases in length. One having ordinary skill in the art will appreciate that this embodies compounds of 60, 61, 62, 63, 64, 65, 66, 67, 68, 69, 70, 71, 72, 73, 74, 75, 76, 77, 78, 79, or 80 nucleobases in length, or any range therewithin.

In some embodiments, miRNAs, which may be incorporated into viral target sequences to create a miRNA binding site are 15 to 49 nucleobases in length. One having ordinary skill in the art will appreciate that this embodies compounds of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 nucleobases in length, or any range therewithin.

In some embodiments, miRNAs, which may be incorporated into viral target sequences to create a miRNA binding site are 17 to 25 nucleobases in length. One having ordinary skill in the art will appreciate that this embodies compounds of 17, 18, 19, 20, 21, 22, 23, 24 or 25 nucleobases in length, or any range therewithin.

miRNA of human origin are of particular use in the present invention. These microRNAs, as well as their reverse complements (or sites) are listed in Table 3 below.

TABLE 3
Homo sapiens miRNA

| | | SEQ | | SEQ |
|---|---|---|---|---|
| | | ID | REVERSE COMPLMENT | ID |
| miRNA Name | miRNA (5′-3′) | NO: | (miRNA site) | NO: |
| let-7a-2-3p | CUGUACAGCCUCCUAGC | 3 | GGAAAGCUAGGAGGCUG | 4 |
| | UUUCC | | UACAG | |
| let-7a-3p | CUAUACAAUCUACUGUC | 5 | GAAAGACAGUAGAUUGU | 6 |
| | UUUC | | AUAG | |
| let-7a-5p | UGAGGUAGUAGGUUGUA | 7 | AACUAUACAACCUACUAC | 8 |
| | UAGUU | | CUCA | |
| let-7b-3p | CUAUACAACCUACUGCC | 9 | GGGAAGGCAGUAGGUUG | 10 |
| | UUCCC | | UAUAG | |
| let-7b-5p | UGAGGUAGUAGGUUGUG | 11 | AACCACACAACCUACUAC | 12 |
| | UGGUU | | CUCA | |
| let-7c | UGAGGUAGUAGGUUGUA | 13 | AACCAUACAACCUACUAC | 14 |
| | UGGUU | | CUCA | |
| let-7d-3p | CUAUACGACCUGCUGCC | 15 | AGAAAGGCAGCAGGUCGU | 16 |
| | UUUCU | | AUAG | |
| let-7d-5p | AGAGGUAGUAGGUUGCA | 17 | AACUAUGCAACCUACUAC | 18 |
| | UAGUU | | CUCU | |
| let-7e-3p | CUAUACGGCCUCCUAGC | 19 | GGAAAGCUAGGAGGCCGU | 20 |
| | UUUCC | | AUAG | |
| let-7e-5p | UGAGGUAGGAGGUUGUA | 21 | AACUAUACAACCUCCUAC | 22 |
| | UAGUU | | CUCA | |
| let-7f-1-3p | CUAUACAAUCUAUUGCC | 23 | GGGAAGGCAAUAGAUUG | 24 |
| | UUCCC | | UAUAG | |
| let-7f-2-3p | CUAUACAGUCUACUGUC | 25 | GGAAAGACAGUAGACUG | 26 |
| | UUUCC | | UAUAG | |
| let-7f-5p | UGAGGUAGUAGAUUGUA | 27 | AACUAUACAAUCUACUAC | 28 |
| | UAGUU | | CUCA | |
| let-7g-3p | CUGUACAGGCCACUGCC | 29 | GCAAGGCAGUGGCCUGUA | 30 |
| | UUGC | | CAG | |
| let-7g-5p | UGAGGUAGUAGUUUGUA | 31 | AACUGUACAAACUACUAC | 32 |
| | CAGUU | | CUCA | |
| let-7i-3p | CUGCGCAAGCUACUGCC | 33 | AGCAAGGCAGUAGCUUGC | 34 |
| | UUGCU | | GCAG | |
| let-7i-5p | UGAGGUAGUAGUUUGUG | 35 | AACAGCACAAACUACUAC | 36 |
| | CUGUU | | CUCA | |
| miR-1 | UGGAAUGUAAAGAAGUA | 37 | AUACAUACUUCUUUACAU | 38 |
| | UGUAU | | UCCA | |
| miR-100-3p | CAAGCUUGUAUCUAUAG | 39 | CAUACCUAUAGAUACAAG | 40 |
| | GUAUG | | CUUG | |
| miR-100-5p | AACCCGUAGAUCCGAAC | 41 | CACAAGUUCGGAUCUACG | 42 |
| | UUGUG | | GGUU | |
| miR-101-3p | UACAGUACUGUGAUAAC | 43 | UUCAGUUAUCACAGUACU | 44 |
| | UGAA | | GUA | |
| miR-101-5p | CAGUUAUCACAGUGCUG | 45 | AGCAUCAGCACUGUGAUA | 46 |
| | AUGCU | | ACUG | |
| miR-103a-2-5p | AGCUUCUUUACAGUGCU | 47 | CAAGGCAGCACUGUAAAG | 48 |
| | GCCUUG | | AAGCU | |
| miR-103a-3p | AGCAGCAUUGUACAGGG | 49 | UCAUAGCCCUGUACAAUG | 50 |
| | CUAUGA | | CUGCU | |
| miR-103b | UCAUAGCCCUGUACAAU | 51 | AGCAGCAUUGUACAGGGC | 52 |
| | GCUGCU | | UAUGA | |
| miR-105-3p | ACGGAUGUUUGAGCAUG | 53 | UAGCACAUGCUCAAACAU | 54 |
| | UGCUA | | CCGU | |
| miR-105-5p | UCAAAUGCUCAGACUCC | 55 | ACCACAGGAGUCUGAGCA | 56 |
| | UGUGGU | | UUUGA | |
| miR-106a-3p | CUGCAAUGUAAGCACUU | 57 | GUAAGAAGUGCUUACAU | 58 |
| | CUUAC | | UGCAG | |
| miR-106a-5p | AAAAGUGCUUACAGUGC | 59 | CUACCUGCACUGUAAGCA | 60 |
| | AGGUAG | | CUUUU | |
| miR-106b-3p | CCGCACUGUGGGUACUU | 61 | GCAGCAAGUACCCACAGU | 62 |
| | GCUGC | | GCGG | |
| miR-106b-5p | UAAAGUGCUGACAGUGC | 63 | AUCUGCACUGUCAGCACU | 64 |
| | AGAU | | UUA | |
| miR-107 | AGCAGCAUUGUACAGGG | 65 | UGAUAGCCCUGUACAAUG | 66 |
| | CUAUCA | | CUGCU | |
| miR-10a-3p | CAAAUUCGUAUCUAGGG | 67 | UAUUCCCCUAGAUACGAA | 68 |
| | GAAUA | | UUUG | |
| miR-10a-5p | UACCCUGUAGAUCCGAA | 69 | CACAAAUUCGGAUCUACA | 70 |
| | UUUGUG | | GGGUA | |
| miR-10b-3p | ACAGAUUCGAUUCUAGG | 71 | AUUCCCCUAGAAUCGAAU | 72 |
| | GGAAU | | CUGU | |
| miR-10b-5p | UACCCUGUAGAACCGAA | 73 | CACAAAUUCGGUUCUACA | 74 |
| | UUUGUG | | GGGUA | |
| miR-1178 | UUGCUCACUGUUCUUCC | 75 | CUAGGGAAGAACAGUGA | 76 |
| | CUAG | | GCAA | |
| miR-1179 | AAGCAUUCUUUCAUUGG | 77 | CCAACCAAUGAAAGAAUG | 78 |
| | UUGG | | CUU | |
| miR-1180 | UUUCCGGCUCGCGUGGG | 79 | ACACACCCACGCGAGCCG | 80 |
| | UGUGU | | GAAA | |
| miR-1181 | CCGUCGCCGCCACCCGA | 81 | CGGCUCGGGUGGCGGCGA | 82 |
| | GCCG | | CGG | |
| miR-1182 | GAGGGUCUUGGGAGGGA | 83 | GUCACAUCCCUCCCAAGA | 84 |
| | UGUGAC | | CCCUC | |
| miR-1183 | CACUGUAGGUGAUGGUG | 85 | UGCCCACUCUCACCAUCA | 86 |
| | AGAGUGGGCA | | CCUACAGUG | |
| miR-1184 | CCUGCAGCGACUUGAUG | 87 | GGAAGCCAUCAAGUCGCU | 88 |
| | GCUUCC | | GCAGG | |
| miR-1185-1-3p | AUAUACAGGGGGAGACU | 89 | AUAAGAGUCUCCCCCUGU | 90 |
| | CUUAU | | AUAU | |
| miR-1185-2-3p | AUAUACAGGGGGAGACU | 91 | AUGAGAGUCUCCCCCUGU | 92 |
| | CUCAU | | AUAU | |
| miR-1185-5p | AGAGGAUACCCUUUGUA | 93 | AACAUACAAAGGGUAUCC | 94 |
| | UGUU | | UCU | |
| miR-1193 | GGGAUGGUAGACCGGUG | 95 | GCACGUCACCGGUCUACC | 96 |
| | ACGUGC | | AUCCC | |
| miR-1197 | UAGGACACAUGGUCUAC | 97 | AGAAGUAGACCAUGUGUC | 98 |
| | UUCU | | CUA | |
| miR-1200 | CUCCUGAGCCAUUCUGA | 99 | GAGGCUCAGAAUGGCUCA | 100 |
| | GCCUC | | GGAG | |
| miR-1202 | GUGCCAGCUGCAGUGGG | 101 | CUCCCCCACUGCAGCUGG | 102 |
| | GGAG | | CAC | |
| miR-1203 | CCCGGAGCCAGGAUGCA | 103 | GAGCUGCAUCCUGGCUCC | 104 |
| | GCUC | | GGG | |
| miR-1204 | UCGUGGCCUGGUCUCCA | 105 | AUAAUGGAGACCAGGCCA | 106 |
| | UUAU | | CGA | |
| miR-1205 | UCUGCAGGGUUUGCUUU | 107 | CUCAAAGCAAACCCUGCA | 108 |
| | GAG | | GA | |
| miR-1206 | UGUUCAUGUAGAUGUUU | 109 | GCUUAAACAUCUACAUGA | 110 |
| | AAGC | | ACA | |
| miR-1207-3p | UCAGCUGGCCCUCAUUU | 111 | GAAAUGAGGGCCAGCUGA | 112 |
| | C | | | |
| miR-1207-5p | UGGCAGGGAGGCUGGGA | 113 | CCCCUCCCAGCCUCCCUG | 114 |
| | GGGG | | CCA | |
| miR-1208 | UCACUGUUCAGACAGGC | 115 | UCCGCCUGUCUGAACAGU | 116 |
| | GGA | | GA | |
| miR-122-3p | AACGCCAUUAUCACACU | 117 | UAUUUAGUGUGAUAAUG | 118 |
| | AAAUA | | GCGUU | |
| miR-122-5p | UGGAGUGUGACAAUGGU | 119 | CAAACACCAUUGUCACAC | 120 |
| | GUUUG | | UCCA | |
| miR-1224-3p | CCCCACCUCCUCUCUCCU | 121 | CUGAGGAGAGAGGAGGU | 122 |
| | CAG | | GGGG | |
| miR-1224-5p | GGAGGACUCGGGAGGU | 123 | CCACCUCCCGAGUCCUCA | 124 |
| | GG | | C | |
| miR-1225-3p | UGAGCCCCUGUGCCGCC | 125 | CUGGGGGCGGCACAGGGG | 126 |
| | CCCAG | | CUCA | |
| miR-1225-5p | GUGGGUACGGCCCAGUG | 127 | CCCCCCACUGGGCCGUAC | 128 |
| | GGGGG | | CCAC | |
| miR-1226-3p | UCACCAGCCCUGUGUUC | 129 | CUAGGGAACACAGGGCUG | 130 |
| | CCUAG | | GUGA | |
| miR-1226-5p | GUGAGGGCAUGCAGGCC | 131 | CCCCAUCCAGGCCUGCAU | 132 |
| | UGGAUGGGG | | GCCCUCAC | |
| miR-1227 | CGUGCCACCCUUUUCCC | 133 | CUGGGGAAAAGGGUGGC | 134 |
| | CAG | | ACG | |
| miR-1228-3p | UCACACCUGCCUCGCCCC | 135 | GGGGGGCGAGGCAGGUG | 136 |
| | CC | | UGA | |
| miR-1228-5p | GUGGGCGGGGGCAGGUG | 137 | CACACACCUGCCCCCGCC | 138 |
| | UGUG | | CAC | |
| miR-1229 | CUCUCACCACUGCCCUCC | 139 | CUGUGGGAGGGCAGUGG | 140 |
| | CACAG | | UGAGAG | |
| miR-1231 | GUGUCUGGGCGGACAGC | 141 | GCAGCUGUCCGCCCAGAC | 142 |
| | UGC | | AC | |
| miR-1233 | UGAGCCCUGUCCUCCCG | 143 | CUGCGGGAGGACAGGGCU | 144 |
| | CAG | | CA | |
| miR-1234 | UCGGCCUGACCACCCAC | 145 | GUGGGGUGGGUGGUCAG | 146 |
| | CCCAC | | GCCGA | |
| miR-1236 | CCUCUUCCCCUUGUCUC | 147 | CUGGAGAGACAAGGGGA | 148 |
| | UCCAG | | AGAGG | |
| miR-1237 | UCCUUCUGCUCCGUCCC | 149 | CUGGGGGACGGAGCAGAA | 150 |
| | CCAG | | GGA | |
| miR-1238 | CUUCCUCGUCUGUCUGC | 151 | GGGGCAGACAGACGAGGA | 152 |
| | CCC | | AG | |
| miR-124-3p | UAAGGCACGCGGUGAAU | 153 | GGCAUUCACCGCGUGCCU | 154 |
| | GCC | | UA | |
| miR-124-5p | CGUGUUCACAGCGGACC | 155 | AUCAAGGUCCGCUGUGAA | 156 |
| | UUGAU | | CACG | |
| miR-1243 | AACUGGAUCAAUUAUAG | 157 | CACUCCUAUAAUUGAUCC | 158 |
| | GAGUG | | AGUU | |
| miR-1244 | AAGUAGUUGGUUUGUAU | 159 | AACCAUCUCAUACAAACC | 160 |
| | GAGAUGGUU | | AACUACUU | |
| miR-1245a | AAGUGAUCUAAAGGCCU | 161 | AUGUAGGCCUUUAGAUCA | 162 |
| | ACAU | | CUU | |
| miR-1245b-3p | UCAGAUGAUCUAAAGGC | 163 | UAUAGGCCUUUAGAUCAU | 164 |
| | CUAUA | | CUGA | |
| miR-1245b-5p | UAGGCCUUUAGAUCACU | 165 | UUUAAGUGAUCUAAAGG | 166 |
| | UAAA | | CCUA | |
| miR-1246 | AAUGGAUUUUUGGAGCA | 167 | CCUGCUCCAAAAAUCCAU | 168 |
| | GG | | U | |
| miR-1247-3p | CCCCGGGAACGUCGAGA | 169 | GCUCCAGUCUCGACGUUC | 170 |
| | CUGGAGC | | CCGGGG | |
| miR-1247-5p | ACCCGUCCCGUUCGUCC | 171 | UCCGGGGACGAACGGGAC | 172 |
| | CCGGA | | GGGU | |
| miR-1248 | ACCUUCUUGUAUAAGCA | 173 | UUUAGCACAGUGCUUAUA | 174 |
| | CUGUGCUAAA | | CAAGAAGGU | |
| miR-1249 | ACGCCCUUCCCCCCCUUC | 175 | UGAAGAAGGGGGGGAAG | 176 |
| | UUCA | | GGCGU | |
| miR-1250 | ACGGUGCUGGAUGUGGC | 177 | AAAGGCCACAUCCAGCAC | 178 |
| | CUUU | | CGU | |
| miR-1251 | ACUCUAGCUGCCAAAGG | 179 | AGCGCCUUUGGCAGCUAG | 180 |
| | CGCU | | AGU | |
| miR-1252 | AGAAGGAAAUUGAAUUC | 181 | UAAAUGAAUUCAAUUUCC | 182 |
| | AUUUA | | UUCU | |
| miR-1253 | AGAGAAGAAGAUCAGCC | 183 | UGCAGGCUGAUCUUCUUC | 184 |
| | UGCA | | UCU | |
| miR-1254 | AGCCUGGAAGCUGGAGC | 185 | ACUGCAGGCUCCAGCUUC | 186 |
| | CUGCAGU | | CAGGCU | |
| miR-1255a | AGGAUGAGCAAAGAAAG | 187 | AAUCUACUUUCUUUGCUC | 188 |
| | UAGAUU | | AUCCU | |
| miR-1255b-2-3p | AACCACUUUCUUUGCUC | 189 | UGGAUGAGCAAAGAAAG | 190 |
| | AUCCA | | UGGUU | |
| miR-1255b-5p | CGGAUGAGCAAAGAAAG | 191 | AACCACUUUCUUUGCUCA | 192 |
| | UGGUU | | UCCG | |
| miR-1256 | AGGCAUUGACUUCUCAC | 193 | AGCUAGUGAGAAGUCAA | 194 |
| | UAGCU | | UGCCU | |
| miR-1257 | AGUGAAUGAUGGGUUCU | 195 | GGUCAGAACCCAUCAUUC | 196 |
| | GACC | | ACU | |
| miR-1258 | AGUUAGGAUUAGGUCGU | 197 | UUCCACGACCUAAUCCUA | 198 |
| | GGAA | | ACU | |
| miR-125a-3p | ACAGGUGAGGUUCUUGG | 199 | GGCUCCCAAGAACCUCAC | 200 |
| | GAGCC | | CUGU | |
| miR-125a-5p | UCCCUGAGACCCUUUAA | 201 | UCACAGGUUAAAGGGUCU | 202 |
| | CCUGUGA | | CAGGGA | |
| miR-125b-1-3p | ACGGGUUAGGCUCUUGG | 203 | AGCUCCCAAGAGCCUAAC | 204 |
| | GAGCU | | CCGU | |
| miR-125b-2-3p | UCACAAGUCAGGCUCUU | 205 | GUCCCAAGAGCCUGACUU | 206 |
| | GGGAC | | GUGA | |
| miR-125b-5p | UCCCUGAGACCCUAACU | 207 | UCACAAGUUAGGGUCUCA | 208 |
| | UGUGA | | GGGA | |
| miR-126-3p | UCGUACCGUGAGUAAUA | 209 | CGCAUUAUUACUCACGGU | 210 |
| | AUGCG | | ACGA | |
| miR-126-5p | CAUUAUUACUUUUGGUA | 211 | CGCGUACCAAAAGUAAUA | 212 |
| | CGCG | | AUG | |
| miR-1260a | AUCCCACCUCUGCCACC | 213 | UGGUGGCAGAGGUGGGA | 214 |
| | A | | U | |
| miR-1260b | AUCCCACCACUGCCACC | 215 | AUGGUGGCAGUGGUGGG | 216 |
| | AU | | AU | |
| miR-1261 | AUGGAUAAGGCUUUGGC | 217 | AAGCCAAAGCCUUAUCCA | 218 |
| | UU | | U | |
| miR-1262 | AUGGGUGAAUUUGUAGA | 219 | AUCCUUCUACAAAUUCAC | 220 |
| | AGGAU | | CCAU | |
| miR-1263 | AUGGUACCCUGGCAUAC | 221 | ACUCAGUAUGCCAGGGUA | 222 |
| | UGAGU | | CCAU | |
| miR-1264 | CAAGUCUUAUUUGAGCA | 223 | AACAGGUGCUCAAAUAAG | 224 |
| | CCUGUU | | ACUUG | |
| miR-1265 | CAGGAUGUGGUCAAGUG | 225 | AACAACACUUGACCACAU | 226 |
| | UUGUU | | CCUG | |
| miR-1266 | CCUCAGGGCUGUAGAAC | 227 | AGCCCUGUUCUACAGCCC | 228 |
| | AGGGCU | | UGAGG | |
| miR-1267 | CCUGUUGAAGUGUAAUC | 229 | UGGGGAUUACACUUCAAC | 230 |
| | CCCA | | AGG | |
| miR-1268a | CGGGCGUGGUGGUGGGG | 231 | CCCCCACCACCACGCCCG | 232 |
| | G | | | |
| miR-1268b | CGGGCGUGGUGGUGGGG | 233 | CACCCCCACCACCACGCC | 234 |
| | GUG | | CG | |
| miR-1269a | CUGGACUGAGCCGUGCU | 235 | CCAGUAGCACGGCUCAGU | 236 |
| | ACUGG | | CCAG | |
| miR-1269b | CUGGACUGAGCCAUGCU | 237 | CCAGUAGCAUGGCUCAGU | 238 |
| | ACUGG | | CCAG | |
| miR-127-3p | UCGGAUCCGUCUGAGCU | 239 | AGCCAAGCUCAGACGGAU | 240 |
| | UGGCU | | CCGA | |
| miR-127-5p | CUGAAGCUCAGAGGGCU | 241 | AUCAGAGCCCUCUGAGCU | 242 |
| | CUGAU | | UCAG | |
| miR-1270 | CUGGAGAUAUGGAAGAG | 243 | ACACAGCUCUUCCAUAUC | 244 |
| | CUGUGU | | UCCAG | |
| miR-1271-3p | AGUGCCUGCUAUGUGCC | 245 | UGCCUGGCACAUAGCAGG | 246 |
| | AGGCA | | CACU | |
| miR-1271-5p | CUUGGCACCUAGCAAGC | 247 | UGAGUGCUUGCUAGGUGC | 248 |
| | ACUCA | | CAAG | |
| miR-1272 | GAUGAUGAUGGCAGCAA | 249 | UUUCAGAAUUUGCUGCCA | 250 |
| | AUUCUGAAA | | UCAUCAUC | |
| miR-1273a | GGGCGACAAAGCAAGAC | 251 | AAGAAAGAGUCUUGCUU | 252 |
| | UCUUUCUU | | UGUCGCCC | |
| miR-1273c | GGCGACAAAACGAGACC | 253 | GACAGGGUCUCGUUUUGU | 254 |
| | CUGUC | | CGCC | |
| miR-1273d | GAACCCAUGAGGUUGAG | 255 | ACUGCAGCCUCAACCUCA | 256 |
| | GCUGCAGU | | UGGGUUC | |
| miR-1273e | UUGCUUGAACCCAGGAA | 257 | UCCACUUCCUGGGUUCAA | 258 |
| | GUGGA | | GCAA | |
| miR-1273f | GGAGAUGGAGGUUGCAG | 259 | CACUGCAACCUCCAUCUC | 260 |
| | UG | | C | |
| miR-1273g-3p | ACCACUGCACUCCAGCC | 261 | CUCAGGCUGGAGUGCAGU | 262 |
| | UGAG | | GGU | |
| miR-1273g-5p | GGUGGUUGAGGCUGCAG | 263 | ACUUACUGCAGCCUCAAC | 264 |
| | UAAGU | | CACC | |
| miR-1275 | GUGGGGGAGAGGCUGUC | 265 | GACAGCCUCUCCCCCAC | 266 |
| miR-1276 | UAAAGAGCCCUGUGGAG | 267 | UGUCUCCACAGGGCUCUU | 268 |
| | ACA | | UA | |
| miR-1277-3p | UACGUAGAUAUAUAUGU | 269 | AAAAUACAUAUAUAUCU | 270 |
| | AUUUU | | ACGUA | |
| miR-1277-5p | AAAUAUAUAUAUAUAUG | 271 | AUACGUACAUAUAUAUA | 272 |
| | UACGUAU | | UAUAUUU | |
| miR-1278 | UAGUACUGUGCAUAUCA | 273 | AUAGAUGAUAUGCACAG | 274 |
| | UCUAU | | UACUA | |
| miR-1279 | UCAUAUUGCUUCUUUCU | 275 | AGAAAGAAGCAAUAUGA | 276 |
| miR-128 | UCACAGUGAACCGGUCU | 277 | AAAGAGACCGGUUCACUG | 278 |
| | CUUU | | UGA | |
| miR-1280 | UCCCACCGCUGCCACCC | 279 | GGGUGGCAGCGGUGGGA | 280 |
| miR-1281 | UCGCCUCCUCCUCUCCC | 281 | GGGAGAGGAGGAGGCGA | 282 |
| miR-1282 | UCGUUUGCCUUUUUCUG | 283 | AAGCAGAAAAAGGCAAAC | 284 |
| | CUU | | GA | |
| miR-1283 | UCUACAAAGGAAAGCGC | 285 | AGAAAGCGCUUUCCUUUG | 286 |
| | UUUCU | | UAGA | |
| miR-1284 | UCUAUACAGACCCUGGC | 287 | GAAAAGCCAGGGUCUGUA | 288 |
| | UUUUC | | UAGA | |
| miR-1285-3p | UCUGGGCAACAAAGUGA | 289 | AGGUCUCACUUUGUUGCC | 290 |
| | GACCU | | CAGA | |
| miR-1285-5p | GAUCUCACUUUGUUGCC | 291 | CCUGGGCAACAAAGUGAG | 292 |
| | CAGG | | AUC | |
| miR-1286 | UGCAGGACCAAGAUGAG | 293 | AGGGCUCAUCUUGGUCCU | 294 |
| | CCCU | | GCA | |
| miR-1287 | UGCUGGAUCAGUGGUUC | 295 | GACUCGAACCACUGAUCC | 296 |
| | GAGUC | | AGCA | |
| miR-1288 | UGGACUGCCCUGAUCUG | 297 | UCUCCAGAUCAGGGCAGU | 298 |
| | GAGA | | CCA | |
| miR-1289 | UGGAGUCCAGGAAUCUG | 299 | AAAAUGCAGAUUCCUGGA | 300 |
| | CAUUUU | | CUCCA | |
| miR-129-1-3p | AAGCCCUUACCCCAAAA | 301 | AUACUUUUUGGGGUAAG | 302 |
| | AGUAU | | GGCUU | |
| miR-129-2-3p | AAGCCCUUACCCCAAAA | 303 | AUGCUUUUUGGGGUAAG | 304 |
| | AGCAU | | GGCUU | |
| miR-129-5p | CUUUUUGCGGUCUGGGC | 305 | GCAAGCCCAGACCGCAAA | 306 |
| | UUGC | | AAG | |
| miR-1290 | UGGAUUUUUGGAUCAGG | 307 | UCCCUGAUCCAAAAAUCC | 308 |
| | GA | | A | |
| miR-1291 | UGGCCCUGACUGAAGAC | 309 | ACUGCUGGUCUUCAGUCA | 310 |
| | CAGCAGU | | GGGCCA | |
| miR-1292 | UGGGAACGGGUUCCGGC | 311 | CAGCGUCUGCCGGAACCC | 312 |
| | AGACGCUG | | GUUCCCA | |
| miR-1293 | UGGGUGGUCUGGAGAUU | 313 | GCACAAAUCUCCAGACCA | 314 |
| | UGUGC | | CCCA | |
| miR-1294 | UGUGAGGUUGGCAUUGU | 315 | AGACAACAAUGCCAACCU | 316 |
| | UGUCU | | CACA | |
| miR-1295a | UUAGGCCGCAGAUCUGG | 317 | UCACCCAGAUCUGCGGCC | 318 |
| | GUGA | | UAA | |
| miR-1295b-3p | AAUAGGCCACGGAUCUG | 319 | UUGCCCAGAUCCGUGGCC | 320 |
| | GGCAA | | UAUU | |
| miR-1295b-5p | CACCCAGAUCUGCGGCC | 321 | AUUAGGCCGCAGAUCUGG | 322 |
| | UAAU | | GUG | |
| miR-1296 | UUAGGGCCCUGGCUCCA | 323 | GGAGAUGGAGCCAGGGCC | 324 |
| | UCUCC | | CUAA | |
| miR-1297 | UUCAAGUAAUUCAGGUG | 325 | CACCUGAAUUACUUGAA | 326 |
| miR-1298 | UUCAUUCGGCUGUCCAG | 327 | UACAUCUGGACAGCCGAA | 328 |
| | AUGUA | | UGAA | |
| miR-1299 | UUCUGGAAUUCUGUGUG | 329 | UCCCUCACACAGAAUUCC | 330 |
| | AGGGA | | AGAA | |
| miR-1301 | UUGCAGCUGCCUGGGAG | 331 | GAAGUCACUCCCAGGCAG | 332 |
| | UGACUUC | | CUGCAA | |
| miR-1302 | UUGGGACAUACUUAUGC | 333 | UUUAGCAUAAGUAUGUCC | 334 |
| | UAAA | | CAA | |
| miR-1303 | UUUAGAGACGGGGUCUU | 335 | AGAGCAAGACCCCGUCUC | 336 |
| | GCUCU | | UAAA | |
| miR-1304-3p | UCUCACUGUAGCCUCGA | 337 | GGGGUUCGAGGCUACAGU | 338 |
| | ACCCC | | GAGA | |
| miR-1304-5p | UUUGAGGCUACAGUGAG | 339 | CACAUCUCACUGUAGCCU | 340 |
| | AUGUG | | CAAA | |
| miR-1305 | UUUUCAACUCUAAUGGG | 341 | UCUCUCCCAUUAGAGUUG | 342 |
| | AGAGA | | AAAA | |
| miR-1306-3p | ACGUUGGCUCUGGUGGU | 343 | CACCACCAGAGCCAACGU | 344 |
| | G | | | |
| miR-1306-5p | CCACCUCCCCUGCAAAC | 345 | UGGACGUUUGCAGGGGA | 346 |
| | GUCCA | | GGUGG | |
| miR-1307-3p | ACUCGGCGUGGCGUCGG | 347 | CACGACCGACGCCACGCC | 348 |
| | UCGUG | | GAGU | |
| miR-1307-5p | UCGACCGGACCUCGACC | 349 | AGCCGGUCGAGGUCCGGU | 350 |
| | GGCU | | CGA | |
| miR-130a-3p | CAGUGCAAUGUUAAAAG | 351 | AUGCCCUUUUAACAUUGC | 352 |
| | GGCAU | | ACUG | |
| miR-130a-5p | UUCACAUUGUGCUACUG | 353 | GCAGACAGUAGCACAAUG | 354 |
| | UCUGC | | UGAA | |
| miR-130b-3p | CAGUGCAAUGAUGAAAG | 355 | AUGCCCUUUCAUCAUUGC | 356 |
| | GGCAU | | ACUG | |
| miR-130b-5p | ACUCUUUCCCUGUUGCA | 357 | GUAGUGCAACAGGGAAA | 358 |
| | CUAC | | GAGU | |
| miR-132-3p | UAACAGUCUACAGCCAU | 359 | CGACCAUGGCUGUAGACU | 360 |
| | GGUCG | | GUUA | |
| miR-132-5p | ACCGUGGCUUUCGAUUG | 361 | AGUAACAAUCGAAAGCCA | 362 |
| | UUACU | | CGGU | |
| miR-1321 | CAGGGAGGUGAAUGUGA | 363 | AUCACAUUCACCUCCCUG | 364 |
| | U | | | |
| miR-1322 | GAUGAUGCUGCUGAUGC | 365 | CAGCAUCAGCAGCAUCAU | 366 |
| | UG | | C | |
| miR-1323 | UCAAAACUGAGGGGCAU | 367 | AGAAAAUGCCCCUCAGUU | 368 |
| | UUUCU | | UUGA | |
| miR-1324 | CCAGACAGAAUUCUAUG | 369 | GAAAGUGCAUAGAAUUC | 370 |
| | CACUUUC | | UGUCUGG | |
| miR-133a | UUUGGUCCCCUUCAACC | 371 | CAGCUGGUUGAAGGGGAC | 372 |
| | AGCUG | | CAAA | |
| miR-133b | UUUGGUCCCCUUCAACC | 373 | UAGCUGGUUGAAGGGGA | 374 |
| | AGCUA | | CCAAA | |
| miR-134 | UGUGACUGGUUGACCAG | 375 | CCCCUCUGGUCAACCAGU | 376 |
| | AGGGG | | CACA | |
| miR-1343 | CUCCUGGGGCCCGCACU | 377 | GCGAGAGUGCGGGCCCCA | 378 |
| | CUCGC | | GGAG | |
| miR-135a-3p | UAUAGGGAUUGGAGCCG | 379 | CGCCACGGCUCCAAUCCC | 380 |
| | UGGCG | | UAUA | |
| miR-135a-5p | UAUGGCUUUUUAUUCCU | 381 | UCACAUAGGAAUAAAAA | 382 |
| | AUGUGA | | GCCAUA | |
| miR-135b-3p | AUGUAGGGCUAAAAGCC | 383 | CCCAUGGCUUUUAGCCCU | 384 |
| | AUGGG | | ACAU | |
| miR-135b-5p | UAUGGCUUUUCAUUCCU | 385 | UCACAUAGGAAUGAAAA | 386 |
| | AUGUGA | | GCCAUA | |
| miR-136-3p | CAUCAUCGUCUCAAAUG | 387 | AGACUCAUUUGAGACGAU | 388 |
| | AGUCU | | GAUG | |
| miR-136-5p | ACUCCAUUUGUUUUGAU | 389 | UCCAUCAUCAAAACAAAU | 390 |
| | GAUGGA | | GGAGU | |
| miR-137 | UUAUUGCUUAAGAAUAC | 391 | CUACGCGUAUUCUUAAGC | 392 |
| | GCGUAG | | AAUAA | |
| miR-138-1-3p | GCUACUUCACAACACCA | 393 | GGCCCUGGUGUUGUGAAG | 394 |
| | GGGCC | | UAGC | |
| miR-138-2-3p | GCUAUUUCACGACACCA | 395 | AACCCUGGUGUCGUGAAA | 396 |
| | GGGUU | | UAGC | |
| miR-138-5p | AGCUGGUGUUGUGAAUC | 397 | CGGCCUGAUUCACAACAC | 398 |
| | AGGCCG | | CAGCU | |
| miR-139-3p | GGAGACGCGGCCCUGUU | 399 | ACUCCAACAGGGCCGCGU | 400 |
| | GGAGU | | CUCC | |
| miR-139-5p | UCUACAGUGCACGUGUC | 401 | CUGGAGACACGUGCACUG | 402 |
| | UCCAG | | UAGA | |
| miR-140-3p | UACCACAGGGUAGAACC | 403 | CCGUGGUUCUACCCUGUG | 404 |
| | ACGG | | GUA | |
| miR-140-5p | CAGUGGUUUUACCCUAU | 405 | CUACCAUAGGGUAAAACC | 406 |
| | GGUAG | | ACUG | |
| miR-141-3p | UAACACUGUCUGGUAAA | 407 | CCAUCUUUACCAGACAGU | 408 |
| | GAUGG | | GUUA | |
| miR-141-5p | CAUCUUCCAGUACAGUG | 409 | UCCAACACUGUACUGGAA | 410 |
| | UUGGA | | GAUG | |
| miR-142-3p | UGUAGUGUUUCCUACUU | 411 | UCCAUAAAGUAGGAAACA | 412 |
| | UAUGGA | | CUACA | |
| miR-142-5p | CAUAAAGUAGAAAGCAC | 413 | AGUAGUGCUUUCUACUUU | 414 |
| | UACU | | AUG | |
| miR-143-3p | UGAGAUGAAGCACUGUA | 415 | GAGCUACAGUGCUUCAUC | 416 |
| | GCUC | | UCA | |
| miR-143-5p | GGUGCAGUGCUGCAUCU | 417 | ACCAGAGAUGCAGCACUG | 418 |
| | CUGGU | | CACC | |
| miR-144-3p | UACAGUAUAGAUGAUGU | 419 | AGUACAUCAUCUAUACUG | 420 |
| | ACU | | UA | |
| miR-144-5p | GGAUAUCAUCAUAUACU | 421 | CUUACAGUAUAUGAUGA | 422 |
| | GUAAG | | UAUCC | |
| miR-145-3p | GGAUUCCUGGAAAUACU | 423 | AGAACAGUAUUUCCAGGA | 424 |
| | GUUCU | | AUCC | |
| miR-145-5p | GUCCAGUUUUCCCAGGA | 425 | AGGGAUUCCUGGGAAAAC | 426 |
| | AUCCCU | | UGGAC | |
| miR-1468 | CUCCGUUUGCCUGUUUC | 427 | CAGCGAAACAGGCAAACG | 428 |
| | GCUG | | GAG | |
| miR-1469 | CUCGGCGCGGGGCGCGG | 429 | GGAGCCCGCGCCCCGCGC | 430 |
| | GCUCC | | CGAG | |
| miR-146a-3p | CCUCUGAAAUUCAGUUC | 431 | CUGAAGAACUGAAUUUCA | 432 |
| | UUCAG | | GAGG | |
| miR-146a-5p | UGAGAACUGAAUUCCAU | 433 | AACCCAUGGAAUUCAGUU | 434 |
| | GGGUU | | CUCA | |
| miR-146b-3p | UGCCCUGUGGACUCAGU | 435 | CCAGAACUGAGUCCACAG | 436 |
| | UCUGG | | GGCA | |
| miR-146b-5p | UGAGAACUGAAUUCCAU | 437 | AGCCUAUGGAAUUCAGUU | 438 |
| | AGGCU | | CUCA | |
| miR-1470 | GCCCUCCGCCCGUGCACC | 439 | CGGGGUGCACGGGCGGAG | 440 |
| | CCG | | GGC | |
| miR-1471 | GCCCGCGUGUGGAGCCA | 441 | ACACCUGGCUCCACACGC | 442 |
| | GGUGU | | GGGC | |
| miR-147a | GUGUGUGGAAAUGCUUC | 443 | GCAGAAGCAUUUCCACAC | 444 |
| | UGC | | AC | |
| miR-147b | GUGUGCGGAAAUGCUUC | 445 | UAGCAGAAGCAUUUCCGC | 446 |
| | UGCUA | | ACAC | |
| miR-148a-3p | UCAGUGCACUACAGAAC | 447 | ACAAAGUUCUGUAGUGCA | 448 |
| | UUUGU | | CUGA | |
| miR-148a-5p | AAAGUUCUGAGACACUC | 449 | AGUCGGAGUGUCUCAGAA | 450 |
| | CGUU | | CUUU | |
| miR-148b-3p | UCAGUGCAUCACAGAAC | 451 | ACAAAGUUCUGUGAUGCA | 452 |
| | UUUGU | | CUGA | |
| miR-148b-5p | AAGUUCUGUUAUACACU | 453 | GCCUGAGUGUAUAACAGA | 454 |
| | CAGGC | | ACUU | |
| miR-149-3p | AGGGAGGGACGGGGGCU | 455 | GCACAGCCCCCGUCCCUC | 456 |
| | GUGC | | CCU | |
| miR-149-5p | UCUGGCUCCGUGUCUUC | 457 | GGGAGUGAAGACACGGA | 458 |
| | ACUCCC | | GCCAGA | |
| miR-150-3p | CUGGUACAGGCCUGGGG | 459 | CUGUCCCCCAGGCCUGUA | 460 |
| | GACAG | | CCAG | |
| miR-150-5p | UCUCCCAACCCUUGUAC | 461 | CACUGGUACAAGGGUUGG | 462 |
| | CAGUG | | GAGA | |
| miR-151a-3p | CUAGACUGAAGCUCCUU | 463 | CCUCAAGGAGCUUCAGUC | 464 |
| | GAGG | | UAG | |
| miR-151a-5p | UCGAGGAGCUCACAGUC | 465 | ACUAGACUGUGAGCUCCU | 466 |
| | UAGU | | CGA | |
| miR-151b | UCGAGGAGCUCACAGUC | 467 | AGACUGUGAGCUCCUCGA | 468 |
| | U | | | |
| miR-152 | UCAGUGCAUGACAGAAC | 469 | CCAAGUUCUGUCAUGCAC | 470 |
| | UUGG | | UGA | |
| miR-153 | UUGCAUAGUCACAAAAG | 471 | GAUCACUUUUGUGACUAU | 472 |
| | UGAUC | | GCAA | |
| miR-1537 | AAAACCGUCUAGUUACA | 473 | ACAACUGUAACUAGACGG | 474 |
| | GUUGU | | UUUU | |
| miR-1538 | CGGCCCGGGCUGCUGCU | 475 | AGGAACAGCAGCAGCCCG | 476 |
| | GUUCCU | | GGCCG | |
| miR-1539 | UCCUGCGCGUCCCAGAU | 477 | GGGCAUCUGGGACGCGCA | 478 |
| | GCCC | | GGA | |
| miR-154-3p | AAUCAUACACGGUUGAC | 479 | AAUAGGUCAACCGUGUAU | 480 |
| | CUAUU | | GAUU | |
| miR-154-5p | UAGGUUAUCCGUGUUGC | 481 | CGAAGGCAACACGGAUAA | 482 |
| | CUUCG | | CCUA | |
| miR-155-3p | CUCCUACAUAUUAGCAU | 483 | UGUUAAUGCUAAUAUGU | 484 |
| | UAACA | | AGGAG | |
| miR-155-5p | UUAAUGCUAAUCGUGAU | 485 | ACCCCUAUCACGAUUAGC | 486 |
| | AGGGGU | | AUUAA | |
| miR-1587 | UUGGGCUGGGCUGGGUU | 487 | CCCAACCCAGCCCAGCCC | 488 |
| | GGG | | AA | |
| miR-15a-3p | CAGGCCAUAUUGUGCUG | 489 | UGAGGCAGCACAAUAUGG | 490 |
| | CCUCA | | CCUG | |
| miR-15a-5p | UAGCAGCACAUAAUGGU | 491 | CACAAACCAUUAUGUGCU | 492 |
| | UUGUG | | GCUA | |
| miR-15b-3p | CGAAUCAUUAUUUGCUG | 493 | UAGAGCAGCAAAUAAUG | 494 |
| | CUCUA | | AUUCG | |
| miR-15b-5p | UAGCAGCACAUCAUGGU | 495 | UGUAAACCAUGAUGUGCU | 496 |
| | UUACA | | GCUA | |
| miR-16-1-3p | CCAGUAUUAACUGUGCU | 497 | UCAGCAGCACAGUUAAUA | 498 |
| | GCUGA | | CUGG | |
| miR-16-2-3p | CCAAUAUUACUGUGCUG | 499 | UAAAGCAGCACAGUAAUA | 500 |
| | CUUUA | | UUGG | |
| miR-16-5p | UAGCAGCACGUAAAUAU | 501 | CGCCAAUAUUUACGUGCU | 502 |
| | UGGCG | | GCUA | |
| miR-17-3p | ACUGCAGUGAAGGCACU | 503 | CUACAAGUGCCUUCACUG | 504 |
| | UGUAG | | CAGU | |
| miR-17-5p | CAAAGUGCUUACAGUGC | 505 | CUACCUGCACUGUAAGCA | 506 |
| | AGGUAG | | CUUUG | |
| miR-181a-2-3p | ACCACUGACCGUUGACU | 507 | GGUACAGUCAACGGUCAG | 508 |
| | GUACC | | UGGU | |
| miR-181a-3p | ACCAUCGACCGUUGAUU | 509 | GGUACAAUCAACGGUCGA | 510 |
| | GUACC | | UGGU | |
| miR-181a-5p | AACAUUCAACGCUGUCG | 511 | ACUCACCGACAGCGUUGA | 512 |
| | GUGAGU | | AUGUU | |
| miR-181b-3p | CUCACUGAACAAUGAAU | 513 | UUGCAUUCAUUGUUCAGU | 514 |
| | GCAA | | GAG | |
| miR-181b-5p | AACAUUCAUUGCUGUCG | 515 | ACCCACCGACAGCAAUGA | 516 |
| | GUGGGU | | AUGUU | |
| miR-181c-3p | AACCAUCGACCGUUGAG | 517 | GUCCACUCAACGGUCGAU | 518 |
| | UGGAC | | GGUU | |
| miR-181c-5p | AACAUUCAACCUGUCGG | 519 | ACUCACCGACAGGUUGAA | 520 |
| | UGAGU | | UGUU | |
| miR-181d | AACAUUCAUUGUUGUCG | 521 | ACCCACCGACAACAAUGA | 522 |
| | GUGGGU | | AUGUU | |
| miR-182-3p | UGGUUCUAGACUUGCCA | 523 | UAGUUGGCAAGUCUAGA | 524 |
| | ACUA | | ACCA | |
| miR-182-5p | UUUGGCAAUGGUAGAAC | 525 | AGUGUGAGUUCUACCAUU | 526 |
| | UCACACU | | GCCAAA | |
| miR-1825 | UCCAGUGCCCUCCUCUC | 527 | GGAGAGGAGGGCACUGG | 528 |
| | C | | A | |
| miR-1827 | UGAGGCAGUAGAUUGAA | 529 | AUUCAAUCUACUGCCUCA | 530 |
| | U | | | |
| miR-183-3p | GUGAAUUACCGAAGGGC | 531 | UUAUGGCCCUUCGGUAAU | 532 |
| | CAUAA | | UCAC | |
| miR-183-5p | UAUGGCACUGGUAGAAU | 533 | AGUGAAUUCUACCAGUGC | 534 |
| | UCACU | | CAUA | |
| miR-184 | UGGACGGAGAACUGAUA | 535 | ACCCUUAUCAGUUCUCCG | 536 |
| | AGGGU | | UCCA | |
| miR-185-3p | AGGGGCUGGCUUUCCUC | 537 | GACCAGAGGAAAGCCAGC | 538 |
| | UGGUC | | CCCU | |
| miR-185-5p | UGGAGAGAAAGGCAGUU | 539 | UCAGGAACUGCCUUUCUC | 540 |
| | CCUGA | | UCCA | |
| miR-186-3p | GCCCAAAGGUGAAUUUU | 541 | CCCAAAAAAUUCACCUUU | 542 |
| | UUGGG | | GGGC | |
| miR-186-5p | CAAAGAAUUCUCCUUUU | 543 | AGCCCAAAAGGAGAAUUC | 544 |
| | GGGCU | | UUUG | |
| miR-187-3p | UCGUGUCUUGUGUUGCA | 545 | CCGGCUGCAACACAAGAC | 546 |
| | GCCGG | | ACGA | |
| miR-187-5p | GGCUACAACACAGGACC | 547 | GCCCGGGUCCUGUGUUGU | 548 |
| | CGGGC | | AGCC | |
| miR-188-3p | CUCCCACAUGCAGGGUU | 549 | UGCAAACCCUGCAUGUGG | 550 |
| | UGCA | | GAG | |
| miR-188-5p | CAUCCCUUGCAUGGUGG | 551 | CCCUCCACCAUGCAAGGG | 552 |
| | AGGG | | AUG | |
| miR-18a-3p | ACUGCCCUAAGUGCUCC | 553 | CCAGAAGGAGCACUUAGG | 554 |
| | UUCUGG | | GCAGU | |
| miR-18a-5p | UAAGGUGCAUCUAGUGC | 555 | CUAUCUGCACUAGAUGCA | 556 |
| | AGAUAG | | CCUUA | |
| miR-18b-3p | UGCCCUAAAUGCCCCUU | 557 | GCCAGAAGGGGCAUUUAG | 558 |
| | CUGGC | | GGCA | |
| miR-18b-5p | UAAGGUGCAUCUAGUGC | 559 | CUAACUGCACUAGAUGCA | 560 |
| | AGUUAG | | CCUUA | |
| miR-1908 | CGGCGGGGACGGCGAUU | 561 | GACCAAUCGCCGUCCCCG | 562 |
| | GGUC | | CCG | |
| miR-1909-3p | CGCAGGGGCCGGGUGCU | 563 | CGGUGAGCACCCGGCCCC | 564 |
| | CACCG | | UGCG | |
| miR-1909-5p | UGAGUGCCGGUGCCUGC | 565 | CAGGGCAGGCACCGGCAC | 566 |
| | CCUG | | UCA | |
| miR-190a | UGAUAUGUUUGAUAUAU | 567 | ACCUAAUAUAUCAAACAU | 568 |
| | UAGGU | | AUCA | |
| miR-190b | UGAUAUGUUUGAUAUUG | 569 | AACCCAAUAUCAAACAUA | 570 |
| | GGUU | | UCA | |
| miR-191-3p | GCUGCGCUUGGAUUUCG | 571 | GGGGACGAAAUCCAAGCG | 572 |
| | UCCCC | | CAGC | |
| miR-191-5p | CAACGGAAUCCCAAAAG | 573 | CAGCUGCUUUUGGGAUUC | 574 |
| | CAGCUG | | CGUUG | |
| miR-1910 | CCAGUCCUGUGCCUGCC | 575 | AGGCGGCAGGCACAGGAC | 576 |
| | GCCU | | UGG | |
| miR-1911-3p | CACCAGGCAUUGUGGUC | 577 | GGAGACCACAAUGCCUGG | 578 |
| | UCC | | UG | |
| miR-1911-5p | UGAGUACCGCCAUGUCU | 579 | CCCAACAGACAUGGCGGU | 580 |
| | GUUGGG | | ACUCA | |
| miR-1912 | UACCCAGAGCAUGCAGU | 581 | UUCACACUGCAUGCUCUG | 582 |
| | GUGAA | | GGUA | |
| miR-1913 | UCUGCCCCCUCCGCUGC | 583 | UGGCAGCAGCGGAGGGGG | 584 |
| | UGCCA | | CAGA | |
| miR-1914-3p | GGAGGGGUCCCGCACUG | 585 | CCUCCCAGUGCGGGACCC | 586 |
| | GGAGG | | CUCC | |
| miR-1914-5p | CCCUGUGCCCGGCCCAC | 587 | CAGAAGUGGGCCGGGCAC | 588 |
| | UUCUG | | AGGG | |
| miR-1915-3p | CCCCAGGGCGACGCGGC | 589 | CCCGCCGCGTCGCCCTGG | 590 |
| | GGG | | GG | |
| miR-1915-5p | ACCUUGCCUUGCUGCCC | 591 | GGCCCGGGCAGCAAGGCA | 592 |
| | GGGCC | | AGGU | |
| miR-192-3p | CUGCCAAUUCCAUAGGU | 593 | CUGUGACCUAUGGAAUUG | 594 |
| | CACAG | | GCAG | |
| miR-192-5p | CUGACCUAUGAAUUGAC | 595 | GGCUGUCAAUUCAUAGGU | 596 |
| | AGCC | | CAG | |
| miR-193a-3p | AACUGGCCUACAAAGUC | 597 | ACUGGGACUUUGUAGGCC | 598 |
| | CCAGU | | AGUU | |
| miR-193a-5p | UGGGUCUUUGCGGGCGA | 599 | UCAUCUCGCCCGCAAAGA | 600 |
| | GAUGA | | CCCA | |
| miR-193b-3p | AACUGGCCCUCAAAGUC | 601 | AGCGGGACUUUGAGGGCC | 602 |
| | CCGCU | | AGUU | |
| miR-193b-5p | CGGGGUUUUGAGGGCGA | 603 | UCAUCUCGCCCUCAAAAC | 604 |
| | GAUGA | | CCCG | |
| miR-194-3p | CCAGUGGGGCUGCUGUU | 605 | CAGAUAACAGCAGCCCCA | 606 |
| | AUCUG | | CUGG | |
| miR-194-5p | UGUAACAGCAACUCCAU | 607 | UCCACAUGGAGUUGCUGU | 608 |
| | GUGGA | | UACA | |
| miR-195-3p | CCAAUAUUGGCUGUGCU | 609 | GGAGCAGCACAGCCAAUA | 610 |
| | GCUCC | | UUGG | |
| miR-195-5p | UAGCAGCACAGAAAUAU | 611 | GCCAAUAUUUCUGUGCUG | 612 |
| | UGGC | | CUA | |
| miR-196a-3p | CGGCAACAAGAAACUGC | 613 | CUCAGGCAGUUUCUUGUU | 614 |
| | CUGAG | | GCCG | |
| miR-196a-5p | UAGGUAGUUUCAUGUUG | 615 | CCCAACAACAUGAAACUA | 616 |
| | UUGGG | | CCUA | |
| miR-196b-3p | UCGACAGCACGACACUG | 617 | GAAGGCAGUGUCGUGCUG | 618 |
| | CCUUC | | UCGA | |
| miR-196b-5p | UAGGUAGUUUCCUGUUG | 619 | CCCAACAACAGGAAACUA | 620 |
| | UUGGG | | CCUA | |
| miR-197-3p | UUCACCACCUUCUCCAC | 621 | GCUGGGUGGAGAAGGUG | 622 |
| | CCAGC | | GUGAA | |
| miR-197-5p | CGGGUAGAGAGGGCAGU | 623 | CCUCCCACUGCCCUCUCU | 624 |
| | GGGAGG | | ACCCG | |
| miR-1972 | UCAGGCCAGGCACAGUG | 625 | UGAGCCACUGUGCCUGGC | 626 |
| | GCUCA | | CUGA | |
| miR-1973 | ACCGUGCAAAGGUAGCAA | 627 | UAUGCUACCUUUGCACGG | 628 |
| | U | | U | |
| miR-1976 | CCUCCUGCCCUCCUUGC | 629 | ACAGCAAGGAGGGCAGGA | 630 |
| | UGU | | GG | |
| miR-198 | GGUCCAGAGGGGAGAUA | 631 | GAACCUAUCUCCCCUCUG | 632 |
| | GGUUC | | GACC | |
| miR-199a-3p | ACAGUAGUCUGCACAUU | 633 | UAACCAAUGUGCAGACUA | 634 |
| | GGUUA | | CUGU | |
| miR-199a-5p | CCCAGUGUUCAGACUAC | 635 | GAACAGGUAGUCUGAACA | 636 |
| | CUGUUC | | CUGGG | |
| miR-199b-3p | ACAGUAGUCUGCACAUU | 637 | UAACCAAUGUGCAGACUA | 638 |
| | GGUUA | | CUGU | |
| miR-199b-5p | CCCAGUGUUUAGACUAU | 639 | GAACAGAUAGUCUAAACA | 640 |
| | CUGUUC | | CUGGG | |
| miR-19a-3p | UGUGCAAAUCUAUGCAA | 641 | UCAGUUUUGCAUAGAUU | 642 |
| | AACUGA | | UGCACA | |
| miR-19a-5p | AGUUUUGCAUAGUUGCA | 643 | UGUAGUGCAACUAUGCAA | 644 |
| | CUACA | | AACU | |
| miR-19b-1-5p | AGUUUUGCAGGUUUGCA | 645 | GCUGGAUGCAAACCUGCA | 646 |
| | UCCAGC | | AAACU | |
| miR-19b-2-5p | AGUUUUGCAGGUUUGCA | 647 | UGAAAUGCAAACCUGCAA | 648 |
| | UUUCA | | AACU | |
| miR-19b-3p | UGUGCAAAUCCAUGCAA | 649 | UCAGUUUUGCAUGGAUU | 650 |
| | AACUGA | | UGCACA | |
| miR-200a-3p | UAACACUGUCUGGUAAC | 651 | ACAUCGUUACCAGACAGU | 652 |
| | GAUGU | | GUUA | |
| miR-200a-5p | CAUCUUACCGGACAGUG | 653 | UCCAGCACUGUCCGGUAA | 654 |
| | CUGGA | | GAUG | |
| miR-200b-3p | UAAUACUGCCUGGUAAU | 655 | UCAUCAUUACCAGGCAGU | 656 |
| | GAUGA | | AUUA | |
| miR-200b-5p | CAUCUUACUGGGCAGCA | 657 | UCCAAUGCUGCCCAGUAA | 658 |
| | UUGGA | | GAUG | |
| miR-200c-3p | UAAUACUGCCGGGUAAU | 659 | UCCAUCAUUACCCGGCAG | 660 |
| | GAUGGA | | UAUUA | |
| miR-200c-5p | CGUCUUACCCAGCAGUG | 661 | CCAAACACUGCUGGGUAA | 662 |
| | UUUGG | | GACG | |
| miR-202-3p | AGAGGUAUAGGGCAUGG | 663 | UUCCCAUGCCCUAUACCU | 664 |
| | GAA | | CU | |
| miR-202-5p | UUCCUAUGCAUAUACUU | 665 | CAAAGAAGUAUAUGCAU | 666 |
| | CUUUG | | AGGAA | |
| miR-203 | GUGAAAUGUUUAGGACC | 667 | CUAGUGGUCCUAAACAUU | 668 |
| | ACUAG | | UCAC | |
| miR-204-3p | GCUGGGAAGGCAAAGGG | 669 | ACGUCCCUUUGCCUUCCC | 670 |
| | ACGU | | AGC | |
| miR-204-5p | UUCCCUUUGUCAUCCUA | 671 | AGGCAUAGGAUGACAAA | 672 |
| | UGCCU | | GGGAA | |
| miR-205-3p | GAUUUCAGUGGAGUGAA | 673 | GAACUUCACUCCACUGAA | 674 |
| | GUUC | | AUC | |
| miR-205-5p | UCCUUCAUUCCACCGGA | 675 | CAGACUCCGGUGGAAUGA | 676 |
| | GUCUG | | AGGA | |
| miR-2052 | UGUUUUGAUAACAGUAA | 677 | ACAUUACUGUUAUCAAAA | 678 |
| | UGU | | CA | |
| miR-2053 | GUGUUAAUUAAACCUCU | 679 | GUAAAUAGAGGUUUAAU | 680 |
| | AUUUAC | | UAACAC | |
| miR-2054 | CUGUAAUAUAAAUUUAA | 681 | AAUAAAUUAAAUUUAUA | 682 |
| | UUUAUU | | UUACAG | |
| miR-206 | UGGAAUGUAAGGAAGUG | 683 | CCACACACUUCCUUACAU | 684 |
| | UGUGG | | UCCA | |
| miR-208a | AUAAGACGAGCAAAAAG | 685 | ACAAGCUUUUUGCUCGUC | 686 |
| | CUUGU | | UUAU | |
| miR-208b | AUAAGACGAACAAAAGG | 687 | ACAAACCUUUUGUUCGUC | 688 |
| | UUUGU | | UUAU | |
| miR-20a-3p | ACUGCAUUAUGAGCACU | 689 | CUUUAAGUGCUCAUAAUG | 690 |
| | UAAAG | | CAGU | |
| miR-20a-5p | UAAAGUGCUUAUAGUGC | 691 | CUACCUGCACUAUAAGCA | 692 |
| | AGGUAG | | CUUUA | |
| miR-20b-3p | ACUGUAGUAUGGGCACU | 693 | CUGGAAGUGCCCAUACUA | 694 |
| | UCCAG | | CAGU | |
| miR-20b-5p | CAAAGUGCUCAUAGUGC | 695 | CUACCUGCACUAUGAGCA | 696 |
| | AGGUAG | | CUUUG | |
| miR-21-3p | CAACACCAGUCGAUGGG | 697 | ACAGCCCAUCGACUGGUG | 698 |
| | CUGU | | UUG | |
| miR-21-5p | UAGCUUAUCAGACUGAU | 699 | UCAACAUCAGUCUGAUAA | 700 |
| | GUUGA | | GCUA | |
| miR-210 | CUGUGCGUGUGACAGCG | 701 | UCAGCCGCUGUCACACGC | 702 |
| | GCUGA | | ACAG | |
| miR-211-3p | GCAGGGACAGCAAAGGG | 703 | GCACCCCUUUGCUGUCCC | 704 |
| | GUGC | | UGC | |
| miR-211-5p | UUCCCUUUGUCAUCCUU | 705 | AGGCGAAGGAUGACAAA | 706 |
| | CGCCU | | GGGAA | |
| miR-2110 | UUGGGGAAACGGCCGCU | 707 | CACUCAGCGGCCGUUUCC | 708 |
| | GAGUG | | CCAA | |
| miR-2113 | AUUUGUGCUUGGCUCUG | 709 | GUGACAGAGCCAAGCACA | 710 |
| | UCAC | | AAU | |
| miR-2114-3p | CGAGCCUCAAGCAAGGG | 711 | AAGUCCCUUGCUUGAGGC | 712 |
| | ACUU | | UCG | |
| miR-2114-5p | UAGUCCCUUCCUUGAAG | 713 | GACCGCUUCAAGGAAGGG | 714 |
| | CGGUC | | ACUA | |
| miR-2115-3p | CAUCAGAAUUCAUGGAG | 715 | CUAGCCUCCAUGAAUUCU | 716 |
| | GCUAG | | GAUG | |
| miR-2115-5p | AGCUUCCAUGACUCCUG | 717 | UCCAUCAGGAGUCAUGGA | 718 |
| | AUGGA | | AGCU | |
| miR-2116-3p | CCUCCCAUGCCAAGAAC | 719 | GGGAGUUCUUGGCAUGG | 720 |
| | UCCC | | GAGG | |
| miR-2116-5p | GGUUCUUAGCAUAGGAG | 721 | AGACCUCCUAUGCUAAGA | 722 |
| | GUCU | | ACC | |
| miR-2117 | UGUUCUCUUUGCCAAGG | 723 | CUGUCCUUGGCAAAGAGA | 724 |
| | ACAG | | ACA | |
| miR-212-3p | UAACAGUCUCCAGUCAC | 725 | GGCCGUGACUGGAGACUG | 726 |
| | GGCC | | UUA | |
| miR-212-5p | ACCUUGGCUCUAGACUG | 727 | AGUAAGCAGUCUAGAGCC | 728 |
| | CUUACU | | AAGGU | |
| miR-214-3p | ACAGCAGGCACAGACAG | 729 | ACUGCCUGUCUGUGCCUG | 730 |
| | GCAGU | | CUGU | |
| miR-214-5p | UGCCUGUCUACACUUGC | 731 | GCACAGCAAGUGUAGACA | 732 |
| | UGUGC | | GGCA | |
| miR-215 | AUGACCUAUGAAUUGAC | 733 | GUCUGUCAAUUCAUAGGU | 734 |
| | AGAC | | CAU | |
| miR-216a | UAAUCUCAGCUGGCAAC | 735 | UCACAGUUGCCAGCUGAG | 736 |
| | UGUGA | | AUUA | |
| miR-216b | AAAUCUCUGCAGGCAAA | 737 | UCACAUUUGCCUGCAGAG | 738 |
| | UGUGA | | AUUU | |
| miR-217 | UACUGCAUCAGGAACUG | 739 | UCCAAUCAGUUCCUGAUG | 740 |
| | AUUGGA | | CAGUA | |
| miR-218-1-3p | AUGGUUCCGUCAAGCAC | 741 | CCAUGGUGCUUGACGGAA | 742 |
| | CAUGG | | CCAU | |
| miR-218-2-3p | CAUGGUUCUGUCAAGCA | 743 | CGCGGUGCUUGACAGAAC | 744 |
| | CCGCG | | CAUG | |
| miR-218-5p | UUGUGCUUGAUCUAACC | 745 | ACAUGGUUAGAUCAAGCA | 746 |
| | AUGU | | CAA | |
| miR-219-1-3p | AGAGUUGAGUCUGGACG | 747 | CGGGACGUCCAGACUCAA | 748 |
| | UCCCG | | CUCU | |
| miR-219-2-3p | AGAAUUGUGGCUGGACA | 749 | ACAGAUGUCCAGCCACAA | 750 |
| | UCUGU | | UUCU | |
| miR-219-5p | UGAUUGUCCAAACGCAA | 751 | AGAAUUGCGUUUGGACA | 752 |
| | UUCU | | AUCA | |
| miR-22-3p | AAGCUGCCAGUUGAAGA | 753 | ACAGUUCUUCAACUGGCA | 754 |
| | ACUGU | | GCUU | |
| miR-22-5p | AGUUCUUCAGUGGCAAG | 755 | UAAAGCUUGCCACUGAAG | 756 |
| | CUUUA | | AACU | |
| miR-221-3p | AGCUACAUUGUCUGCUG | 757 | GAAACCCAGCAGACAAUG | 758 |
| | GGUUUC | | UAGCU | |
| miR-221-5p | ACCUGGCAUACAAUGUA | 759 | AAAUCUACAUUGUAUGCC | 760 |
| | GAUUU | | AGGU | |
| miR-222-3p | AGCUACAUCUGGCUACU | 761 | ACCCAGUAGCCAGAUGUA | 762 |
| | GGGU | | GCU | |
| miR-222-5p | CUCAGUAGCCAGUGUAG | 763 | AGGAUCUACACUGGCUAC | 764 |
| | AUCCU | | UGAG | |
| miR-223-3p | UGUCAGUUUGUCAAAUA | 765 | UGGGGUAUUUGACAAAC | 766 |
| | CCCCA | | UGACA | |
| miR-223-5p | CGUGUAUUUGACAAGCU | 767 | AACUCAGCUUGUCAAAUA | 768 |
| | GAGUU | | CACG | |
| miR-224-3p | AAAAUGGUGCCCUAGUG | 769 | UGUAGUCACUAGGGCACC | 770 |
| | ACUACA | | AUUUU | |
| miR-224-5p | CAAGUCACUAGUGGUUC | 771 | AACGGAACCACUAGUGAC | 772 |
| | CGUU | | UUG | |
| miR-2276 | UCUGCAAGUGUCAGAGG | 773 | CCUCGCCUCUGACACUUG | 774 |
| | CGAGG | | CAGA | |
| miR-2277-3p | UGACAGCGCCCUGCCUG | 775 | GAGCCAGGCAGGGCGCUG | 776 |
| | GCUC | | UCA | |
| miR-2277-5p | AGCGCGGGCUGAGCGCU | 777 | GACUGGCAGCGCUCAGCC | 778 |
| | GCCAGUC | | CGCGCU | |
| miR-2278 | GAGAGCAGUGUGUGUUG | 779 | CCAGGCAACACACACUGC | 780 |
| | CCUGG | | UCUC | |
| miR-2355-3p | AUUGUCCUUGCUGUUUG | 781 | AUCUCCAAACAGCAAGGA | 782 |
| | GAGAU | | CAAU | |
| miR-2355-5p | AUCCCCAGAUACAAUGG | 783 | UUGUCCAUUGUAUCUGGG | 784 |
| | ACAA | | GAU | |
| miR-2392 | UAGGAUGGGGGUGAGAG | 785 | CACCUCUCACCCCCAUCC | 786 |
| | GUG | | UA | |
| miR-23a-3p | AUCACAUUGCCAGGGAU | 787 | GGAAAUCCCUGGCAAUGU | 788 |
| | UUCC | | GAU | |
| miR-23a-5p | GGGGUUCCUGGGGAUGG | 789 | AAAUCCCAUCCCCAGGAA | 790 |
| | GAUUU | | CCCC | |
| miR-23b-3p | AUCACAUUGCCAGGGAU | 791 | GGUAAUCCCUGGCAAUGU | 792 |
| | UACC | | GAU | |
| miR-23b-5p | UGGGUUCCUGGCAUGCU | 793 | AAAUCAGCAUGCCAGGAA | 794 |
| | GAUUU | | CCCA | |
| miR-23c | AUCACAUUGCCAGUGAU | 795 | GGGUAAUCACUGGCAAUG | 796 |
| | UACCC | | UGAU | |
| miR-24-1-5p | UGCCUACUGAGCUGAUA | 797 | ACUGAUAUCAGCUCAGUA | 798 |
| | UCAGU | | GGCA | |
| miR-24-2-5p | UGCCUACUGAGCUGAAA | 799 | CUGUGUUUCAGCUCAGUA | 800 |
| | CACAG | | GGCA | |
| miR-24-3p | UGGCUCAGUUCAGCAGG | 801 | CUGUUCCUGCUGAACUGA | 802 |
| | AACAG | | GCCA | |
| miR-2467-3p | AGCAGAGGCAGAGAGGC | 803 | CCUGAGCCUCUCUGCCUC | 804 |
| | UCAGG | | UGCU | |
| miR-2467-5p | UGAGGCUCUGUUAGCCU | 805 | GAGCCAAGGCUAACAGAG | 806 |
| | UGGCUC | | CCUCA | |
| miR-25-3p | CAUUGCACUUGUCUCGG | 807 | UCAGACCGAGACAAGUGC | 808 |
| | UCUGA | | AAUG | |
| miR-25-5p | AGGCGGAGACUUGGGCA | 809 | CAAUUGCCCAAGUCUCCG | 810 |
| | AUUG | | CCU | |
| miR-2681-3p | UAUCAUGGAGUUGGUAA | 811 | GUGCUUUACCAACUCCAU | 812 |
| | AGCAC | | GAUA | |
| miR-2681-5p | GUUUUACCACCUCCAGG | 813 | AGUCUCCUGGAGGUGGUA | 814 |
| | AGACU | | AAAC | |
| miR-2682-3p | CGCCUCUUCAGCGCUGU | 815 | GGAAGACAGCGCUGAAGA | 816 |
| | CUUCC | | GGCG | |
| miR-2682-5p | CAGGCAGUGACUGUUCA | 817 | GACGUCUGAACAGUCACU | 818 |
| | GACGUC | | GCCUG | |
| miR-26a-1-3p | CCUAUUCUUGGUUACUU | 819 | CGUGCAAGUAACCAAGAA | 820 |
| | GCACG | | UAGG | |
| miR-26a-2-3p | CCUAUUCUUGAUUACUU | 821 | GAAACAAGUAAUCAAGA | 822 |
| | GUUUC | | AUAGG | |
| miR-26a-5p | UUCAAGUAAUCCAGGAU | 823 | AGCCUAUCCUGGAUUACU | 824 |
| | AGGCU | | UGAA | |
| miR-26b-3p | CCUGUUCUCCAUUACUU | 825 | GAGCCAAGUAAUGGAGA | 826 |
| | GGCUC | | ACAGG | |
| miR-26b-5p | UUCAAGUAAUUCAGGAU | 827 | ACCUAUCCUGAAUUACUU | 828 |
| | AGGU | | GAA | |
| miR-27a-3p | UUCACAGUGGCUAAGUU | 829 | GCGGAACUUAGCCACUGU | 830 |
| | CCGC | | GAA | |
| miR-27a-5p | AGGGCUUAGCUGCUUGU | 831 | UGCUCACAAGCAGCUAAG | 832 |
| | GAGCA | | CCCU | |
| miR-27b-3p | UUCACAGUGGCUAAGUU | 833 | GCAGAACUUAGCCACUGU | 834 |
| | CUGC | | GAA | |
| miR-27b-5p | AGAGCUUAGCUGAUUGG | 835 | GUUCACCAAUCAGCUAAG | 836 |
| | UGAAC | | CUCU | |
| miR-28-3p | CACUAGAUUGUGAGCUC | 837 | UCCAGGAGCUCACAAUCU | 838 |
| | CUGGA | | AGUG | |
| miR-28-5p | AAGGAGCUCACAGUCUA | 839 | CUCAAUAGACUGUGAGCU | 840 |
| | UUGAG | | CCUU | |
| miR-2861 | GGGGCCUGGCGGUGGGC | 841 | CCGCCCACCGCCAGGCCC | 842 |
| | GG | | C | |
| miR-2909 | GUUAGGGCCAACAUCUC | 843 | CCAAGAGAUGUUGGCCCU | 844 |
| | UUGG | | AAC | |
| miR-296-3p | GAGGGUUGGGUGGAGGC | 845 | GGAGAGCCUCCACCCAAC | 846 |
| | UCUCC | | CCUC | |
| miR-296-5p | AGGGCCCCCCCUCAAUC | 847 | ACAGGAUUGAGGGGGGG | 848 |
| | CUGU | | CCCU | |
| miR-2964a-3p | AGAAUUGCGUUUGGACA | 849 | ACUGAUUGUCCAAACGCA | 850 |
| | AUCAGU | | AUUCU | |
| miR-2964a-5p | AGAUGUCCAGCCACAAU | 851 | CGAGAAUUGUGGCUGGAC | 852 |
| | UCUCG | | AUCU | |
| miR-297 | AUGUAUGUGUGCAUGUG | 853 | CAUGCACAUGCACACAUA | 854 |
| | CAUG | | CAU | |
| miR-298 | AGCAGAAGCAGGGAGGU | 855 | UGGGAGAACCUCCCUGCU | 856 |
| | UCUCCCA | | UCUGCU | |
| miR-299-3p | UAUGUGGGAUGGUAAAC | 857 | AAGCGGUUUACCAUCCCA | 858 |
| | CGCUU | | CAUA | |
| miR-299-5p | UGGUUUACCGUCCCACA | 859 | AUGUAUGUGGGACGGUA | 860 |
| | UACAU | | AACCA | |
| miR-29a-3p | UAGCACCAUCUGAAAUC | 861 | UAACCGAUUUCAGAUGGU | 862 |
| | GGUUA | | GCUA | |
| miR-29a-5p | ACUGAUUUCUUUUGGUG | 863 | CUGAACACCAAAAGAAAU | 864 |
| | UUCAG | | CAGU | |
| miR-29b-1-5p | GCUGGUUUCAUAUGGUG | 865 | UCUAAACCACCAUAUGAA | 866 |
| | GUUUAGA | | ACCAGC | |
| miR-29b-2-5p | CUGGUUUCACAUGGUGG | 867 | CUAAGCCACCAUGUGAAA | 868 |
| | CUUAG | | CCAG | |
| miR-29b-3p | UAGCACCAUUUGAAAUC | 869 | AACACUGAUUUCAAAUGG | 870 |
| | AGUGUU | | UGCUA | |
| miR-29c-3p | UAGCACCAUUUGAAAUC | 871 | UAACCGAUUUCAAAUGGU | 872 |
| | GGUUA | | GCUA | |
| miR-29c-5p | UGACCGAUUUCUCCUGG | 873 | GAACACCAGGAGAAAUCG | 874 |
| | UGUUC | | GUCA | |
| miR-300 | UAUACAAGGGCAGACUC | 875 | AGAGAGAGUCUGCCCUUG | 876 |
| | UCUCU | | UAUA | |
| miR-301a-3p | CAGUGCAAUAGUAUUGU | 877 | GCUUUGACAAUACUAUUG | 878 |
| | CAAAGC | | CACUG | |
| miR-301a-5p | GCUCUGACUUUAUUGCA | 879 | AGUAGUGCAAUAAAGUC | 880 |
| | CUACU | | AGAGC | |
| miR-301b | CAGUGCAAUGAUAUUGU | 881 | GCUUUGACAAUAUCAUUG | 882 |
| | CAAAGC | | CACUG | |
| miR-302a-3p | UAAGUGCUUCCAUGUUU | 883 | UCACCAAAACAUGGAAGC | 884 |
| | UGGUGA | | ACUUA | |
| miR-302a-5p | ACUUAAACGUGGAUGUA | 885 | AGCAAGUACAUCCACGUU | 886 |
| | CUUGCU | | UAAGU | |
| miR-302b-3p | UAAGUGCUUCCAUGUUU | 887 | CUACUAAAACAUGGAAGC | 888 |
| | UAGUAG | | ACUUA | |
| miR-302b-5p | ACUUUAACAUGGAAGUG | 889 | GAAAGCACUUCCAUGUUA | 890 |
| | CUUUC | | AAGU | |
| miR-302c-3p | UAAGUGCUUCCAUGUUU | 891 | CCACUGAAACAUGGAAGC | 892 |
| | CAGUGG | | ACUUA | |
| miR-302c-5p | UUUAACAUGGGGGUACC | 893 | CAGCAGGUACCCCCAUGU | 894 |
| | UGCUG | | UAAA | |
| miR-302d-3p | UAAGUGCUUCCAUGUUU | 895 | ACACUCAAACAUGGAAGC | 896 |
| | GAGUGU | | ACUUA | |
| miR-302d-5p | ACUUUAACAUGGAGGCA | 897 | GCAAGUGCCUCCAUGUUA | 898 |
| | CUUGC | | AAGU | |
| miR-302e | UAAGUGCUUCCAUGCUU | 899 | AAGCAUGGAAGCACUUA | 900 |
| miR-302f | UAAUUGCUUCCAUGUUU | 901 | AAACAUGGAAGCAAUUA | 902 |
| miR-3064-3p | UUGCCACACUGCAACAC | 903 | UGUAAGGUGUUGCAGUG | 904 |
| | CUUACA | | UGGCAA | |
| miR-3064-5p | UCUGGCUGUUGUGGUGU | 905 | UUGCACACCACAACAGCC | 906 |
| | GCAA | | AGA | |
| miR-3065-3p | UCAGCACCAGGAUAUUG | 907 | CUCCAACAAUAUCCUGGU | 908 |
| | UUGGAG | | GCUGA | |
| miR-3065-5p | UCAACAAAAUCACUGAU | 909 | UCCAGCAUCAGUGAUUUU | 910 |
| | GCUGGA | | GUUGA | |
| miR-3074-3p | GAUAUCAGCUCAGUAGG | 911 | CGGUGCCUACUGAGCUGA | 912 |
| | CACCG | | UAUC | |
| miR-3074-5p | GUUCCUGCUGAACUGAG | 913 | CUGGCUCAGUUCAGCAGG | 914 |
| | CCAG | | AAC | |
| miR-30a-3p | CUUUCAGUCGGAUGUUU | 915 | GCUGCAAACAUCCGACUG | 916 |
| | GCAGC | | AAAG | |
| miR-30a-5p | UGUAAACAUCCUCGACU | 917 | CUUCCAGUCGAGGAUGUU | 918 |
| | GGAAG | | UACA | |
| miR-30b-3p | CUGGGAGGUGGAUGUUU | 919 | GAAGUAAACAUCCACCUC | 920 |
| | ACUUC | | CCAG | |
| miR-30b-5p | UGUAAACAUCCUACACU | 921 | AGCUGAGUGUAGGAUGU | 922 |
| | CAGCU | | UUACA | |
| miR-30c-1-3p | CUGGGAGAGGGUUGUUU | 923 | GGAGUAAACAACCCUCUC | 924 |
| | ACUCC | | CCAG | |
| miR-30c-2-3p | CUGGGAGAAGGCUGUUU | 925 | AGAGUAAACAGCCUUCUC | 926 |
| | ACUCU | | CCAG | |
| miR-30c-5p | UGUAAACAUCCUACACU | 927 | GCUGAGAGUGUAGGAUG | 928 |
| | CUCAGC | | UUUACA | |
| miR-30d-3p | CUUUCAGUCAGAUGUUU | 929 | GCAGCAAACAUCUGACUG | 930 |
| | GCUGC | | AAAG | |
| miR-30d-5p | UGUAAACAUCCCCGACU | 931 | CUUCCAGUCGGGGAUGUU | 932 |
| | GGAAG | | UACA | |
| miR-30e-3p | CUUUCAGUCGGAUGUUU | 933 | GCUGUAAACAUCCGACUG | 934 |
| | ACAGC | | AAAG | |
| miR-30e-5p | UGUAAACAUCCUUGACU | 935 | CUUCCAGUCAAGGAUGUU | 936 |
| | GGAAG | | UACA | |
| miR-31-3p | UGCUAUGCCAACAUAUU | 937 | AUGGCAAUAUGUUGGCA | 938 |
| | GCCAU | | UAGCA | |
| miR-31-5p | AGGCAAGAUGCUGGCAU | 939 | AGCUAUGCCAGCAUCUUG | 940 |
| | AGCU | | CCU | |
| miR-3115 | AUAUGGGUUUACUAGUU | 941 | ACCAACUAGUAAACCCAU | 942 |
| | GGU | | AU | |
| miR-3116 | UGCCUGGAACAUAGUAG | 943 | AGUCCCUACUAUGUUCCA | 944 |
| | GGACU | | GGCA | |
| miR-3117-3p | AUAGGACUCAUAUAGUG | 945 | CUGGCACUAUAUGAGUCC | 946 |
| | CCAG | | UAU | |
| miR-3117-5p | AGACACUAUACGAGUCA | 947 | AUAUGACUCGUAUAGUG | 948 |
| | UAU | | UCU | |
| miR-3118 | UGUGACUGCAUUAUGAA | 949 | AGAAUUUUCAUAAUGCA | 950 |
| | AAUUCU | | GUCACA | |
| miR-3119 | UGGCUUUUAACUUUGAU | 951 | GCCAUCAAAGUUAAAAGC | 952 |
| | GGC | | CA | |
| miR-3120-3p | CACAGCAAGUGUAGACA | 953 | UGCCUGUCUACACUUGCU | 954 |
| | GGCA | | GUG | |
| miR-3120-5p | CCUGUCUGUGCCUGCUG | 955 | UGUACAGCAGGCACAGAC | 956 |
| | UACA | | AGG | |
| miR-3121-3p | UAAAUAGAGUAGGCAAA | 957 | UGUCCUUUGCCUACUCUA | 958 |
| | GGACA | | UUUA | |
| miR-3121-5p | UCCUUUGCCUAUUCUAU | 959 | CUUAAAUAGAAUAGGCA | 960 |
| | UUAAG | | AAGGA | |
| miR-3122 | GUUGGGACAAGAGGACG | 961 | AAGACCGUCCUCUUGUCC | 962 |
| | GUCUU | | CAAC | |
| miR-3123 | CAGAGAAUUGUUUAAUC | 963 | GAUUAAACAAUUCUCUG | 964 |
| miR-3124-3p | ACUUUCCUCACUCCCGU | 965 | ACUUCACGGGAGUGAGGA | 966 |
| | GAAGU | | AAGU | |
| miR-3124-5p | UUCGCGGGCGAAGGCAA | 967 | GACUUUGCCUUCGCCCGC | 968 |
| | AGUC | | GAA | |
| miR-3125 | UAGAGGAAGCUGUGGAG | 969 | UCUCUCCACAGCUUCCUC | 970 |
| | AGA | | UA | |
| miR-3126-3p | CAUCUGGCAUCCGUCAC | 971 | UCUGUGUGACGGAUGCCA | 972 |
| | ACAGA | | GAUG | |
| miR-3126-5p | UGAGGGACAGAUGCCAG | 973 | UGCUUCUGGCAUCUGUCC | 974 |
| | AAGCA | | CUCA | |
| miR-3127-3p | UCCCCUUCUGCAGGCCU | 975 | CCAGCAGGCCUGCAGAAG | 976 |
| | GCUGG | | GGGA | |
| miR-3127-5p | AUCAGGGCUUGUGGAAU | 977 | CUUCCCAUUCCACAAGCC | 978 |
| | GGGAAG | | CUGAU | |
| miR-3128 | UCUGGCAAGUAAAAAAC | 979 | AUGAGAGUUUUUUACUU | 980 |
| | UCUCAU | | GCCAGA | |
| miR-3129-3p | AAAGCCUAAUCUCUACACU | 981 | GCAGCAGUGUAGAGAUU | 982 |
| | GCUGC | | AGUUU | |
| miR-3129-5p | GCAGUAGUGUAGAGAUU | 983 | AAACCAAUCUCUACACUA | 984 |
| | GGUUU | | CUGC | |
| miR-3130-3p | GCUGCACCGGAGACUGG | 985 | UUACCCAGUCUCCGGUGC | 986 |
| | GUAA | | AGC | |
| miR-3130-5p | UACCCAGUCUCCGGUGC | 987 | GGCUGCACCGGAGACUGG | 988 |
| | AGCC | | GUA | |
| miR-3131 | UCGAGGACUGGUGGAAG | 989 | AAGGCCCUUCCACCAGUC | 990 |
| | GGCCUU | | CUCGA | |
| miR-3132 | UGGGUAGAGAAGGAGCU | 991 | UCCUCUGAGCUCCUUCUC | 992 |
| | CAGAGGA | | UACCCA | |
| miR-3133 | UAAAGAACUCUUAAAAC | 993 | AUUGGGUUUUAAGAGUU | 994 |
| | CCAAU | | CUUUA | |
| miR-3134 | UGAUGGAUAAAAGACUA | 995 | AAUAUGUAGUCUUUUAU | 996 |
| | CAUAUU | | CCAUCA | |
| miR-3135a | UGCCUAGGCUGAGACUG | 997 | CACUGCAGUCUCAGCCUA | 998 |
| | CAGUG | | GGCA | |
| miR-3135b | GGCUGGAGCGAGUGCAG | 999 | CACCACUGCACUCGCUCC | 1000 |
| | UGGUG | | AGCC | |
| miR-3136-3p | UGGCCCAACCUAUUCAG | 1001 | ACUAACUGAAUAGGUUG | 1002 |
| | UUAGU | | GGCCA | |
| miR-3136-5p | CUGACUGAAUAGGUAGG | 1003 | AAUGACCCUACCUAUUCA | 1004 |
| | GUCAUU | | GUCAG | |
| miR-3137 | UCUGUAGCCUGGGAGCA | 1005 | ACCCCAUUGCUCCCAGGC | 1006 |
| | AUGGGGU | | UACAGA | |
| miR-3138 | UGUGGACAGUGAGGUAG | 1007 | ACUCCCUCUACCUCACUG | 1008 |
| | AGGGAGU | | UCCACA | |
| miR-3139 | UAGGAGCUCAACAGAUG | 1009 | AACAGGCAUCUGUUGAGC | 1010 |
| | CCUGUU | | UCCUA | |
| miR-3140-3p | AGCUUUUGGGAAUUCAG | 1011 | ACUACCUGAAUUCCCAAA | 1012 |
| | GUAGU | | AGCU | |
| miR-3140-5p | ACCUGAAUUACCAAAAG | 1013 | AAAGCUUUUGGUAAUUC | 1014 |
| | CUUU | | AGGU | |
| miR-3141 | GAGGGCGGGUGGAGGAG | 1015 | UCCUCCUCCACCCGCCCU | 1016 |
| | GA | | C | |
| miR-3142 | AAGGCCUUUCUGAACCU | 1017 | UCUGAAGGUUCAGAAAG | 1018 |
| | UCAGA | | GCCUU | |
| miR-3143 | AUAACAUUGUAAAGCGC | 1019 | CGAAAGAAGCGCUUUACA | 1020 |
| | UUCUUUCG | | AUGUUAU | |
| miR-3144-3p | AUAUACCUGUUCGGUCU | 1021 | UAAAGAGACCGAACAGGU | 1022 |
| | CUUUA | | AUAU | |
| miR-3144-5p | AGGGGACCAAAGAGAUA | 1023 | CUAUAUAUCUCUUUGGUC | 1024 |
| | UAUAG | | CCCU | |
| miR-3145-3p | AGAUAUUUUGAGUGUUU | 1025 | CAAUUCCAAACACUCAAA | 1026 |
| | GGAAUUG | | AUAUCU | |
| miR-3145-5p | AACUCCAAACACUCAAA | 1027 | UGAGUUUUGAGUGUUUG | 1028 |
| | ACUCA | | GAGUU | |
| miR-3146 | CAUGCUAGGAUAGAAAG | 1029 | CCAUUCUUUCUAUCCUAG | 1030 |
| | AAUGG | | CAUG | |
| miR-3147 | GGUUGGGCAGUGAGGAG | 1031 | UCACACCCUCCUCACUGC | 1032 |
| | GGUGUGA | | CCAACC | |
| miR-3148 | UGGAAAAAACUGGUGUG | 1033 | AAGCACACACCAGUUUUU | 1034 |
| | UGCUU | | UCCA | |
| miR-3149 | UUUGUAUGGAUAUGUGU | 1035 | AUACACACACAUAUCCAU | 1036 |
| | GUGUAU | | ACAAA | |
| miR-3150a-3p | CUGGGGAGAUCCUCGAG | 1037 | CCAACCUCGAGGAUCUCC | 1038 |
| | GUUGG | | CCAG | |
| miR-3150a-5p | CAACCUCGACGAUCUCC | 1039 | GCUGAGGAGAUCGUCGAG | 1040 |
| | UCAGC | | GUUG | |
| miR-3150b-3p | UGAGGAGAUCGUCGAGG | 1041 | CCAACCUCGACGAUCUCC | 1042 |
| | UUGG | | UCA | |
| miR-3150b-5p | CAACCUCGAGGAUCUCC | 1043 | GCUGGGGAGAUCCUCGAG | 1044 |
| | CCAGC | | GUUG | |
| miR-3151 | GGUGGGGCAAUGGGAUC | 1045 | ACCUGAUCCCAUUGCCCC | 1046 |
| | AGGU | | ACC | |
| miR-3152-3p | UGUGUUAGAAUAGGGGC | 1047 | UUAUUGCCCCUAUUCUAA | 1048 |
| | AAUAA | | CACA | |
| miR-3152-5p | AUUGCCUCUGUUCUAAC | 1049 | CUUGUGUUAGAACAGAG | 1050 |
| | ACAAG | | GCAAU | |
| miR-3153 | GGGGAAAGCGAGUAGGG | 1051 | AAAUGUCCCUACUCGCUU | 1052 |
| | ACAUUU | | UCCCC | |
| miR-3154 | CAGAAGGGGAGUUGGGA | 1053 | UCUGCUCCCAACUCCCCU | 1054 |
| | GCAGA | | UCUG | |
| miR-3155a | CCAGGCUCUGCAGUGGG | 1055 | AGUUCCCACUGCAGAGCC | 1056 |
| | AACU | | UGG | |
| miR-3155b | CCAGGCUCUGCAGUGGG | 1057 | UCCCACUGCAGAGCCUGG | 1058 |
| | A | | | |
| miR-3156-3p | CUCCCACUUCCAGAUCU | 1059 | AGAAAGAUCUGGAAGUG | 1060 |
| | UUCU | | GGAG | |
| miR-3156-5p | AAAGAUCUGGAAGUGGG | 1061 | UGUCUCCCACUUCCAGAU | 1062 |
| | AGACA | | CUUU | |
| miR-3157-3p | CUGCCCUAGUCUAGCUG | 1063 | AGCUUCAGCUAGACUAGG | 1064 |
| | AAGCU | | GCAG | |
| miR-3157-5p | UUCAGCCAGGCUAGUGC | 1065 | AGACUGCACUAGCCUGGC | 1066 |
| | AGUCU | | UGAA | |
| miR-3158-3p | AAGGGCUUCCUCUCUGC | 1067 | GUCCUGCAGAGAGGAAGC | 1068 |
| | AGGAC | | CCUU | |
| miR-3158-5p | CCUGCAGAGAGGAAGCC | 1069 | GAAGGGCUUCCUCUCUGC | 1070 |
| | CUUC | | AGG | |
| miR-3159 | UAGGAUUACAAGUGUCG | 1071 | GUGGCCGACACUUGUAAU | 1072 |
| | GCCAC | | CCUA | |
| miR-3160-3p | AGAGCUGAGACUAGAAA | 1073 | UGGGCUUUCUAGUCUCAG | 1074 |
| | GCCCA | | CUCU | |
| miR-3160-5p | GGCUUUCUAGUCUCAGC | 1075 | GGAGAGCUGAGACUAGA | 1076 |
| | UCUCC | | AAGAC | |
| miR-3161 | CUGAUAAGAACAGAGGC | 1077 | AUCUGGGCCUCUGUUCUU | 1078 |
| | CCAGAU | | AUCAG | |
| miR-3162-3p | UCCCUACCCCUCCACUCC | 1079 | UGGGGAGUGGAGGGGUA | 1080 |
| | CCA | | GGGA | |
| miR-3162-5p | UUAGGGAGUAGAAGGGU | 1081 | CUCCCCACCCUUCUACUC | 1082 |
| | GGGGAG | | CCUAA | |
| miR-3163 | UAUAAAAUGAGGGCAGU | 1083 | GUCUUACUGCCCUCAUUU | 1084 |
| | AAGAC | | UAUA | |
| miR-3164 | UGUGACUUUAAGGGAAA | 1085 | CGCCAUUUCCCUUAAAGU | 1086 |
| | UGGCG | | CACA | |
| miR-3165 | AGGUGGAUGCAAUGUGA | 1087 | UGAGGUCACAUUGCAUCC | 1088 |
| | CCUCA | | ACCU | |
| miR-3166 | CGCAGACAAUGCCUACU | 1089 | UAGGCCAGUAGGCAUUGU | 1090 |
| | GGCCUA | | CUGCG | |
| miR-3167 | AGGAUUUCAGAAAUACU | 1091 | ACACCAGUAUUUCUGAAA | 1092 |
| | GGUGU | | UCCU | |
| miR-3168 | GAGUUCUACAGUCAGAC | 1093 | GUCUGACUGUAGAACUC | 1094 |
| miR-3169 | UAGGACUGUGCUUGGCA | 1095 | CUAUGUGCCAAGCACAGU | 1096 |
| | CAUAG | | CCUA | |
| miR-3170 | CUGGGGUUCUGAGACAG | 1097 | ACUGUCUGUCUCAGAACC | 1098 |
| | ACAGU | | CCAG | |
| miR-3171 | AGAUGUAUGGAAUCUGU | 1099 | GAUAUAUACAGAUUCCAU | 1100 |
| | AUAUAUC | | ACAUCU | |
| miR-3173-3p | AAAGGAGGAAAUAGGCA | 1101 | UGGCCUGCCUAUUUCCUC | 1102 |
| | GGCCA | | CUUU | |
| miR-3173-5p | UGCCCUGCCUGUUUUCU | 1103 | AAAGGAGAAAACAGGCA | 1104 |
| | CCUUU | | GGGCA | |
| miR-3174 | UAGUGAGUUAGAGAUGC | 1105 | GGCUCUGCAUCUCUAACU | 1106 |
| | AGAGCC | | CACUA | |
| miR-3175 | CGGGGAGAGAACGCAGU | 1107 | ACGUCACUGCGUUCUCUC | 1108 |
| | GACGU | | CCCG | |
| miR-3176 | ACUGGCCUGGGACUACC | 1109 | CCGGUAGUCCCAGGCCAG | 1110 |
| | GG | | U | |
| miR-3177-3p | UGCACGGCACUGGGGAC | 1111 | ACGUGUCCCCAGUGCCGU | 1112 |
| | ACGU | | GCA | |
| miR-3177-5p | UGUGUACACACGUGCCA | 1113 | AGCGCCUGGCACGUGUGU | 1114 |
| | GGCGCU | | ACACA | |
| miR-3178 | GGGGCGCGGCCGGAUCG | 1115 | CGAUCCGGCCGCGCCCC | 1116 |
| miR-3179 | AGAAGGGGUGAAAUUUA | 1117 | ACGUUUAAAUUUCACCCC | 1118 |
| | AACGU | | UUCU | |
| miR-3180 | UGGGGCGGAGCUUCCGG | 1119 | CUCCGGAAGCUCCGCCCC | 1120 |
| | AG | | A | |
| miR-3180-3p | UGGGGCGGAGCUUCCGG | 1121 | GGCCUCCGGAAGCUCCGC | 1122 |
| | AGGCC | | CCCA | |
| miR-3180-5p | CUUCCAGACGCUCCGCC | 1123 | CGACGUGGGGCGGAGCGU | 1124 |
| | CCACGUCG | | CUGGAAG | |
| miR-3181 | AUCGGGCCCUCGGCGCC | 1125 | CCGGCGCCGAGGGCCCGA | 1126 |
| | GG | | U | |
| miR-3182 | GCUUCUGUAGUGUAGUC | 1127 | GACUACACUACAGAAGC | 1128 |
| miR-3183 | GCCUCUCUCGGAGUCGC | 1129 | UCCGAGCGACUCCGAGAG | 1130 |
| | UCGGA | | AGGC | |
| miR-3184-3p | AAAGUCUCGCUCUCUGC | 1131 | UGAGGGGCAGAGAGCGA | 1132 |
| | CCCUCA | | GACUUU | |
| miR-3184-5p | UGAGGGGCCUCAGACCG | 1133 | AAAAGCUCGGUCUGAGGC | 1134 |
| | AGCUUUU | | CCCUCA | |
| miR-3185 | AGAAGAAGGCGGUCGGU | 1135 | CCGCAGACCGACCGCCUU | 1136 |
| | CUGCGG | | CUUCU | |
| miR-3186-3p | UCACGCGGAGAGAUGGC | 1137 | CAAAGCCAUCUCUCCGCG | 1138 |
| | UUUG | | UGA | |
| miR-3186-5p | CAGGCGUCUGUCUACGU | 1139 | AAGCCACGUAGACAGACG | 1140 |
| | GGCUU | | CCUG | |
| miR-3187-3p | UUGGCCAUGGGGCUGCG | 1141 | CCGCGCAGCCCCAUGGCC | 1142 |
| | CGG | | AA | |
| miR-3187-5p | CCUGGGCAGCGUGUGGC | 1143 | CCUUCAGCCACACGCUGC | 1144 |
| | UGAAGG | | CCAGG | |
| miR-3188 | AGAGGCUUUGUGCGGAU | 1145 | CCCCGUAUCCGCACAAAG | 1146 |
| | ACGGGG | | CCUCU | |
| miR-3189-3p | CCCUUGGGUCUGAUGGG | 1147 | CUACCCCAUCAGACCCAA | 1148 |
| | GUAG | | GGG | |
| miR-3189-5p | UGCCCCAUCUGUGCCCU | 1149 | UCCUACCCAGGGCACAGA | 1150 |
| | GGGUAGGA | | UGGGGCA | |
| miR-3190-3p | UGUGGAAGGUAGACGGC | 1151 | UCUCUGGCCGUCUACCUU | 1152 |
| | CAGAGA | | CCACA | |
| miR-3190-5p | UCUGGCCAGCUACGUCC | 1153 | UGGGGACGUAGCUGGCCA | 1154 |
| | CCA | | GA | |
| miR-3191-3p | UGGGGACGUAGCUGGCC | 1155 | CUGUCUGGCCAGCUACGU | 1156 |
| | AGACAG | | CCCCA | |
| miR-3191-5p | CUCUCUGGCCGUCUACC | 1157 | UGGAAGGUAGACGGCCAG | 1158 |
| | UUCCA | | AGAG | |
| miR-3192 | UCUGGGAGGUUGUAGCA | 1159 | UUCCACUGCUACAACCUC | 1160 |
| | GUGGAA | | CCAGA | |
| miR-3193 | UCCUGCGUAGGAUCUGA | 1161 | ACUCCUCAGAUCCUACGC | 1162 |
| | GGAGU | | AGGA | |
| miR-3194-3p | AGCUCUGCUGCUCACUG | 1163 | ACUGCCAGUGAGCAGCAG | 1164 |
| | GCAGU | | AGCU | |
| miR-3194-5p | GGCCAGCCACCAGGAGG | 1165 | CAGCCCUCCUGGUGGCUG | 1166 |
| | GCUG | | GCC | |
| miR-3195 | CGCGCCGGGCCCGGGUU | 1167 | AACCCGGGCCCGGCGCG | 1168 |
| miR-3196 | CGGGGCGGCAGGGGCCU | 1169 | GAGGCCCCUGCCGCCCCG | 1170 |
| | C | | | |
| miR-3197 | GGAGGCGCAGGCUCGGA | 1171 | CGCCUUUCCGAGCCUGCG | 1172 |
| | AAGGCG | | CCUCC | |
| miR-3198 | GUGGAGUCCUGGGGAAU | 1173 | UCUCCAUUCCCCAGGACU | 1174 |
| | GGAGA | | CCAC | |
| miR-3199 | AGGGACUGCCUUAGGAG | 1175 | AACUUUCUCCUAAGGCAG | 1176 |
| | AAAGUU | | UCCCU | |
| miR-32-3p | CAAUUUAGUGUGUGUGA | 1177 | AAAUAUCACACACACUAA | 1178 |
| | UAUUU | | AUUG | |
| miR-32-5p | UAUUGCACAUUACUAAG | 1179 | UGCAACUUAGUAAUGUGC | 1180 |
| | UUGCA | | AAUA | |
| miR-3200-3p | CACCUUGCGCUACUCAG | 1181 | CAGACCUGAGUAGCGCAA | 1182 |
| | GUCUG | | GGUG | |
| miR-3200-5p | AAUCUGAGAAGGCGCAC | 1183 | ACCUUGUGCGCCUUCUCA | 1184 |
| | AAGGU | | GAUU | |
| miR-3201 | GGGAUAUGAAGAAAAAU | 1185 | AUUUUUCUUCAUAUCCC | 1186 |
| miR-3202 | UGGAAGGGAGAAGAGCU | 1187 | AUUAAAGCUCUUCUCCCU | 1188 |
| | UUAAU | | UCCA | |
| miR-320a | AAAAGCUGGGUUGAGAG | 1189 | UCGCCCUCUCAACCCAGC | 1190 |
| | GGCGA | | UUUU | |
| miR-320b | AAAAGCUGGGUUGAGAG | 1191 | UCGCCCUCUCAACCCAGC | 1192 |
| | GGCAA | | UUUU | |
| miR-320c | AAAAGCUGGGUUGAGAG | 1193 | ACCCUCUCAACCCAGCUU | 1194 |
| | GGU | | UU | |
| miR-320d | AAAAGCUGGGUUGAGAG | 1195 | UCCUCUCAACCCAGCUUU | 1196 |
| | GA | | U | |
| miR-320e | AAAGCUGGGUUGAGAAG | 1197 | CCUUCUCAACCCAGCUUU | 1198 |
| | G | | | |
| miR-323a-3p | CACAUUACACGGUCGAC | 1199 | AGAGGUCGACCGUGUAAU | 1200 |
| | CUCU | | GUG | |
| miR-323a-5p | AGGUGGUCCGUGGCGCG | 1201 | GCGAACGCGCCACGGACC | 1202 |
| | UUCGC | | ACCU | |
| miR-323b-3p | CCCAAUACACGGUCGAC | 1203 | AAGAGGUCGACCGUGUAU | 1204 |
| | CUCUU | | UGGG | |
| miR-323b-5p | AGGUUGUCCGUGGUGAG | 1205 | UGCGAACUCACCACGGAC | 1206 |
| | UUCGCA | | AACCU | |
| miR-324-3p | ACUGCCCCAGGUGCUGC | 1207 | CCAGCAGCACCUGGGGCA | 1208 |
| | UGG | | GU | |
| miR-324-5p | CGCAUCCCCUAGGGCAU | 1209 | ACACCAAUGCCCUAGGGG | 1210 |
| | UGGUGU | | AUGCG | |
| miR-325 | CCUAGUAGGUGUCCAGU | 1211 | ACACUUACUGGACACCUA | 1212 |
| | AAGUGU | | CUAGG | |
| miR-326 | CCUCUGGGCCCUUCCUC | 1213 | CUGGAGGAAGGGCCCAGA | 1214 |
| | CAG | | GG | |
| miR-328 | CUGGCCCUCUCUGCCCU | 1215 | ACGGAAGGGCAGAGAGG | 1216 |
| | UCCGU | | GCCAG | |
| miR-329 | AACACACCUGGUUAACC | 1217 | AAAGAGGUUAACCAGGU | 1218 |
| | UCUUU | | GUGUU | |
| miR-330-3p | GCAAAGCACACGGCCUG | 1219 | UCUCUGCAGGCCGUGUGC | 1220 |
| | CAGAGA | | UUUGC | |
| miR-330-5p | UCUCUGGGCCUGUGUCU | 1221 | GCCUAAGACACAGGCCCA | 1222 |
| | UAGGC | | GAGA | |
| miR-331-3p | GCCCCUGGGCCUAUCCU | 1223 | UUCUAGGAUAGGCCCAGG | 1224 |
| | AGAA | | GGC | |
| miR-331-5p | CUAGGUAUGGUCCCAGG | 1225 | GGAUCCCUGGGACCAUAC | 1226 |
| | GAUCC | | CUAG | |
| miR-335-3p | UUUUUCAUUAUUGCUCC | 1227 | GGUCAGGAGCAAUAAUG | 1228 |
| | UGACC | | AAAAA | |
| miR-335-5p | UCAAGAGCAAUAACGAA | 1229 | ACAUUUUUCGUUAUUGCU | 1230 |
| | AAAUGU | | CUUGA | |
| miR-337-3p | CUCCUAUAUGAUGCCUU | 1231 | GAAGAAAGGCAUCAUAU | 1232 |
| | UCUUC | | AGGAG | |
| miR-337-5p | GAACGGCUUCAUACAGG | 1233 | AACUCCUGUAUGAAGCCG | 1234 |
| | AGUU | | UUC | |
| miR-338-3p | UCCAGCAUCAGUGAUUU | 1235 | CAACAAAAUCACUGAUGC | 1236 |
| | UGUUG | | UGGA | |
| miR-338-5p | AACAAUAUCCUGGUGCU | 1237 | CACUCAGCACCAGGAUAU | 1238 |
| | GAGUG | | UGUU | |
| miR-339-3p | UGAGCGCCUCGACGACA | 1239 | CGGCUCUGUCGUCGAGGC | 1240 |
| | GAGCCG | | GCUCA | |
| miR-339-5p | UCCCUGUCCUCCAGGAG | 1241 | CGUGAGCUCCUGGAGGAC | 1242 |
| | CUCACG | | AGGGA | |
| miR-33a-3p | CAAUGUUUCCACAGUGC | 1243 | GUGAUGCACUGUGGAAAC | 1244 |
| | AUCAC | | AUUG | |
| miR-33a-5p | GUGCAUUGUAGUUGCAU | 1245 | UGCAAUGCAACUACAAUG | 1246 |
| | UGCA | | CAC | |
| miR-33b-3p | CAGUGCCUCGGCAGUGC | 1247 | GGGCUGCACUGCCGAGGC | 1248 |
| | AGCCC | | ACUG | |
| miR-33b-5p | GUGCAUUGCUGUUGCAU | 1249 | GCAAUGCAACAGCAAUGC | 1250 |
| | UGC | | AC | |
| miR-340-3p | UCCGUCUCAGUUACUUU | 1251 | GCUAUAAAGUAACUGAG | 1252 |
| | AUAGC | | ACGGA | |
| miR-340-5p | UUAUAAAGCAAUGAGAC | 1253 | AAUCAGUCUCAUUGCUUU | 1254 |
| | UGAUU | | AUAA | |
| miR-342-3p | UCUCACACAGAAAUCGC | 1255 | ACGGGUGCGAUUUCUGUG | 1256 |
| | ACCCGU | | UGAGA | |
| miR-342-5p | AGGGGUGCUAUCUGUGA | 1257 | UCAAUCACAGAUAGCACC | 1258 |
| | UUGA | | CCU | |
| miR-345-3p | GCCCUGAACGAGGGGUC | 1259 | CUCCAGACCCCUCGUUCA | 1260 |
| | UGGAG | | GGGC | |
| miR-345-5p | GCUGACUCCUAGUCCAG | 1261 | GAGCCCUGGACUAGGAGU | 1262 |
| | GGCUC | | CAGC | |
| miR-346 | UGUCUGCCCGCAUGCCU | 1263 | AGAGGCAGGCAUGCGGGC | 1264 |
| | GCCUCU | | AGACA | |
| miR-34a-3p | CAAUCAGCAAGUAUACU | 1265 | AGGGCAGUAUACUUGCUG | 1266 |
| | GCCCU | | AUUG | |
| miR-34a-5p | UGGCAGUGUCUUAGCUG | 1267 | ACAACCAGCUAAGACACU | 1268 |
| | GUUGU | | GCCA | |
| miR-34b-3p | CAAUCACUAACUCCACU | 1269 | AUGGCAGUGGAGUUAGU | 1270 |
| | GCCAU | | GAUUG | |
| miR-34b-5p | UAGGCAGUGUCAUUAGC | 1271 | CAAUCAGCUAAUGACACU | 1272 |
| | UGAUUG | | GCCUA | |
| miR-34c-3p | AAUCACUAACCACACGG | 1273 | CCUGGCCGUGUGGUUAGU | 1274 |
| | CCAGG | | GAUU | |
| miR-34c-5p | AGGCAGUGUAGUUAGCU | 1275 | GCAAUCAGCUAACUACAC | 1276 |
| | GAUUGC | | UGCCU | |
| miR-3529-3p | AACAACAAAAUCACUAG | 1277 | UGGAAGACUAGUGAUUU | 1278 |
| | UCUUCCA | | UGUUGUU | |
| miR-3529-5p | AGGUAGACUGGGAUUUG | 1279 | AACAACAAAUCCCAGUCU | 1280 |
| | UUGUU | | ACCU | |
| miR-3545-3p | UUGAACUGUUAAGAACC | 1281 | UCCAGUGGUUCUUAACAG | 1282 |
| | ACUGGA | | UUCAA | |
| miR-3545-5p | UAGUGGUCCUAAACAUU | 1283 | UGUGAAAUGUUUAGGAC | 1284 |
| | UCACA | | CACUA | |
| miR-3591-3p | CACCAUUGUCACAC | 1285 | GUGGAGUGUGACAAUGG | 1286 |
| | UCCAC | | UGUUU | |
| miR-3591-5p | UUUAGUGUGAUAAUGGC | 1287 | UCAAACGCCAUUAUCACA | 1288 |
| | GUUUGA | | CUAAA | |
| miR-3605-3p | CCUCCGUGUUACCUGUC | 1289 | CUAGAGGACAGGUAACAC | 1290 |
| | CUCUAG | | GGAGG | |
| miR-3605-5p | UGAGGAUGGAUAGCAAG | 1291 | GGCUUCCUUGCUAUCCAU | 1292 |
| | GAAGCC | | CCUCA | |
| miR-3606 | UUAGUGAAGGCUAUUUU | 1293 | AAUUAAAAUAGCCUUCAC | 1294 |
| | AAUU | | UAA | |
| miR-3607-3p | ACUGUAAACGCUUUCUG | 1295 | CAUCAGAAAGCGUUUACA | 1296 |
| | AUG | | GU | |
| miR-3607-5p | GCAUGUGAUGAAGCAAA | 1297 | ACUGAUUUGCUUCAUCAC | 1298 |
| | UCAGU | | AUGC | |
| miR-3609 | CAAAGUGAUGAGUAAUA | 1299 | CAGCCAGUAUUACUCAUC | 1300 |
| | CUGGCUG | | ACUUUG | |
| miR-361-3p | UCCCCCAGGUGUGAUUC | 1301 | AAAUCAGAAUCACACCUG | 1302 |
| | UGAUUU | | GGGGA | |
| miR-361-5p | UUAUCAGAAUCUCCAGG | 1303 | GUACCCCUGGAGAUUCUG | 1304 |
| | GGUAC | | AUAA | |
| miR-3610 | GAAUCGGAAAGGAGGCG | 1305 | CGGCGCCUCCUUUCCGAU | 1306 |
| | CCG | | UC | |
| miR-3611 | UUGUGAAGAAAGAAAUU | 1307 | UAAGAAUUUCUUUCUUCA | 1308 |
| | CUUA | | CAA | |
| miR-3612 | AGGAGGCAUCUUGAGAA | 1309 | UCCAUUUCUCAAGAUGCC | 1310 |
| | AUGGA | | UCCU | |
| miR-3613-3p | ACAAAAAAAAAAGCCCA | 1311 | GAAGGGUUGGGCUUUUU | 1312 |
| | ACCCUUC | | UUUUUGU | |
| miR-3613-5p | UGUUGUACUUUUUUUUU | 1313 | GAACAAAAAAAAAAGUA | 1314 |
| | UGUUC | | CAACA | |
| miR-3614-3p | UAGCCUUCAGAUCUUGG | 1315 | AAAACACCAAGAUCUGAA | 1316 |
| | UGUUUU | | GGCUA | |
| miR-3614-5p | CCACUUGGAUCUGAAGG | 1317 | GGGCAGCCUUCAGAUCCA | 1318 |
| | CUGCCC | | AGUGG | |
| miR-3615 | UCUCUCGGCUCCUCGCG | 1319 | GAGCCGCGAGGAGCCGAG | 1320 |
| | GCUC | | AGA | |
| miR-3616-3p | CGAGGGCAUUUCAUGAU | 1321 | GCCUGCAUCAUGAAAUGC | 1322 |
| | GCAGGC | | CCUCG | |
| miR-3616-5p | AUGAAGUGCACUCAUGA | 1323 | ACAUAUCAUGAGUGCACU | 1324 |
| | UAUGU | | UCAU | |
| miR-3617 | AAAGACAUAGUUGCAAG | 1325 | CCCAUCUUGCAACUAUGU | 1326 |
| | AUGGG | | CUUU | |
| miR-3618 | UGUCUACAUUAAUGAAA | 1327 | GCUCUUUUCAUUAAUGUA | 1328 |
| | AGAGC | | GACA | |
| miR-3619-3p | GGGACCAUCCUGCCUGC | 1329 | CCACAGCAGGCAGGAUGG | 1330 |
| | UGUGG | | UCCC | |
| miR-3619-5p | UCAGCAGGCAGGCUGGU | 1331 | GCUGCACCAGCCUGCCUG | 1332 |
| | GCAGC | | CUGA | |
| miR-362-3p | AACACACCUAUUCAAGG | 1333 | UGAAUCCUUGAAUAGGU | 1334 |
| | AUUCA | | GUGUU | |
| miR-362-5p | AAUCCUUGGAACCUAGG | 1335 | ACUCACACCUAGGUUCCA | 1336 |
| | UGUGAGU | | AGGAUU | |
| miR-3620 | UCACCCUGCAUCCCGCA | 1337 | CUGGGUGCGGGAUGCAGG | 1338 |
| | CCCAG | | GUGA | |
| miR-3621 | CGCGGGUCGGGGUCUGC | 1339 | CCUGCAGACCCCGACCCG | 1340 |
| | AGG | | CG | |
| miR-3622a-3p | UCACCUGACCUCCCAUG | 1341 | ACAGGCAUGGGAGGUCAG | 1342 |
| | CCUGU | | GUGA | |
| miR-3622a-5p | CAGGCACGGGAGCUCAG | 1343 | CUCACCUGAGCUCCCGUG | 1344 |
| | GUGAG | | CCUG | |
| miR-3622b-3p | UCACCUGAGCUCCCGUG | 1345 | CAGGCACGGGAGCUCAGG | 1346 |
| | CCUG | | UGA | |
| miR-3622b-5p | AGGCAUGGGAGGUCAGG | 1347 | UCACCUGACCUCCCAUGC | 1348 |
| | UGA | | CU | |
| miR-363-3p | AAUUGCACGGUAUCCAU | 1349 | UACAGAUGGAUACCGUGC | 1350 |
| | CUGUA | | AAUU | |
| miR-363-5p | CGGGUGGAUCACGAUGC | 1351 | AAAUUGCAUCGUGAUCCA | 1352 |
| | AAUUU | | CCCG | |
| miR-3646 | AAAAUGAAAUGAGCCCA | 1353 | UGGGCUGGGCUCAUUUCA | 1354 |
| | GCCCA | | UUUU | |
| miR-3648 | AGCCGCGGGGAUCGCCG | 1355 | CCCUCGGCGAUCCCCGCG | 1356 |
| | AGGG | | GCU | |
| miR-3649 | AGGGACCUGAGUGUCUA | 1357 | CUUAGACACUCAGGUCCC | 1358 |
| | AG | | U | |
| miR-3650 | AGGUGUGUCUGUAGAGU | 1359 | GGACUCUACAGACACACC | 1360 |
| | CC | | U | |
| miR-3651 | CAUAGCCCGGUCGCUGG | 1361 | UCAUGUACCAGCGACCGG | 1362 |
| | UACAUGA | | GCUAUG | |
| miR-3652 | CGGCUGGAGGUGUGAGG | 1363 | UCCUCACACCUCCAGCCG | 1364 |
| | A | | | |
| miR-3653 | CUAAGAAGUUGACUGAA | 1365 | CUUCAGUCAACUUCUUAG | 1366 |
| | G | | | |
| miR-3654 | GACUGGACAAGCUGAGG | 1367 | UUCCUCAGCUUGUCCAGU | 1368 |
| | AA | | C | |
| miR-3655 | GCUUGUCGCUGCGGUGU | 1369 | AGCAACACCGCAGCGACA | 1370 |
| | UGCU | | AGC | |
| miR-3656 | GGCGGGUGCGGGGGUGG | 1371 | CCACCCCCGCACCCGCC | 1372 |
| miR-3657 | UGUGUCCCAUUAUUGGU | 1373 | AAUCACCAAUAAUGGGAC | 1374 |
| | GAUU | | ACA | |
| miR-3658 | UUUAAGAAAACACCAUG | 1375 | AUCUCCAUGGUGUUUUCU | 1376 |
| | GAGAU | | UAAA | |
| miR-3659 | UGAGUGUUGUCUACGAG | 1377 | UGCCCUCGUAGACAACAC | 1378 |
| | GGCA | | UCA | |
| miR-365a-3p | UAAUGCCCCUAAAAAUC | 1379 | AUAAGGAUUUUUAGGGG | 1380 |
| | CUUAU | | CAUUA | |
| miR-365a-5p | AGGGACUUUUGGGGGCA | 1381 | CACAUCUGCCCCCAAAAG | 1382 |
| | GAUGUG | | UCCCU | |
| miR-365b-3p | UAAUGCCCCUAAAAAUC | 1383 | AUAAGGAUUUUUAGGGG | 1384 |
| | CUUAU | | CAUUA | |
| miR-365b-5p | AGGGACUUUCAGGGGCA | 1385 | ACAGCUGCCCCUGAAAGU | 1386 |
| | GCUGU | | CCCU | |
| miR-3660 | ACUGACAGGAGAGCAUU | 1387 | UCAAAAUGCUCUCCUGUC | 1388 |
| | UUGA | | AGU | |
| miR-3661 | UGACCUGGGACUCGGAC | 1389 | CAGCUGUCCGAGUCCCAG | 1390 |
| | AGCUG | | GUCA | |
| miR-3662 | GAAAAUGAUGAGUAGUG | 1391 | CAUCAGUCACUACUCAUC | 1392 |
| | ACUGAUG | | AUUUUC | |
| miR-3663-3p | UGAGCACCACACAGGCC | 1393 | GCGCCCGGCCUGUGUGGU | 1394 |
| | GGGCGC | | GCUCA | |
| miR-3663-5p | GCUGGUCUGCGUGGUGC | 1395 | CCGAGCACCACGCAGACC | 1396 |
| | UCGG | | AGC | |
| miR-3664-3p | UCUCAGGAGUAAAGACA | 1397 | AACUCUGUCUUUACUCCU | 1398 |
| | GAGUU | | GAGA | |
| miR-3664-5p | AACUCUGUCUUCACUCA | 1399 | ACUCAUGAGUGAAGACAG | 1400 |
| | UGAGU | | AGUU | |
| miR-3665 | AGCAGGUGCGGGGCGGC | 1401 | CGCCGCCCCGCACCUGCU | 1402 |
| | G | | | |
| miR-3666 | CAGUGCAAGUGUAGAUG | 1403 | UCGGCAUCUACACUUGCA | 1404 |
| | CCGA | | CUG | |
| miR-3667-3p | ACCUUCCUCUCCAUGGG | 1405 | AAAGACCCAUGGAGAGGA | 1406 |
| | UCUUU | | AGGU | |
| miR-3667-5p | AAAGACCCAUUGAGGAG | 1407 | ACCUUCUCCUCAAUGGGU | 1408 |
| | AAGGU | | CUUU | |
| miR-3668 | AAUGUAGAGAUUGAUCA | 1409 | AUUUUGAUCAAUCUCUAC | 1410 |
| | AAAU | | AUU | |
| miR-3669 | ACGGAAUAUGUAUACGG | 1411 | UAUAUUCCGUAUACAUAU | 1412 |
| | AAUAUA | | UCCGU | |
| miR-367-3p | AAUUGCACUUUAGCAAU | 1413 | UCACCAUUGCUAAAGUGC | 1414 |
| | GGUGA | | AAUU | |
| miR-367-5p | ACUGUUGCUAAUAUGCA | 1415 | AGAGUUGCAUAUUAGCA | 1416 |
| | ACUCU | | ACAGU | |
| miR-3670 | AGAGCUCACAGCUGUCC | 1417 | UAGAGAAGGACAGCUGU | 1418 |
| | UUCUCUA | | GAGCUCU | |
| miR-3671 | AUCAAAUAAGGACUAGU | 1419 | UGCAGACUAGUCCUUAUU | 1420 |
| | CUGCA | | UGAU | |
| miR-3672 | AUGAGACUCAUGUAAAA | 1421 | AAGAUGUUUUACAUGAG | 1422 |
| | CAUCUU | | UCUCAU | |
| miR-3673 | AUGGAAUGUAUAUACGG | 1423 | UAUUCCGUAUAUACAUUC | 1424 |
| | AAUA | | CAU | |
| miR-3674 | AUUGUAGAACCUAAGAU | 1425 | GGCCAAUCUUAGGUUCUA | 1426 |
| | UGGCC | | CAAU | |
| miR-3675-3p | CAUCUCUAAGGAACUCC | 1427 | UUGGGGGAGUUCCUUAG | 1428 |
| | CCCAA | | AGAUG | |
| miR-3675-5p | UAUGGGGCUUCUGUAGA | 1429 | GAAAUCUCUACAGAAGCC | 1430 |
| | GAUUUC | | CCAUA | |
| miR-3676-3p | CCGUGUUUCCCCCACGC | 1431 | AAAGCGUGGGGGAAACAC | 1432 |
| | UUU | | GG | |
| miR-3676-5p | AGGAGAUCCUGGGUU | 1433 | AACCCAGGAUCUCCU | 1434 |
| miR-3677-3p | CUCGUGGGCUCUGGCCA | 1435 | GGCCGUGGCCAGAGCCCA | 1436 |
| | CGGCC | | CGAG | |
| miR-3677-5p | CAGUGGCCAGAGCCCUG | 1437 | CACUGCAGGGCUCUGGCC | 1438 |
| | CAGUG | | ACUG | |
| miR-3678-3p | CUGCAGAGUUUGUACGG | 1439 | CCGGUCCGUACAAACUCU | 1440 |
| | ACCGG | | GCAG | |
| miR-3678-5p | UCCGUACAAACUCUGCU | 1441 | CACAGCAGAGUUUGUACG | 1442 |
| | GUG | | GA | |
| miR-3679-3p | CUUCCCCCCAGUAAUCU | 1443 | GAUGAAGAUUACUGGGG | 1444 |
| | UCAUC | | GGAAG | |
| miR-3679-5p | UGAGGAUAUGGCAGGGA | 1445 | UCCCCUUCCCUGCCAUAU | 1446 |
| | AGGGGA | | CCUCA | |
| miR-3680-3p | UUUUGCAUGACCCUGGG | 1447 | CCUACUCCCAGGGUCAUG | 1448 |
| | AGUAGG | | CAAAA | |
| miR-3680-5p | GACUCACUCACAGGAUU | 1449 | UGCACAAUCCUGUGAGUG | 1450 |
| | GUGCA | | AGUC | |
| miR-3681-3p | ACACAGUGCUUCAUCCA | 1451 | AGUAGUGGAUGAAGCAC | 1452 |
| | CUACU | | UGUGU | |
| miR-3681-5p | UAGUGGAUGAUGCACUC | 1453 | GCACAGAGUGCAUCAUCC | 1454 |
| | UGUGC | | ACUA | |
| miR-3682-3p | UGAUGAUACAGGUGGAG | 1455 | CUACCUCCACCUGUAUCA | 1456 |
| | GUAG | | UCA | |
| miR-3682-5p | CUACUUCUACCUGUGUU | 1457 | AUGAUAACACAGGUAGA | 1458 |
| | AUCAU | | AGUAG | |
| miR-3683 | UGCGACAUUGGAAGUAG | 1459 | UGAUACUACUUCCAAUGU | 1460 |
| | UAUCA | | CGCA | |
| miR-3684 | UUAGACCUAGUACACGU | 1461 | AAGGACGUGUACUAGGUC | 1462 |
| | CCUU | | UAA | |
| miR-3685 | UUUCCUACCCUACCUGA | 1463 | AGUCUUCAGGUAGGGUA | 1464 |
| | AGACU | | GGAAA | |
| miR-3686 | AUCUGUAAGAGAAAGUA | 1465 | UCAUUUACUUUCUCUUAC | 1466 |
| | AAUGA | | AGAU | |
| miR-3687 | CCCGGACAGGCGUUCGU | 1467 | ACGUCGCACGAACGCCUG | 1468 |
| | GCGACGU | | UCCGGG | |
| miR-3688-3p | UAUGGAAAGACUUUGCC | 1469 | AGAGUGGCAAAGUCUUUC | 1470 |
| | ACUCU | | CAUA | |
| miR-3688-5p | AGUGGCAAAGUCUUUCC | 1471 | AUAUGGAAAGACUUUGCC | 1472 |
| | AUAU | | ACU | |
| miR-3689a-3p | CUGGGAGGUGUGAUAUC | 1473 | ACCACGAUAUCACACCUC | 1474 |
| | GUGGU | | CCAG | |
| miR-3689a-5p | UGUGAUAUCAUGGUUCC | 1475 | UCCCAGGAACCAUGAUAU | 1476 |
| | UGGGA | | CACA | |
| miR-3689b-3p | CUGGGAGGUGUGAUAUU | 1477 | ACCACAAUAUCACACCUC | 1478 |
| | GUGGU | | CCAG | |
| miR-3689b-5p | UGUGAUAUCAUGGUUCC | 1479 | UCCCAGGAACCAUGAUAU | 1480 |
| | UGGGA | | CACA | |
| miR-3689c | CUGGGAGGUGUGAUAUU | 1481 | ACCACAAUAUCACACCUC | 1482 |
| | GUGGU | | CCAG | |
| miR-3689d | GGGAGGUGUGAUCUCAC | 1483 | CGAGUGUGAGAUCACACC | 1484 |
| | ACUCG | | UCCC | |
| miR-3689e | UGUGAUAUCAUGGUUCC | 1485 | UCCCAGGAACCAUGAUAU | 1486 |
| | UGGGA | | CACA | |
| miR-3689f | UGUGAUAUCGUGCUUCC | 1487 | UCCCAGGAAGCACGAUAU | 1488 |
| | UGGGA | | CACA | |
| miR-369-3p | AAUAAUACAUGGUUGAU | 1489 | AAAGAUCAACCAUGUAUU | 1490 |
| | CUUU | | AUU | |
| miR-369-5p | AGAUCGACCGUGUUAUA | 1491 | GCGAAUAUAACACGGUCG | 1492 |
| | UUCGC | | AUCU | |
| miR-3690 | ACCUGGACCCAGCGUAG | 1493 | CUUUGUCUACGCUGGGUC | 1494 |
| | ACAAAG | | CAGGU | |
| miR-3691-3p | ACCAAGUCUGCGUCAUC | 1495 | GAGAGGAUGACGCAGACU | 1496 |
| | CUCUC | | UGGU | |
| miR-3691-5p | AGUGGAUGAUGGAGACU | 1497 | GUACCGAGUCUCCAUCAU | 1498 |
| | CGGUAC | | CCACU | |
| miR-3692-3p | GUUCCACACUGACACUG | 1499 | ACUUCUGCAGUGUCAGUG | 1500 |
| | CAGAAGU | | UGGAAC | |
| miR-3692-5p | CCUGCUGGUCAGGAGUG | 1501 | CAGUAUCCACUCCUGACC | 1502 |
| | GAUACUG | | AGCAGG | |
| miR-370 | GCCUGCUGGGGUGGAAC | 1503 | ACCAGGUUCCACCCCAGC | 1504 |
| | CUGGU | | AGGC | |
| miR-3713 | GGUAUCCGUUUGGGGAU | 1505 | ACCAUCCCCAAACGGAUA | 1506 |
| | GGU | | CC | |
| miR-3714 | GAAGGCAGCAGUGCUCC | 1507 | ACAGGGGAGCACUGCUGC | 1508 |
| | CCUGU | | CUUC | |
| miR-371a-3p | AAGUGCCGCCAUCUUUU | 1509 | ACACUCAAAAGAUGGCGG | 1510 |
| | GAGUGU | | CACUU | |
| miR-371a-5p | ACUCAAACUGUGGGGGC | 1511 | AGUGCCCCCACAGUUUGA | 1512 |
| | ACU | | GU | |
| miR-371b-3p | AAGUGCCCCCACAGUUU | 1513 | GCACUCAAACUGUGGGGG | 1514 |
| | GAGUGC | | CACUU | |
| miR-371b-5p | ACUCAAAAGAUGGCGGC | 1515 | AAAGUGCCGCCAUCUUUU | 1516 |
| | ACUUU | | GAGU | |
| miR-372 | AAAGUGCUGCGACAUUU | 1517 | ACGCUCAAAUGUCGCAGC | 1518 |
| | GAGCGU | | ACUUU | |
| miR-373-3p | GAAGUGCUUCGAUUUUG | 1519 | ACACCCCAAAAUCGAAGC | 1520 |
| | GGGUGU | | ACUUC | |
| miR-373-5p | ACUCAAAAUGGGGGCGC | 1521 | GGAAAGCGCCCCCAUUUU | 1522 |
| | UUUCC | | GAGU | |
| miR-374a-3p | CUUAUCAGAUUGUAUUG | 1523 | AAUUACAAUACAAUCUGA | 1524 |
| | UAAUU | | UAAG | |
| miR-374a-5p | UUAUAAUACAACCUGAU | 1525 | CACUUAUCAGGUUGUAUU | 1526 |
| | AAGUG | | AUAA | |
| miR-374b-3p | CUUAGCAGGUUGUAUUA | 1527 | AAUGAUAAUACAACCUGC | 1528 |
| | UCAUU | | UAAG | |
| miR-374b-5p | AUAUAAUACAACCUGCU | 1529 | CACUUAGCAGGUUGUAUU | 1530 |
| | AAGUG | | AUAU | |
| miR-374c-3p | CACUUAGCAGGUUGUAU | 1531 | AUAUAAUACAACCUGCUA | 1532 |
| | UAUAU | | AGUG | |
| miR-374c-5p | AUAAUACAACCUGCUAA | 1533 | AGCACUUAGCAGGUUGUA | 1534 |
| | GUGCU | | UUAU | |
| miR-375 | UUUGUUCGUUCGGCUCG | 1535 | UCACGCGAGCCGAACGAA | 1536 |
| | CGUGA | | CAAA | |
| miR-376a-3p | AUCAUAGAGGAAAAUCC | 1537 | ACGUGGAUUUUCCUCUAU | 1538 |
| | ACGU | | GAU | |
| miR-376a-5p | GUAGAUUCUCCUUCUAU | 1539 | UACUCAUAGAAGGAGAA | 1540 |
| | GAGUA | | UCUAC | |
| miR-376b | AUCAUAGAGGAAAAUCC | 1541 | AACAUGGAUUUUCCUCUA | 1542 |
| | AUGUU | | UGAU | |
| miR-376c | AACAUAGAGGAAAUUCC | 1543 | ACGUGGAAUUUCCUCUAU | 1544 |
| | ACGU | | GUU | |
| miR-377-3p | AUCACACAAAGGCAACU | 1545 | ACAAAAGUUGCCUUUGUG | 1546 |
| | UUUGU | | UGAU | |
| miR-377-5p | AGAGGUUGCCCUUGGUG | 1547 | GAAUUCACCAAGGGCAAC | 1548 |
| | AAUUC | | CUCU | |
| miR-378a-3p | ACUGGACUUGGAGUCAG | 1549 | CCUUCUGACUCCAAGUCC | 1550 |
| | AAGG | | AGU | |
| miR-378a-5p | CUCCUGACUCCAGGUCC | 1551 | ACACAGGACCUGGAGUCA | 1552 |
| | UGUGU | | GGAG | |
| miR-378b | ACUGGACUUGGAGGCAG | 1553 | UUCUGCCUCCAAGUCCAG | 1554 |
| | AA | | U | |
| miR-378c | ACUGGACUUGGAGUCAG | 1555 | CCACUCUUCUGACUCCAA | 1556 |
| | AAGAGUGG | | GUCCAGU | |
| miR-378d | ACUGGACUUGGAGUCAG | 1557 | UUUCUGACUCCAAGUCCA | 1558 |
| | AAA | | GU | |
| miR-378e | ACUGGACUUGGAGUCAG | 1559 | UCCUGACUCCAAGUCCAG | 1560 |
| | GA | | U | |
| miR-378f | ACUGGACUUGGAGCCAG | 1561 | CUUCUGGCUCCAAGUCCA | 1562 |
| | AAG | | GU | |
| miR-378g | ACUGGGCUUGGAGUCAG | 1563 | CUUCUGACUCCAAGCCCA | 1564 |
| | AAG | | GU | |
| miR-378h | ACUGGACUUGGUGUCAG | 1565 | CCAUCUGACACCAAGUCC | 1566 |
| | AUGG | | AGU | |
| miR-378i | ACUGGACUAGGAGUCAG | 1567 | CCUUCUGACUCCUAGUCC | 1568 |
| | AAGG | | AGU | |
| miR-379-3p | UAUGUAACAUGGUCCAC | 1569 | AGUUAGUGGACCAUGUU | 1570 |
| | UAACU | | ACAUA | |
| miR-379-5p | UGGUAGACUAUGGAACG | 1571 | CCUACGUUCCAUAGUCUA | 1572 |
| | UAGG | | CCA | |
| miR-380-3p | UAUGUAAUAUGGUCCAC | 1573 | AAGAUGUGGACCAUAUU | 1574 |
| | AUCUU | | ACAUA | |
| miR-380-5p | UGGUUGACCAUAGAACA | 1575 | GCGCAUGUUCUAUGGUCA | 1576 |
| | UGCGC | | ACCA | |
| miR-381 | UAUACAAGGGCAAGCUC | 1577 | ACAGAGAGCUUGCCCUUG | 1578 |
| | UCUGU | | UAUA | |
| miR-382-3p | AAUCAUUCACGGACAAC | 1579 | AAGUGUUGUCCGUGAAU | 1580 |
| | ACUU | | GAUU | |
| miR-382-5p | GAAGUUGUUCGUGGUGG | 1581 | CGAAUCCACCACGAACAA | 1582 |
| | AUUCG | | CUUC | |
| miR-383 | AGAUCAGAAGGUGAUUG | 1583 | AGCCACAAUCACCUUCUG | 1584 |
| | UGGCU | | AUCU | |
| miR-384 | AUUCCUAGAAAUUGUUC | 1585 | UAUGAACAAUUUCUAGG | 1586 |
| | AUA | | AAU | |
| miR-3907 | AGGUGCUCCAGGCUGGC | 1587 | UGUGAGCCAGCCUGGAGC | 1588 |
| | UCACA | | ACCU | |
| miR-3908 | GAGCAAUGUAGGUAGAC | 1589 | AAACAGUCUACCUACAUU | 1590 |
| | UGUUU | | GCUC | |
| miR-3909 | UGUCCUCUAGGGCCUGC | 1591 | AGACUGCAGGCCCUAGAG | 1592 |
| | AGUCU | | GACA | |
| miR-3910 | AAAGGCAUAAAACCAAG | 1593 | UGUCUUGGUUUUAUGCCU | 1594 |
| | ACA | | UU | |
| miR-3911 | UGUGUGGAUCCUGGAGG | 1595 | UGCCUCCUCCAGGAUCCA | 1596 |
| | AGGCA | | CACA | |
| miR-3912 | UAACGCAUAAUAUGGAC | 1597 | ACAUGUCCAUAUUAUGCG | 1598 |
| | AUGU | | UUA | |
| miR-3913-3p | AGACAUCAAGAUCAGUC | 1599 | UUUGGGACUGAUCUUGA | 1600 |
| | CCAAA | | UGUCU | |
| miR-3913-5p | UUUGGGACUGAUCUUGA | 1601 | AGACAUCAAGAUCAGUCC | 1602 |
| | UGUCU | | CAAA | |
| miR-3914 | AAGGAACCAGAAAAUGA | 1603 | ACUUCUCAUUUUCUGGUU | 1604 |
| | GAAGU | | CCUU | |
| miR-3915 | UUGAGGAAAAGAUGGUC | 1605 | AAUAAGACCAUCUUUUCC | 1606 |
| | UUAUU | | UCAA | |
| miR-3916 | AAGAGGAAGAAAUGGCU | 1607 | CUGAGAACCAGCCAUUUC | 1608 |
| | GGUUCUCAG | | UUCCUCUU | |
| miR-3917 | GCUCGGACUGAGCAGGU | 1609 | CCCACCUGCUCAGUCCGA | 1610 |
| | GGG | | GC | |
| miR-3918 | ACAGGGCCGCAGAUGGA | 1611 | AGUCUCCAUCUGCGGCCC | 1612 |
| | GACU | | UGU | |
| miR-3919 | GCAGAGAACAAAGGACU | 1613 | ACUGAGUCCUUUGUUCUC | 1614 |
| | CAGU | | UGC | |
| miR-3920 | ACUGAUUAUCUUAACUC | 1615 | UCAGAGAGUUAAGAUAA | 1616 |
| | UCUGA | | UCAGU | |
| miR-3921 | UCUCUGAGUACCAUAUG | 1617 | ACAAGGCAUAUGGUACUC | 1618 |
| | CCUUGU | | AGAGA | |
| miR-3922-3p | UCUGGCCUUGACUUGAC | 1619 | AAAGAGUCAAGUCAAGGC | 1620 |
| | UCUUU | | CAGA | |
| miR-3922-5p | UCAAGGCCAGAGGUCCC | 1621 | UGCUGUGGGACCUCUGGC | 1622 |
| | ACAGCA | | CUUGA | |
| miR-3923 | AACUAGUAAUGUUGGAU | 1623 | CCCUAAUCCAACAUUACU | 1624 |
| | UAGGG | | AGUU | |
| miR-3924 | AUAUGUAUAUGUGACUG | 1625 | AGUAGCAGUCACAUAUAC | 1626 |
| | CUACU | | AUAU | |
| miR-3925-3p | ACUCCAGUUUUAGUUCU | 1627 | CAAGAGAACUAAAACUGG | 1628 |
| | CUUG | | AGU | |
| miR-3925-5p | AAGAGAACUGAAAGUGG | 1629 | AGGCUCCACUUUCAGUUC | 1630 |
| | AGCCU | | UCUU | |
| miR-3926 | UGGCCAAAAAGCAGGCA | 1631 | UCUCUGCCUGCUUUUUGG | 1632 |
| | GAGA | | CCA | |
| miR-3927 | CAGGUAGAUAUUUGAUA | 1633 | AUGCCUAUCAAAUAUCUA | 1634 |
| | GGCAU | | CCUG | |
| miR-3928 | GGAGGAACCUUGGAGCU | 1635 | GCCGAAGCUCCAAGGUUC | 1636 |
| | UCGGC | | CUCC | |
| miR-3929 | GAGGCUGAUGUGAGUAG | 1637 | AGUGGUCUACUCACAUCA | 1638 |
| | ACCACU | | GCCUC | |
| miR-3934 | UCAGGUGUGGAAACUGA | 1639 | CUGCCUCAGUUUCCACAC | 1640 |
| | GGCAG | | CUGA | |
| miR-3935 | UGUAGAUACGAGCACCA | 1641 | GUGGCUGGUGCUCGUAUC | 1642 |
| | GCCAC | | UACA | |
| miR-3936 | UAAGGGGUGUAUGGCAG | 1643 | UGCAUCUGCCAUACACCC | 1644 |
| | AUGCA | | CUUA | |
| miR-3937 | ACAGGCGGCUGUAGCAA | 1645 | CCCCCAUUGCUACAGCCG | 1646 |
| | UGGGGG | | CCUGU | |
| miR-3938 | AAUUCCCUUGUAGAUAA | 1647 | CCGGGUUAUCUACAAGGG | 1648 |
| | CCCGG | | AAUU | |
| miR-3939 | UACGCGCAGACCACAGG | 1649 | GACAUCCUGUGGUCUGCG | 1650 |
| | AUGUC | | CGUA | |
| miR-3940-3p | CAGCCCGGAUCCCAGCC | 1651 | AAGUGGGCUGGGAUCCGG | 1652 |
| | CACUU | | GCUG | |
| miR-3940-5p | GUGGGUUGGGGCGGGCU | 1653 | CAGAGCCCGCCCCAACCC | 1654 |
| | CUG | | AC | |
| miR-3941 | UUACACACAACUGAGGA | 1655 | UAUGAUCCUCAGUUGUGU | 1656 |
| | UCAUA | | GUAA | |
| miR-3942-3p | UUUCAGAUAACAGUAUU | 1657 | AUGUAAUACUGUUAUCU | 1658 |
| | ACAU | | GAAA | |
| miR-3942-5p | AAGCAAUACUGUUACCU | 1659 | AUUUCAGGUAACAGUAU | 1660 |
| | GAAAU | | UGCUU | |
| miR-3943 | UAGCCCCCAGGCUUCAC | 1661 | CGCCAAGUGAAGCCUGGG | 1662 |
| | UUGGCG | | GGCUA | |
| miR-3944-3p | UUCGGGCUGGCCUGCUG | 1663 | CCGGAGCAGCAGGCCAGC | 1664 |
| | CUCCGG | | CCGAA | |
| miR-3944-5p | UGUGCAGCAGGCCAACC | 1665 | UCUCGGUUGGCCUGCUGC | 1666 |
| | GAGA | | ACA | |
| miR-3945 | AGGGCAUAGGAGAGGGU | 1667 | AUAUCAACCCUCUCCUAU | 1668 |
| | UGAUAU | | GCCCU | |
| miR-3960 | GGCGGCGGCGGAGGCGG | 1669 | CCCCCGCCTCCGCCGCCGC | 1670 |
| | GGG | | C | |
| miR-3972 | CUGCCAGCCCCGUUCCA | 1671 | UGCCCUGGAACGGGGCUG | 1672 |
| | GGGCA | | GCAG | |
| miR-3973 | ACAAAGUACAGCAUUAG | 1673 | CUAAGGCUAAUGCUGUAC | 1674 |
| | CCUUAG | | UUUGU | |
| miR-3974 | AAAGGUCAUUGUAAGGU | 1675 | GCAUUAACCUUACAAUGA | 1676 |
| | UAAUGC | | CCUUU | |
| miR-3975 | UGAGGCUAAUGCACUAC | 1677 | GUGAAGUAGUGCAUUAG | 1678 |
| | UUCAC | | CCUCA | |
| miR-3976 | UAUAGAGAGCAGGAAGA | 1679 | ACAUUAAUCUUCCUGCUC | 1680 |
| | UUAAUGU | | UCUAUA | |
| miR-3977 | GUGCUUCAUCGUAAUUA | 1681 | UAAGGUUAAUUACGAUG | 1682 |
| | ACCUUA | | AAGCAC | |
| miR-3978 | GUGGAAAGCAUGCAUCC | 1683 | ACACCCUGGAUGCAUGCU | 1684 |
| | AGGGUGU | | UUCCAC | |
| miR-409-3p | GAAUGUUGCUCGGUGAA | 1685 | AGGGGUUCACCGAGCAAC | 1686 |
| | CCCCU | | AUUC | |
| miR-409-5p | AGGUUACCCGAGCAACU | 1687 | AUGCAAAGUUGCUCGGGU | 1688 |
| | UUGCAU | | AACCU | |
| miR-410 | AAUAUAACACAGAUGGC | 1689 | ACAGGCCAUCUGUGUUAU | 1690 |
| | CUGU | | AUU | |
| miR-411-3p | UAUGUAACACGGUCCAC | 1691 | GGUUAGUGGACCGUGUU | 1692 |
| | UAACC | | ACAUA | |
| miR-411-5p | UAGUAGACCGUAUAGCG | 1693 | CGUACGCUAUACGGUCUA | 1694 |
| | UACG | | CUA | |
| miR-412 | ACUUCACCUGGUCCACU | 1695 | ACGGCUAGUGGACCAGGU | 1696 |
| | AGCCGU | | GAAGU | |
| miR-421 | AUCAACAGACAUUAAUU | 1697 | GCGCCCAAUUAAUGUCUG | 1698 |
| | GGGCGC | | UUGAU | |
| miR-422a | ACUGGACUUAGGGUCAG | 1699 | GCCUUCUGACCCUAAGUC | 1700 |
| | AAGGC | | CAGU | |
| miR-423-3p | AGCUCGGUCUGAGGCCC | 1701 | ACUGAGGGGCCUCAGACC | 1702 |
| | CUCAGU | | GAGCU | |
| miR-423-5p | UGAGGGGCAGAGAGCGA | 1703 | AAAGUCUCGCUCUCUGCC | 1704 |
| | GACUUU | | CCUCA | |
| miR-424-3p | CAAAACGUGAGGCGCUG | 1705 | AUAGCAGCGCCUCACGUU | 1706 |
| | CUAU | | UUG | |
| miR-424-5p | CAGCAGCAAUUCAUGUU | 1707 | UUCAAAACAUGAAUUGCU | 1708 |
| | UUGAA | | GCUG | |
| miR-425-3p | AUCGGGAAUGUCGUGUC | 1709 | GGGCGGACACGACAUUCC | 1710 |
| | CGCCC | | CGAU | |
| miR-425-5p | AAUGACACGAUCACUCC | 1711 | UCAACGGGAGUGAUCGUG | 1712 |
| | CGUUGA | | UCAUU | |
| miR-4251 | CCUGAGAAAAGGGCCAA | 1713 | UUGGCCCUUUUCUCAGG | 1714 |
| miR-4252 | GGCCACUGAGUCAGCAC | 1715 | UGGUGCUGACUCAGUGGC | |
| | CA | | C | 1716 |
| miR-4253 | AGGGCAUGUCCAGGGGG | 1717 | ACCCCCUGGACAUGCCCU | 1718 |
| | U | | | |
| miR-4254 | GCCUGGAGCUACUCCAC | 1719 | GAGAUGGUGGAGUAGCU | 1720 |
| | CAUCUC | | CCAGGC | |
| miR-4255 | CAGUGUUCAGAGAUGGA | 1721 | UCCAUCUCUGAACACUG | 1722 |
| miR-4256 | AUCUGACCUGAUGAAGG | 1723 | ACCUUCAUCAGGUCAGAU | 1724 |
| | U | | | |
| miR-4257 | CCAGAGGUGGGGACUGA | 1725 | CUCAGUCCCCACCUCUGG | 1726 |
| | G | | | |
| miR-4258 | CCCCGCCACCGCCUUGG | 1727 | CCAAGGCGGUGGCGGGG | 1728 |
| miR-4259 | CAGUUGGGUCUAGGGGU | 1729 | UCCUGACCCCUAGACCCA | 1730 |
| | CAGGA | | ACUG | |
| miR-4260 | CUUGGGGCAUGGAGUCC | 1731 | UGGGACUCCAUGCCCCAA | 1732 |
| | CA | | G | |
| miR-4261 | AGGAAACAGGGACCCA | 1733 | TGGGTCCCTGTTTCCT | 1734 |
| miR-4262 | GACAUUCAGACUACCUG | 1735 | CAGGUAGUCUGAAUGUC | 1736 |
| miR-4263 | AUUCUAAGUGCCUUGGC | 1737 | GGCCAAGGCACUUAGAAU | 1738 |
| | C | | | |
| miR-4264 | ACUCAGUCAUGGUCAUU | 1739 | AAUGACCAUGACUGAGU | 1740 |
| miR-4265 | CUGUGGGCUCAGCUCUG | 1741 | CCCAGAGCUGAGCCCACA | 1742 |
| | GG | | G | |
| miR-4266 | CUAGGAGGCCUUGGCC | 1743 | GGCCAAGGCCUCCUAG | 1744 |
| miR-4267 | UCCAGCUCGGUGGCAC | 1745 | GUGCCACCGAGCUGGA | 1746 |
| miR-4268 | GGCUCCUCCUCUCAGGA | 1747 | CACAUCCUGAGAGGAGGA | 1748 |
| | UGUG | | GCC | |
| miR-4269 | GCAGGCACAGACAGCCC | 1749 | GCCAGGGCUGUCUGUGCC | 1750 |
| | UGGC | | UGC | |
| miR-4270 | UCAGGGAGUCAGGGGAG | 1751 | GCCCUCCCCUGACUCCCU | 1752 |
| | GGC | | GA | |
| miR-4271 | GGGGGAAGAAAAGGUGG | 1753 | CCCCACCUUUUCUUCCCC | 1754 |
| | GG | | C | |
| miR-4272 | CAUUCAACUAGUGAUUG | 1755 | ACAAUCACUAGUUGAAUG | 1756 |
| | U | | | |
| miR-4273 | GUGUUCUCUGAUGGACA | 1757 | CUGUCCAUCAGAGAACAC | 1758 |
| | G | | | |
| miR-4274 | CAGCAGUCCCUCCCCCU | 1759 | CAGGGGGAGGGACUGCUG | 1760 |
| | G | | | |
| miR-4275 | CCAAUUACCACUUCUUU | 1761 | AAAGAAGUGGUAAUUGG | 1762 |
| miR-4276 | CUCAGUGACUCAUGUGC | 1763 | GCACAUGAGUCACUGAG | 1764 |
| miR-4277 | GCAGUUCUGAGCACAGU | 1765 | GUGUACUGUGCUCAGAAC | 1766 |
| | ACAC | | UGC | |
| miR-4278 | CUAGGGGGUUUGCCCUU | 1767 | CAAGGGCAAACCCCCUAG | 1768 |
| | G | | | |
| miR-4279 | CUCUCCUCCCGGCUUC | 1769 | GAAGCCGGGAGGAGAG | 1770 |
| miR-4280 | GAGUGUAGUUCUGAGCA | 1771 | GCUCUGCUCAGAACUACA | 1772 |
| | GAGC | | CUC | |
| miR-4281 | GGGUCCCGGGGAGGGGG | 1773 | CCCCCCUCCCCGGGACCC | 1774 |
| | G | | | |
| miR-4282 | UAAAAUUUGCAUCCAGG | 1775 | UCCUGGAUGCAAAUUUUA | 1776 |
| | A | | | |
| miR-4283 | UGGGGCUCAGCGAGUUU | 1777 | AAACUCGCUGAGCCCCA | 1778 |
| miR-4284 | GGGCUCACAUCACCCCA | 1779 | AUGGGGUGAUGUGAGCCC | 1780 |
| | U | | | |
| miR-4285 | GCGGCGAGUCCGACUCA | 1781 | AUGAGUCGGACUCGCCGC | 1782 |
| | U | | | |
| miR-4286 | ACCCCACUCCUGGUACC | 1783 | GGUACCAGGAGUGGGGU | 1784 |
| miR-4287 | UCUCCCUUGAGGGCACU | 1785 | AAAGUGCCCUCAAGGGAG | 1786 |
| | UU | | A | |
| miR-4288 | UUGUCUGCUGAGUUUCC | 1787 | GGAAACUCAGCAGACAA | 1788 |
| miR-4289 | GCAUUGUGCAGGGCUAU | 1789 | UGAUAGCCCUGCACAAUG | 1790 |
| | CA | | C | |
| miR-429 | UAAUACUGUCUGGUAAA | 1791 | ACGGUUUUACCAGACAGU | 1792 |
| | ACCGU | | AUUA | |
| miR-4290 | UGCCCUCCUUUCUUCCC | 1793 | GAGGGAAGAAAGGAGGG | 1794 |
| | UC | | CA | |
| miR-4291 | UUCAGCAGGAACAGCU | 1795 | AGCUGUUCCUGCUGAA | 1796 |
| miR-4292 | CCCCUGGGCCGGCCUUG | 1797 | CCAAGGCCGGCCCAGGGG | 1798 |
| | G | | | |
| miR-4293 | CAGCCUGACAGGAACAG | 1799 | CUGUUCCUGUCAGGCUG | 1800 |
| miR-4294 | GGGAGUCUACAGCAGGG | 1801 | CCCUGCUGUAGACUCCC | 1802 |
| miR-4295 | CAGUGCAAUGUUUUCCU | 1803 | AAGGAAAACAUUGCACUG | 1804 |
| | U | | | |
| miR-4296 | AUGUGGGCUCAGGCUCA | 1805 | UGAGCCUGAGCCCACAU | 1806 |
| miR-4297 | UGCCUUCCUGUCUGUG | 1807 | CACAGACAGGAAGGCA | 1808 |
| miR-4298 | CUGGGACAGGAGGAGGA | 1809 | CUGCCUCCUCCUCCUGUC | 1810 |
| | GGCAG | | CCAG | |
| miR-4299 | GCUGGUGACAUGAGAGG | 1811 | GCCUCUCAUGUCACCAGC | 1812 |
| | C | | | |
| miR-4300 | UGGGAGCUGGACUACUU | 1813 | GAAGUAGUCCAGCUCCCA | 1814 |
| | C | | | |
| miR-4301 | UCCCACUACUUCACUUG | 1815 | UCACAAGUGAAGUAGUG | 1816 |
| | UGA | | GGA | |
| miR-4302 | CCAGUGUGGCUCAGCGA | 1817 | CUCGCUGAGCCACACUGG | 1818 |
| | G | | | |
| miR-4303 | UUCUGAGCUGAGGACAG | 1819 | CUGUCCUCAGCUCAGAA | 1820 |
| miR-4304 | CCGGCAUGUCCAGGGCA | 1821 | UGCCCUGGACAUGCCGG | 1822 |
| miR-4305 | CCUAGACACCUCCAGUU | 1823 | GAACUGGAGGUGUCUAG | 1824 |
| | C | | G | |
| miR-4306 | UGGAGAGAAAGGCAGUA | 1825 | UACUGCCUUUCUCUCCA | 1826 |
| miR-4307 | AAUGUUUUUUCCUGUUU | 1827 | GGAAACAGGAAAAAACA | 1828 |
| | CC | | UU | |
| miR-4308 | UCCCUGGAGUUUCUUCU | 1829 | AAGAAGAAACUCCAGGGA | 1830 |
| | U | | | |
| miR-4309 | CUGGAGUCUAGGAUUCC | 1831 | UGGAAUCCUAGACUCCAG | 1832 |
| | A | | | |
| miR-431-3p | CAGGUCGUCUUGCAGGG | 1833 | AGAAGCCCUGCAAGACGA | 1834 |
| | CUUCU | | CCUG | |
| miR-431-5p | UGUCUUGCAGGCCGUCA | 1835 | UGCAUGACGGCCUGCAAG | 1836 |
| | UGCA | | ACA | |
| miR-4310 | GCAGCAUUCAUGUCCC | 1837 | GGGACAUGAAUGCUGC | 1838 |
| miR-4311 | GAAAGAGAGCUGAGUGU | 1839 | CACACUCAGCUCUCUUUC | 1840 |
| | G | | | |
| miR-4312 | GCCUUGUUCCUGUCCC | 1841 | UGGGGACAGGAACAAGGC | 1842 |
| | CA | | C | |
| miR-4313 | AGCCCCCUGGCCCCAAA | 1843 | GGGUUUGGGGCCAGGGG | 1844 |
| | CCC | | GCU | |
| miR-4314 | CUCUGGGAAAUGGGACA | 1845 | CUGUCCCAUUUCCCAGAG | 1846 |
| | G | | | |
| miR-4315 | CCGCUUUCUGAGCUGGA | 1847 | GUCCAGCUCAGAAAGCGG | 1848 |
| | C | | | |
| miR-4316 | GGUGAGGCUAGCUGGUG | 1849 | CACCAGCUAGCCUCACC | 1850 |
| miR-4317 | ACAUUGCCAGGGAGUUU | 1851 | AAACUCCCUGGCAAUGU | 1852 |
| miR-4318 | CACUGUGGGUACAUGCU | 1853 | AGCAUGUACCCACAGUG | 1854 |
| miR-4319 | UCCCUGAGCAAAGCCAC | 1855 | GUGGCUUUGCUCAGGGA | 1856 |
| miR-432-3p | CUGGAUGGCUCCUCCAU | 1857 | AGACAUGGAGGAGCCAUC | 1858 |
| | GUCU | | CAG | |
| miR-432-5p | UCUUGGAGUAGGUCAUU | 1859 | CCACCCAAUGACCUACUC | 1860 |
| | GGGUGG | | CAAGA | |
| miR-4320 | GGGAUUCUGUAGCUUCC | 1861 | AGGAAGCUACAGAAUCCC | 1862 |
| | U | | | |
| miR-4321 | UUAGCGGUGGACCGCCC | 1863 | CGCAGGGCGGUCCACCGC | 1864 |
| | UGCG | | UAA | |
| miR-4322 | CUGUGGGCUCAGCGCGU | 1865 | CCCCACGCGCUGAGCCCA | 1866 |
| | GGGG | | CAG | |
| miR-4323 | CAGCCCCACAGCCUCAG | 1867 | UCUGAGGCUGUGGGGCUG | 1868 |
| | A | | | |
| miR-4324 | CCCUGAGACCCUAACCU | 1869 | UUAAGGUUAGGGUCUCA | 1870 |
| | UAA | | GGG | |
| miR-4325 | UUGCACUUGUCUCAGUG | 1871 | UCACUGAGACAAGUGCAA | 1872 |
| | A | | | |
| miR-4326 | UGUUCCUCUGUCUCCCA | 1873 | GUCUGGGAGACAGAGGA | 1874 |
| | GAC | | ACA | |
| miR-4327 | GGCUUGCAUGGGGGACU | 1875 | CCAGUCCCCCAUGCAAGC | 1876 |
| | GG | | C | |
| miR-4328 | CCAGUUUUCCCAGGAUU | 1877 | AAUCCUGGGAAAACUGG | 1878 |
| miR-4329 | CCUGAGACCCUAGUUCC | 1879 | GUGGAACUAGGGUCUCAG | 1880 |
| | AC | | G | |
| miR-433 | AUCAUGAUGGGCUCCUC | 1881 | ACACCGAGGAGCCCAUCA | 1882 |
| | GGUGU | | UGAU | |
| miR-4330 | CCUCAGAUCAGAGCCUU | 1883 | GCAAGGCUCUGAUCUGAG | 1884 |
| | GC | | G | |
| miR-4417 | GGUGGGCUUCCCGGAGG | 1885 | CCCUCCGGGAAGCCCACC | 1886 |
| | G | | | |
| miR-4418 | CACUGCAGGACUCAGCA | 1887 | CUGCUGAGUCCUGCAGUG | 1888 |
| | G | | | |
| miR-4419a | UGAGGGAGGAGACUGCA | 1889 | UGCAGUCUCCUCCCUCA | 1890 |
| miR-4419b | GAGGCUGAAGGAAGAUG | 1891 | CCAUCUUCCUUCAGCCUC | 1892 |
| | G | | | |
| miR-4420 | GUCACUGAUGUCUGUAG | 1893 | CUCAGCUACAGACAUCAG | 1894 |
| | CUGAG | | UGAC | |
| miR-4421 | ACCUGUCUGUGGAAAGG | 1895 | UAGCUCCUUUCCACAGAC | 1896 |
| | AGCUA | | AGGU | |
| miR-4422 | AAAAGCAUCAGGAAGUA | 1897 | UGGGUACUUCCUGAUGCU | 1898 |
| | CCCA | | UUU | |
| miR-4423-3p | AUAGGCACCAAAAAGCA | 1899 | UUGUUGCUUUUUGGUGCC | 1900 |
| | ACAA | | UAU | |
| miR-4423-5p | AGUUGCCUUUUUGUUCC | 1901 | GCAUGGGAACAAAAAGGC | 1902 |
| | CAUGC | | AACU | |
| miR-4424 | AGAGUUAACUCAAAAUG | 1903 | UAGUCCAUUUUGAGUUA | 1904 |
| | GACUA | | ACUCU | |
| miR-4425 | UGUUGGGAUUCAGCAGG | 1905 | AUGGUCCUGCUGAAUCCC | 1906 |
| | ACCAU | | AACA | |
| miR-4426 | GAAGAUGGACGUACUUU | 1907 | AAAGUACGUCCAUCUUC | 1908 |
| miR-4427 | UCUGAAUAGAGUCUGAA | 1909 | ACUCUUCAGACUCUAUUC | 1910 |
| | GAGU | | AGA | |
| miR-4428 | CAAGGAGACGGGAACAU | 1911 | GCUCCAUGUUCCCGUCUC | 1912 |
| | GGAGC | | CUUG | |
| miR-4429 | AAAAGCUGGGCUGAGAG | 1913 | CGCCUCUCAGCCCAGCUU | 1914 |
| | GCG | | UU | |
| miR-4430 | AGGCUGGAGUGAGCGGA | 1915 | CUCCGCUCACUCCAGCCU | 1916 |
| | G | | | |
| miR-4431 | GCGACUCUGAAAACUAG | 1917 | ACCUUCUAGUUUUCAGAG | 1918 |
| | AAGGU | | UCGC | |
| miR-4432 | AAAGACUCUGCAAGAUG | 1919 | AGGCAUCUUGCAGAGUCU | 1920 |
| | CCU | | UU | |
| miR-4433-3p | ACAGGAGUGGGGGUGGG | 1921 | AUGUCCCACCCCCACUCC | 1922 |
| | ACAU | | UGU | |
| miR-4433-5p | CGUCCCACCCCCCACUCC | 1923 | ACAGGAGUGGGGGGUGG | 1924 |
| | UGU | | GACG | |
| miR-4434 | AGGAGAAGUAAAGUAGA | 1925 | UUCUACUUUACUUCUCCU | 1926 |
| | A | | | |
| miR-4435 | AUGGCCAGAGCUCACAC | 1927 | CCUCUGUGUGAGCUCUGG | 1928 |
| | AGAGG | | CCAU | |
| miR-4436a | GCAGGACAGGCAGAAGU | 1929 | AUCCACUUCUGCCUGUCC | 1930 |
| | GGAU | | UGC | |
| miR-4436b-3p | CAGGGCAGGAAGAAGUG | 1931 | UUGUCCACUUCUUCCUGC | 1932 |
| | GACAA | | CCUG | |
| miR-4436b-5p | GUCCACUUCUGCCUGCC | 1933 | GGCAGGGCAGGCAGAAGU | 1934 |
| | CUGCC | | GGAC | |
| miR-4437 | UGGGCUCAGGGUACAAA | 1935 | AACCUUUGUACCCUGAGC | 1936 |
| | GGUU | | CCA | |
| miR-4438 | CACAGGCUUAGAAAAGA | 1937 | ACUGUCUUUUCUAAGCCU | 1938 |
| | CAGU | | GUG | |
| miR-4439 | GUGACUGAUACCUUGGA | 1939 | AUGCCUCCAAGGUAUCAG | 1940 |
| | GGCAU | | UCAC | |
| miR-4440 | UGUCGUGGGGCUUGCUG | 1941 | CAAGCCAGCAAGCCCCAC | 1942 |
| | GCUUG | | GACA | |
| miR-4441 | ACAGGGAGGAGAUUGUA | 1943 | UACAAUCUCCUCCCUGU | 1944 |
| miR-4442 | GCCGGACAAGAGGGAGG | 1945 | CCTCCCTCTTGTCCGGC | 1946 |
| miR-4443 | UUGGAGGCGUGGGUUUU | 1947 | AAAACCCACGCCUCCAA | 1948 |
| miR-4444 | CUCGAGUUGGAAGAGGC | 1949 | CGCCUCUUCCAACUCGAG | 1950 |
| | G | | | |
| miR-4445-3p | CACGGCAAAAGAAACAA | 1951 | UGGAUUGUUUCUUUUGCC | 1952 |
| | UCCA | | GUG | |
| miR-4445-5p | AGAUUGUUUCUUUUGCC | 1953 | UGCACGGCAAAAGAAACA | 1954 |
| | GUGCA | | AUCU | |
| miR-4446-3p | CAGGGCUGGCAGUGACA | 1955 | ACCCAUGUCACUGCCAGC | 1956 |
| | UGGGU | | CCUG | |
| miR-4446-5p | AUUUCCCUGCCAUUCCC | 1957 | GCCAAGGGAAUGGCAGGG | 1958 |
| | UUGGC | | AAAU | |
| miR-4447 | GGUGGGGGCUGUUGUUU | 1959 | AAACAACAGCCCCCACC | 1960 |
| miR-4448 | GGCUCCUUGGUCUAGGG | 1961 | UACCCCUAGACCAAGGAG | 1962 |
| | GUA | | CC | |
| miR-4449 | CGUCCCGGGGCUGCGCG | 1963 | UGCCUCGCGCAGCCCCGG | 1964 |
| | AGGCA | | GACG | |
| miR-4450 | UGGGGAUUUGGAGAAGU | 1965 | UCACCACUUCUCCAAAUC | 1966 |
| | GGUGA | | CCCA | |
| miR-4451 | UGGUAGAGCUGAGGACA | 1967 | UGUCCUCAGCUCUACCA | 1968 |
| miR-4452 | UUGAAUUCUUGGCCUUA | 1969 | AUCACUUAAGGCCAAGAA | 1970 |
| | AGUGAU | | UUCAA | |
| miR-4453 | GAGCUUGGUCUGUAGCG | 1971 | AACCGCUACAGACCAAGC | 1972 |
| | GUU | | UC | |
| miR-4454 | GGAUCCGAGUCACGGCA | 1973 | UGGUGCCGUGACUCGGAU | 1974 |
| | CCA | | CC | |
| miR-4455 | AGGGUGUGUGUGUUUUU | 1975 | AAAAACACACACACCCU | 1976 |
| miR-4456 | CCUGGUGGCUUCCUUUU | 1977 | AAAAGGAAGCCACCAGG | 1978 |
| miR-4457 | UCACAAGGUAUUGACUG | 1979 | UACGCCAGUCAAUACCUU | 1980 |
| | GCGUA | | GUGA | |
| miR-4458 | AGAGGUAGGUGUGGAAG | 1981 | UUCUUCCACACCUACCUC | 1982 |
| | AA | | U | |
| miR-4459 | CCAGGAGGCGGAGGAGG | 1983 | CUCCACCUCCUCCGCCUC | 1984 |
| | UGGAG | | CUGG | |
| miR-4460 | AUAGUGGUUGUGAAUUU | 1985 | AAGGUAAAUUCACAACCA | 1986 |
| | ACCUU | | CUAU | |
| miR-4461 | GAUUGAGACUAGUAGGG | 1987 | GCCUAGCCCUACUAGUCU | 1988 |
| | CUAGGC | | CAAUC | |
| miR-4462 | UGACACGGAGGGUGGCU | 1989 | UUCCCAAGCCACCCUCCG | 1990 |
| | UGGGAA | | UGUCA | |
| miR-4463 | GAGACUGGGGUGGGGCC | 1991 | GGCCCCACCCCAGUCUC | 1992 |
| miR-4464 | AAGGUUUGGAUAGAUGC | 1993 | UAUUGCAUCUAUCCAAAC | 1994 |
| | AAUA | | CUU | |
| miR-4465 | CUCAAGUAGUCUGACCA | 1995 | UCCCCUGGUCAGACUACU | 1996 |
| | GGGGA | | UGAG | |
| miR-4466 | GGGUGCGGGCCGGCGGG | 1997 | CCCCGCCGGCCCGCACCC | 1998 |
| | G | | | |
| miR-4467 | UGGCGGCGGUAGUUAUG | 1999 | AAGCCCAUAACUACCGCC | 2000 |
| | GGCUU | | GCCA | |
| miR-4468 | AGAGCAGAAGGAUGAGA | 2001 | AUCUCAUCCUUCUGCUCU | 2002 |
| | U | | | |
| miR-4469 | GCUCCCUCUAGGGUCGC | 2003 | UCCGAGCGACCCUAGAGG | 2004 |
| | UCGGA | | GAGC | |
| miR-4470 | UGGCAAACGUGGAAGCC | 2005 | UCUCGGCUUCCACGUUUG | 2006 |
| | GAGA | | CCA | |
| miR-4471 | UGGGAACUUAGUAGAGG | 2007 | UUAAACCUCUACUAAGUU | 2008 |
| | UUUAA | | CCCA | |
| miR-4472 | GGUGGGGGGUGUUGUUU | 2009 | AAAACAACACCCCCCACC | 2010 |
| | U | | | |
| miR-4473 | CUAGUGCUCUCCGUUAC | 2011 | UACUUGUAACGGAGAGCA | 2012 |
| | AAGUA | | CUAG | |
| miR-4474-3p | UUGUGGCUGGUCAUGAG | 2013 | UUAGCCUCAUGACCAGCC | 2014 |
| | GCUAA | | ACAA | |
| miR-4474-5p | UUAGUCUCAUGAUCAGA | 2015 | UGUGUCUGAUCAUGAGAC | 2016 |
| | CACA | | UAA | |
| miR-4475 | CAAGGGACCAAGCAUUC | 2017 | AUAAUGAAUGCUUGGUCC | 2018 |
| | AUUAU | | CUUG | |
| miR-4476 | CAGGAAGGAUUUAGGGA | 2019 | GCCUGUCCCUAAAUCCUU | 2020 |
| | CAGGC | | CCUG | |
| miR-4477a | CUAUUAAGGACAUUUGU | 2021 | GAAUCACAAAUGUCCUUA | 2022 |
| | GAUUC | | AUAG | |
| miR-4477b | AUUAAGGACAUUUGUGA | 2023 | AUCAAUCACAAAUGUCCU | 2024 |
| | UUGAU | | UAAU | |
| miR-4478 | GAGGCUGAGCUGAGGAG | 2025 | CUCCUCAGCUCAGCCUC | 2026 |
| miR-4479 | CGCGCGGCCGUGCUCGG | 2027 | CUGCUCCGAGCACGGCCG | 2028 |
| | AGCAG | | CGCG | |
| miR-448 | UUGCAUAUGUAGGAUGU | 2029 | AUGGGACAUCCUACAUAU | 2030 |
| | CCCAU | | GCAA | |
| miR-4480 | AGCCAAGUGGAAGUUAC | 2031 | UAAAGUAACUUCCACUUG | 2032 |
| | UUUA | | GCU | |
| miR-4481 | GGAGUGGGCUGGUGGUU | 2033 | AACCACCAGCCCACUCC | 2034 |
| miR-4482-3p | UUUCUAUUUCUCAGUGG | 2035 | GAGCCCCACUGAGAAAUA | 2036 |
| | GGCUC | | GAAA | |
| miR-4482-5p | AACCCAGUGGGCUAUGG | 2037 | CAUUUCCAUAGCCCACUG | 2038 |
| | AAAUG | | GGUU | |
| miR-4483 | GGGGUGGUCUGUUGUUG | 2039 | CAACAACAGACCACCCC | 2040 |
| miR-4484 | AAAAGGCGGGAGAAGCC | 2041 | TGGGGCTTCTCCCGCCTTT | 2042 |
| | CCA | | T | |
| miR-4485 | UAACGGCCGCGGUACCC | 2043 | UUAGGGUACCGCGGCCGU | 2044 |
| | UAA | | UA | |
| miR-4486 | GCUGGGCGAGGCUGGCA | 2045 | UGCCAGCCUCGCCCAGC | 2046 |
| miR-4487 | AGAGCUGGCUGAAGGGC | 2047 | CUGCCCUUCAGCCAGCUC | 2048 |
| | AG | | U | |
| miR-4488 | AGGGGGCGGGCUCCGGC | 2049 | CGCCGGAGCCCGCCCCCU | 2050 |
| | G | | | |
| miR-4489 | UGGGGCUAGUGAUGCAG | 2051 | CGUCCUGCAUCACUAGCC | 2052 |
| | GACG | | CCA | |
| miR-4490 | UCUGGUAAGAGAUUUGG | 2053 | UAUGCCCAAAUCUCUUAC | 2054 |
| | GCAUA | | CAGA | |
| miR-4491 | AAUGUGGACUGGUGUGA | 2055 | UUUGGUCACACCAGUCCA | 2056 |
| | CCAAA | | CAUU | |
| miR-4492 | GGGGCUGGGCGCGCGCC | 2057 | GGCGCGCGCCCAGCCCC | 2058 |
| miR-4493 | AGAAGGCCUUUCCAUCU | 2059 | ACAGAGAUGGAAAGGCCU | 2060 |
| | CUGU | | UCU | |
| miR-4494 | CCAGACUGUGGCUGACC | 2061 | CCUCUGGUCAGCCACAGU | 2062 |
| | AGAGG | | CUGG | |
| miR-4495 | AAUGUAAACAGGCUUUU | 2063 | AGCAAAAAGCCUGUUUAC | 2064 |
| | UGCU | | AUU | |
| miR-4496 | GAGGAAACUGAAGCUGA | 2065 | CCCUCUCAGCUUCAGUUU | 2066 |
| | GAGGG | | CCUC | |
| miR-4497 | CUCCGGGACGGCUGGGC | 2067 | GCCCAGCCGUCCCGGAG | 2068 |
| miR-4498 | UGGGCUGGCAGGGCAAG | 2069 | CAGCACUUGCCCUGCCAG | 2070 |
| | UGCUG | | CCCA | |
| miR-4499 | AAGACUGAGAGGAGGGA | 2071 | UCCCUCCUCUCAGUCUU | 2072 |
| miR-449a | UGGCAGUGUAUUGUUAG | 2073 | ACCAGCUAACAAUACACU | 2074 |
| | CUGGU | | GCCA | |
| miR-449b-3p | CAGCCACAACUACCCUG | 2075 | AGUGGCAGGGUAGUUGU | 2076 |
| | CCACU | | GGCUG | |
| miR-449b-5p | AGGCAGUGUAUUGUUAG | 2077 | GCCAGCUAACAAUACACU | 2078 |
| | CUGGC | | GCCU | |
| miR-449c-3p | UUGCUAGUUGCACUCCU | 2079 | ACAGAGAGGAGUGCAACU | 2080 |
| | CUCUGU | | AGCAA | |
| miR-449c-5p | UAGGCAGUGUAUUGCUA | 2081 | ACAGCCGCUAGCAAUACA | 2082 |
| | GCGGCUGU | | CUGCCUA | |
| miR-4500 | UGAGGUAGUAGUUUCUU | 2083 | AAGAAACUACUACCUCA | 2084 |
| miR-4501 | UAUGUGACCUCGGAUGA | 2085 | UGAUUCAUCCGAGGUCAC | 2086 |
| | AUCA | | AUA | |
| miR-4502 | GCUGAUGAUGAUGGUGC | 2087 | CUUCAGCACCAUCAUCAU | 2088 |
| | UGAAG | | CAGC | |
| miR-4503 | UUUAAGCAGGAAAUAGA | 2089 | UAAAUUCUAUUUCCUGCU | 2090 |
| | AUUUA | | UAAA | |
| miR-4504 | UGUGACAAUAGAGAUGA | 2091 | CAUGUUCAUCUCUAUUGU | 2092 |
| | ACAUG | | CACA | |
| miR-4505 | AGGCUGGGCUGGGACGG | 2093 | UCCGUCCCAGCCCAGCCU | 2094 |
| | A | | | |
| miR-4506 | AAAUGGGUGGUCUGAGG | 2095 | UUGCCUCAGACCACCCAU | 2096 |
| | CAA | | UU | |
| miR-4507 | CUGGGUUGGGCUGGGCU | 2097 | CCCAGCCCAGCCCAACCC | 2098 |
| | GGG | | AG | |
| miR-4508 | GCGGGGCUGGGCGCGCG | 2099 | CGCGCGCCCAGCCCCGC | 2100 |
| miR-4509 | ACUAAAGGAUAUAGAAG | 2101 | AAAACCUUCUAUAUCCUU | 2102 |
| | GUUUU | | UAGU | |
| miR-450a-3p | AUUGGGGACAUUUUGCA | 2103 | AUGAAUGCAAAAUGUCCC | 2104 |
| | UUCAU | | CAAU | |
| miR-450a-5p | UUUUGCGAUGUGUUCCU | 2105 | AUAUUAGGAACACAUCGC | 2106 |
| | AAUAU | | AAAA | |
| miR-450b-3p | UUGGGAUCAUUUUGCAU | 2107 | UAUGGAUGCAAAAUGAU | 2108 |
| | CCAUA | | CCCAA | |
| miR-450b-5p | UUUUGCAAUAUGUUCCU | 2109 | UAUUCAGGAACAUAUUGC | 2110 |
| | GAAUA | | AAAA | |
| miR-4510 | UGAGGGAGUAGGAUGUA | 2111 | AACCAUACAUCCUACUCC | 2112 |
| | UGGUU | | CUCA | |
| miR-4511 | GAAGAACUGUUGCAUUU | 2113 | AGGGCAAAUGCAACAGUU | 2114 |
| | GCCCU | | CUUC | |
| miR-4512 | CAGGGCCUCACUGUAUC | 2115 | UGGGCGAUACAGUGAGGC | 2116 |
| | GCCCA | | CCUG | |
| miR-4513 | AGACUGACGGCUGGAGG | 2117 | AUGGGCCUCCAGCCGUCA | 2118 |
| | CCCAU | | GUCU | |
| miR-4514 | ACAGGCAGGAUUGGGGA | 2119 | UUCCCCAAUCCUGCCUGU | 2120 |
| | A | | | |
| miR-4515 | AGGACUGGACUCCCGGC | 2121 | GGGCUGCCGGGAGUCCAG | 2122 |
| | AGCCC | | UCCU | |
| miR-4516 | GGGAGAAGGGUCGGGGC | 2123 | GCCCCGACCCUUCUCCC | 2124 |
| miR-4517 | AAAUAUGAUGAAACUCA | 2125 | CUCAGCUGUGAGUUUCAU | 2126 |
| | CAGCUGAG | | CAUAUUU | |
| miR-4518 | GCUCAGGGAUGAUAACU | 2127 | UCUCAGCACAGUUAUCAU | 2128 |
| | GUGCUGAGA | | CCCUGAGC | |
| miR-4519 | CAGCAGUGCGCAGGGCU | 2129 | CAGCCCUGCGCACUGCUG | 2130 |
| | G | | | |
| miR-451a | AAACCGUUACCAUUACU | 2131 | AACUCAGUAAUGGUAACG | 2132 |
| | GAGUU | | GUUU | |
| miR-451b | UAGCAAGAGAACCAUUA | 2133 | AAUGGUAAUGGUUCUCU | 2134 |
| | CCAUU | | UGCUA | |
| miR-452-3p | CUCAUCUGCAAAGAAGU | 2135 | CACUUACUUCUUUGCAGA | 2136 |
| | AAGUG | | UGAG | |
| miR-452-5p | AACUGUUUGCAGAGGAA | 2137 | UCAGUUUCCUCUGCAAAC | 2138 |
| | ACUGA | | AGUU | |
| miR-4520a-3p | UUGGACAGAAAACACGC | 2139 | UUCCUGCGUGUUUUCUGU | 2140 |
| | AGGAA | | CCAA | |
| miR-4520a-5p | CCUGCGUGUUUUCUGUC | 2141 | UUGGACAGAAAACACGCA | 2142 |
| | CAA | | GG | |
| miR-4520b-3p | UUUGGACAGAAAACACG | 2143 | ACCUGCGUGUUUUCUGUC | 2144 |
| | CAGGU | | CAAA | |
| miR-4520b-5p | CCUGCGUGUUUUCUGUC | 2145 | UUGGACAGAAAACACGCA | 2146 |
| | CAA | | GG | |
| miR-4521 | GCUAAGGAAGUCCUGUG | 2147 | CUGAGCACAGGACUUCCU | 2148 |
| | CUCAG | | UAGC | |
| miR-4522 | UGACUCUGCCUGUAGGC | 2149 | ACCGGCCUACAGGCAGAG | 2150 |
| | CGGU | | UCA | |
| miR-4523 | GACCGAGAGGGCCUCGG | 2151 | ACAGCCGAGGCCCUCUCG | 2152 |
| | CUGU | | GUC | |
| miR-4524a-3p | UGAGACAGGCUUAUGCU | 2153 | AUAGCAGCAUAAGCCUGU | 2154 |
| | GCUAU | | CUCA | |
| miR-4524a-5p | AUAGCAGCAUGAACCUG | 2155 | UGAGACAGGUUCAUGCUG | 2156 |
| | UCUCA | | CUAU | |
| miR-4524b-3p | GAGACAGGUUCAUGCUG | 2157 | UAGCAGCAUGAACCUGUC | 2158 |
| | CUA | | UC | |
| miR-4524b-5p | AUAGCAGCAUAAGCCUG | 2159 | GAGACAGGCUUAUGCUGC | 2160 |
| | UCUC | | UAU | |
| miR-4525 | GGGGGGAUGUGCAUGCU | 2161 | AACCAGCAUGCACAUCCC | 2162 |
| | GGUU | | CCC | |
| miR-4526 | GCUGACAGCAGGGCUGG | 2163 | AGCGGCCAGCCCUGCUGU | 2164 |
| | CCGCU | | CAGC | |
| miR-4527 | UGGUCUGCAAAGAGAUG | 2165 | ACAGUCAUCUCUUUGCAG | 2166 |
| | ACUGU | | ACCA | |
| miR-4528 | UCAUUAUAUGUAUGAUC | 2167 | GUCCAGAUCAUACAUAUA | 2168 |
| | UGGAC | | AUGA | |
| miR-4529-3p | AUUGGACUGCUGAUGGC | 2169 | ACGGGCCAUCAGCAGUCC | 2170 |
| | CCGU | | AAU | |
| miR-4529-5p | AGGCCAUCAGCAGUCCA | 2171 | UUCAUUGGACUGCUGAUG | 2172 |
| | AUGAA | | GCCU | |
| miR-4530 | CCCAGCAGGACGGGAGC | 2173 | CGCTCCCGTCCTGCTGGG | 2174 |
| | G | | | |
| miR-4531 | AUGGAGAAGGCUUCUGA | 2175 | UCAGAAGCCUUCUCCAU | 2176 |
| miR-4532 | CCCCGGGGAGCCCGGCG | 2177 | CGCCGGGCTCCCCGGGG | 2178 |
| miR-4533 | UGGAAGGAGGUUGCCGG | 2179 | AGCGUCCGGCAACCUCCU | 2180 |
| | ACGCU | | UCCA | |
| miR-4534 | GGAUGGAGGAGGGGUCU | 2181 | AGACCCCUCCUCCAUCC | 2182 |
| miR-4535 | GUGGACCUGGCUGGGAC | 2183 | GUCCCAGCCAGGUCCAC | 2184 |
| miR-4536-3p | UCGUGCAUAUAUCUACC | 2185 | AUGUGGUAGAUAUAUGC | 2186 |
| | ACAU | | ACGA | |
| miR-4536-5p | UGUGGUAGAUAUAUGCA | 2187 | AUCGUGCAUAUAUCUACC | 2188 |
| | CGAU | | ACA | |
| miR-4537 | UGAGCCGAGCUGAGCUU | 2189 | CAGCUAAGCUCAGCUCGG | 2190 |
| | AGCUG | | CUCA | |
| miR-4538 | GAGCUUGGAUGAGCUGG | 2191 | UCAGCCCAGCUCAUCCAA | 2192 |
| | GCUGA | | GCUC | |
| miR-4539 | GCUGAACUGGGCUGAGC | 2193 | GCCCAGCUCAGCCCAGUU | 2194 |
| | UGGGC | | CAGC | |
| miR-454-3p | UAGUGCAAUAUUGCUUA | 2195 | ACCCUAUAAGCAAUAUUG | 2196 |
| | UAGGGU | | CACUA | |
| miR-454-5p | ACCCUAUCAAUAUUGUC | 2197 | GCAGAGACAAUAUUGAU | 2198 |
| | UCUGC | | AGGGU | |
| miR-4540 | UUAGUCCUGCCUGUAGG | 2199 | UAAACCUACAGGCAGGAC | 2200 |
| | UUUA | | UAA | |
| miR-455-3p | GCAGUCCAUGGGCAUAU | 2201 | GUGUAUAUGCCCAUGGAC | 2202 |
| | ACAC | | UGC | |
| miR-455-5p | UAUGUGCCUUUGGACUA | 2203 | CGAUGUAGUCCAAAGGCA | 2204 |
| | CAUCG | | CAUA | |
| miR-4632 | UGCCGCCCUCUCGCUGC | 2205 | CUAGAGCAGCGAGAGGGC | 2206 |
| | UCUAG | | GGCA | |
| miR-4633-3p | AGGAGCUAGCCAGGCAU | 2207 | UGCAUAUGCCUGGCUAGC | 2208 |
| | AUGCA | | UCCU | |
| miR-4633-5p | AUAUGCCUGGCUAGCUC | 2209 | GAGGAGCUAGCCAGGCAU | 2210 |
| | CUC | | AU | |
| miR-4634 | CGGCGCGACCGGCCCGG | 2211 | CCCCGGGCCGGTCGCGCC | 2212 |
| | GG | | G | |
| miR-4635 | UCUUGAAGUCAGAACCC | 2213 | UUGCGGGUUCUGACUUCA | 2214 |
| | GCAA | | AGA | |
| miR-4636 | AACUCGUGUUCAAAGCC | 2215 | CUAAAGGCUUUGAACACG | 2216 |
| | UUUAG | | AGUU | |
| miR-4637 | UACUAACUGCAGAUUCA | 2217 | UCACUUGAAUCUGCAGUU | 2218 |
| | AGUGA | | AGUA | |
| miR-4638-3p | CCUGGACACCGCUCAGC | 2219 | CGGCCGGCUGAGCGGUGU | 2220 |
| | CGGCCG | | CCAGG | |
| miR-4638-5p | ACUCGGCUGCGGUGGAC | 2221 | ACUUGUCCACCGCAGCCG | 2222 |
| | AAGU | | AGU | |
| miR-4639-3p | UCACUCUCACCUUGCUU | 2223 | GCAAAGCAAGGUGAGAG | 2224 |
| | UGC | | UGA | |
| miR-4639-5p | UUGCUAAGUAGGCUGAG | 2225 | UCAAUCUCAGCCUACUUA | 2226 |
| | AUUGA | | GCAA | |
| miR-4640-3p | CACCCCCUGUUUCCUGG | 2227 | GUGGGCCAGGAAACAGGG | 2228 |
| | CCCAC | | GGUG | |
| miR-4640-5p | UGGGCCAGGGAGCAGCU | 2229 | CCCACCAGCUGCUCCCUG | 2230 |
| | GGUGGG | | GCCCA | |
| miR-4641 | UGCCCAUGCCAUACUUU | 2231 | UGAGGCAAAAGUAUGGC | 2232 |
| | UGCCUCA | | AUGGGCA | |
| miR-4642 | AUGGCAUCGUCCCCUGG | 2233 | AGCCACCAGGGGACGAUG | 2234 |
| | UGGCU | | CCAU | |
| miR-4643 | GACACAUGACCAUAAAU | 2235 | UUAGCAUUUAUGGUCAU | 2236 |
| | GCUAA | | GUGUC | |
| miR-4644 | UGGAGAGAGAAAAGAGA | 2237 | CUUCUGUCUCUUUUCUCU | 2238 |
| | CAGAAG | | CUCCA | |
| miR-4645-3p | AGACAGUAGUUCUUGCC | 2239 | AACCAGGCAAGAACUACU | 2240 |
| | UGGUU | | GUCU | |
| miR-4645-5p | ACCAGGCAAGAAAUAUU | 2241 | ACAAUAUUUCUUGCCUGG | 2242 |
| | GU | | U | |
| miR-4646-3p | AUUGUCCCUCUCCCUUC | 2243 | CUGGGAAGGGAGAGGGA | 2244 |
| | CCAG | | CAAU | |
| miR-4646-5p | ACUGGGAAGAGGAGCUG | 2245 | UCCCUCAGCUCCUCUUCC | 2246 |
| | AGGGA | | CAGU | |
| miR-4647 | GAAGAUGGUGCUGUGCU | 2247 | UUCCUCAGCACAGCACCA | 2248 |
| | GAGGAA | | UCUUC | |
| miR-4648 | UGUGGGACUGCAAAUGG | 2249 | CUCCCAUUUGCAGUCCCA | 2250 |
| | GAG | | CA | |
| miR-4649-3p | UCUGAGGCCUGCCUCUC | 2251 | UGGGGAGAGGCAGGCCUC | 2252 |
| | CCCA | | AGA | |
| miR-4649-5p | UGGGCGAGGGGUGGGCU | 2253 | CUCUGAGAGCCCACCCCU | 2254 |
| | CUCAGAG | | CGCCCA | |
| miR-4650-3p | AGGUAGAAUGAGGCCUG | 2255 | AUGUCAGGCCUCAUUCUA | 2256 |
| | ACAU | | CCU | |
| miR-4650-5p | UCAGGCCUCUUUCUACC | 2257 | AAGGUAGAAAGAGGCCU | 2258 |
| | UU | | GA | |
| miR-4651 | CGGGGUGGGUGAGGUCG | 2259 | GCCCGACCUCACCCACCC | 2260 |
| | GGC | | CG | |
| miR-4652-3p | GUUCUGUUAACCCAUCC | 2261 | UGAGGGGAUGGGUUAAC | 2262 |
| | CCUCA | | AGAAC | |
| miR-4652-5p | AGGGGACUGGUUAAUAG | 2263 | UAGUUCUAUUAACCAGUC | 2264 |
| | AACUA | | CCCU | |
| miR-4653-3p | UGGAGUUAAGGGUUGCU | 2265 | UCUCCAAGCAACCCUUAA | 2266 |
| | UGGAGA | | CUCCA | |
| miR-4653-5p | UCUCUGAGCAAGGCUUA | 2267 | GGUGUUAAGCCUUGCUCA | 2268 |
| | ACACC | | GAGA | |
| miR-4654 | UGUGGGAUCUGGAGGCA | 2269 | CCAGAUGCCUCCAGAUCC | 2270 |
| | UCUGG | | CACA | |
| miR-4655-3p | ACCCUCGUCAGGUCCCC | 2271 | CCCCGGGGACCUGACGAG | 2272 |
| | GGGG | | GGU | |
| miR-4655-5p | CACCGGGGAUGGCAGAG | 2273 | CGACCCUCUGCCAUCCCC | 2274 |
| | GGUCG | | GGUG | |
| miR-4656 | UGGGCUGAGGGCAGGAG | 2275 | ACAGGCCUCCUGCCCUCA | 2276 |
| | GCCUGU | | GCCCA | |
| miR-4657 | AAUGUGGAAGUGGUCUG | 2277 | AUGCCUCAGACCACUUCC | 2278 |
| | AGGCAU | | ACAUU | |
| miR-4658 | GUGAGUGUGGAUCCUGG | 2279 | AUUCCUCCAGGAUCCACA | 2280 |
| | AGGAAU | | CUCAC | |
| miR-4659a-3p | UUUCUUCUUAGACAUGG | 2281 | CGUUGCCAUGUCUAAGAA | 2282 |
| | CAACG | | GAAA | |
| miR-4659a-5p | CUGCCAUGUCUAAGAAG | 2283 | GUUUUCUUCUUAGACAUG | 2284 |
| | AAAAC | | GCAG | |
| miR-4659b-3p | UUUCUUCUUAGACAUGG | 2285 | AGCUGCCAUGUCUAAGAA | 2286 |
| | CAGCU | | GAAA | |
| miR-4659b-5p | UUGCCAUGUCUAAGAAG | 2287 | UUCUUCUUAGACAUGGCA | 2288 |
| | AA | | A | |
| miR-466 | AUACACAUACACGCAAC | 2289 | AUGUGUGUUGCGUGUAU | 2290 |
| | ACACAU | | GUGUAU | |
| miR-4660 | UGCAGCUCUGGUGGAAA | 2291 | CUCCAUUUUCCACCAGAG | 2292 |
| | AUGGAG | | CUGCA | |
| miR-4661-3p | CAGGAUCCACAGAGCUA | 2293 | UGGACUAGCUCUGUGGAU | 2294 |
| | GUCCA | | CCUG | |
| miR-4661-5p | AACUAGCUCUGUGGAUC | 2295 | GUCAGGAUCCACAGAGCU | 2296 |
| | CUGAC | | AGUU | |
| miR-4662a-3p | AAAGAUAGACAAUUGGC | 2297 | AUUUAGCCAAUUGUCUAU | 2298 |
| | UAAAU | | CUUU | |
| miR-4662a-5p | UUAGCCAAUUGUCCAUC | 2299 | CUAAAGAUGGACAAUUG | 2300 |
| | UUUAG | | GCUAA | |
| miR-4662b | AAAGAUGGACAAUUGGC | 2301 | AUUUAGCCAAUUGUCCAU | 2302 |
| | UAAAU | | CUUU | |
| miR-4663 | AGCUGAGCUCCAUGGAC | 2303 | ACUGCACGUCCAUGGAGC | 2304 |
| | GUGCAGU | | UCAGCU | |
| miR-4664-3p | CUUCCGGUCUGUGAGCC | 2305 | GACGGGGCUCACAGACCG | 2306 |
| | CCGUC | | GAAG | |
| miR-4664-5p | UGGGGUGCCCACUCCGC | 2307 | AACUUGCGGAGUGGGCAC | 2308 |
| | AAGUU | | CCCA | |
| miR-4665-3p | CUCGGCCGCGGCGCGUA | 2309 | GGCGGGGGCUACGCGCCG | 2310 |
| | GCCCCCGCC | | CGGCCGAG | |
| miR-4665-5p | CUGGGGGACGCGUGAGC | 2311 | GCUCGCGCUCACGCGUCC | 2312 |
| | GCGAGC | | CCCAG | |
| miR-4666a-3p | CAUACAAUCUGACAUGU | 2313 | AAAUACAUGUCAGAUUG | 2314 |
| | AUUU | | UAUG | |
| miR-4666a-5p | AUACAUGUCAGAUUGUA | 2315 | GGCAUACAAUCUGACAUG | 2316 |
| | UGCC | | UAU | |
| miR-4666b | UUGCAUGUCAGAUUGUA | 2317 | GGGAAUUACAAUCUGACA | 2318 |
| | AUUCCC | | UGCAA | |
| miR-4667-3p | UCCCUCCUUCUGUCCCC | 2319 | CUGUGGGGACAGAAGGA | 2320 |
| | ACAG | | GGGA | |
| miR-4667-5p | ACUGGGGAGCAGAAGGA | 2321 | GGUUCUCCUUCUGCUCCC | 2322 |
| | GAACC | | CAGU | |
| miR-4668-3p | GAAAAUCCUUUUUGUUU | 2323 | CUGGAAAAACAAAAAGG | 2324 |
| | UUCCAG | | AUUUUC | |
| miR-4668-5p | AGGGAAAAAAAAAAGGA | 2325 | GACAAAUCCUUUUUUUUU | 2326 |
| | UUUGUC | | UCCCU | |
| miR-4669 | UGUGUCCGGGAAGUGGA | 2327 | CCUCCUCCACUUCCCGGA | 2328 |
| | GGAGG | | CACA | |
| miR-4670-3p | UGAAGUUACAUCAUGGU | 2329 | AAGCGACCAUGAUGUAAC | 2330 |
| | CGCUU | | UUCA | |
| miR-4670-5p | AAGCGACCAUGAUGUAA | 2331 | UGAAGUUACAUCAUGGUC | 2332 |
| | CUUCA | | GCUU | |
| miR-4671-3p | UUAGUGCAUAGUCUUUG | 2333 | AGACCAAAGACUAUGCAC | 2334 |
| | GUCU | | UAA | |
| miR-4671-5p | ACCGAAGACUGUGCGCU | 2335 | AGAUUAGCGCACAGUCUU | 2336 |
| | AAUCU | | CGGU | |
| miR-4672 | UUACACAGCUGGACAGA | 2337 | UGCCUCUGUCCAGCUGUG | 2338 |
| | GGCA | | UAA | |
| miR-4673 | UCCAGGCAGGAGCCGGA | 2339 | UCCAGUCCGGCUCCUGCC | 2340 |
| | CUGGA | | UGGA | |
| miR-4674 | CUGGGCUCGGGACGCGC | 2341 | AGCCGCGCGUCCCGAGCC | 2342 |
| | GGCU | | CAG | |
| miR-4675 | GGGGCUGUGAUUGACCA | 2343 | CCUGCUGGUCAAUCACAG | 2344 |
| | GCAGG | | CCCC | |
| miR-4676-3p | CACUGUUUCACCACUGG | 2345 | AAGAGCCAGUGGUGAAAC | 2346 |
| | CUCUU | | AGUG | |
| miR-4676-5p | GAGCCAGUGGUGAGACA | 2347 | UCACUGUCUCACCACUGG | 2348 |
| | GUGA | | CUC | |
| miR-4677-3p | UCUGUGAGACCAAAGAA | 2349 | AGUAGUUCUUUGGUCUCA | 2350 |
| | CUACU | | CAGA | |
| miR-4677-5p | UUGUUCUUUGGUCUUUC | 2351 | UGGCUGAAAGACCAAAGA | 2352 |
| | AGCCA | | ACAA | |
| miR-4678 | AAGGUAUUGUUCAGACU | 2353 | UCAUAAGUCUGAACAAUA | 2354 |
| | UAUGA | | CCUU | |
| miR-4679 | UCUGUGAUAGAGAUUCU | 2355 | AGCAAAGAAUCUCUAUCA | 2356 |
| | UUGCU | | CAGA | |
| miR-4680-3p | UCUGAAUUGUAAGAGUU | 2357 | UAACAACUCUUACAAUUC | 2358 |
| | GUUA | | AGA | |
| miR-4680-5p | AGAACUCUUGCAGUCUU | 2359 | ACAUCUAAGACUGCAAGA | 2360 |
| | AGAUGU | | GUUCU | |
| miR-4681 | AACGGGAAUGCAGGCUG | 2361 | AGAUACAGCCUGCAUUCC | 2362 |
| | UAUCU | | CGUU | |
| miR-4682 | UCUGAGUUCCUGGAGCC | 2363 | AGACCAGGCUCCAGGAAC | 2364 |
| | UGGUCU | | UCAGA | |
| miR-4683 | UGGAGAUCCAGUGCUCG | 2365 | AUCGGGCGAGCACUGGAU | 2366 |
| | CCCGAU | | CUCCA | |
| miR-4684-3p | UGUUGCAAGUCGGUGGA | 2367 | ACGUCUCCACCGACUUGC | 2368 |
| | GACGU | | AACA | |
| miR-4684-5p | CUCUCUACUGACUUGCA | 2369 | UAUGUUGCAAGUCAGUA | 2370 |
| | ACAUA | | GAGAG | |
| miR-4685-3p | UCUCCCUUCCUGCCCUG | 2371 | CUAGCCAGGGCAGGAAGG | 2372 |
| | GCUAG | | GAGA | |
| miR-4685-5p | CCCAGGGCUUGGAGUGG | 2373 | AACCUUGCCCCACUCCAA | 2374 |
| | GGCAAGGUU | | GCCCUGGG | |
| miR-4686 | UAUCUGCUGGGCUUUCU | 2375 | AACACCAGAAAGCCCAGC | 2376 |
| | GGUGUU | | AGAUA | |
| miR-4687-3p | UGGCUGUUGGAGGGGGC | 2377 | GCCUGCCCCCUCCAACAG | 2378 |
| | AGGC | | CCA | |
| miR-4687-5p | CAGCCCUCCUCCCGCACC | 2379 | UUUGGGUGCGGGAGGAG | 2380 |
| | CAAA | | GGCUG | |
| miR-4688 | UAGGGGCAGCAGAGGAC | 2381 | CCCAGGUCCUCUGCUGCC | 2382 |
| | CUGGG | | CCUA | |
| miR-4689 | UUGAGGAGACAUGGUGG | 2383 | GGCCCCCACCAUGUCUCC | 2384 |
| | GGGCC | | UCAA | |
| miR-4690-3p | GCAGCCCAGCUGAGGCC | 2385 | CAGAGGCCUCAGCUGGGC | 2386 |
| | UCUG | | UGC | |
| miR-4690-5p | GAGCAGGCGAGGCUGGG | 2387 | UUCAGCCCAGCCUCGCCU | 2388 |
| | CUGAA | | GCUC | |
| miR-4691-3p | CCAGCCACGGACUGAGA | 2389 | AUGCACUCUCAGUCCGUG | 2390 |
| | GUGCAU | | GCUGG | |
| miR-4691-5p | GUCCUCCAGGCCAUGAG | 2391 | CCGCAGCUCAUGGCCUGG | 2392 |
| | CUGCGG | | AGGAC | |
| miR-4692 | UCAGGCAGUGUGGGUAU | 2393 | AUCUGAUACCCACACUGC | 2394 |
| | CAGAU | | CUGA | |
| miR-4693-3p | UGAGAGUGGAAUUCACA | 2395 | AAAUACUGUGAAUUCCAC | 2396 |
| | GUAUUU | | UCUCA | |
| miR-4693-5p | AUACUGUGAAUUUCACU | 2397 | UGUGACAGUGAAAUUCAC | 2398 |
| | GUCACA | | AGUAU | |
| miR-4694-3p | CAAAUGGACAGGAUAAC | 2399 | AGGUGUUAUCCUGUCCAU | 2400 |
| | ACCU | | UUG | |
| miR-4694-5p | AGGUGUUAUCCUAUCCA | 2401 | GCAAAUGGAUAGGAUAA | 2402 |
| | UUUGC | | CACCU | |
| miR-4695-3p | UGAUCUCACCGCUGCCU | 2403 | GAAGGAGGCAGCGGUGA | 2404 |
| | CCUUC | | GAUCA | |
| miR-4695-5p | CAGGAGGCAGUGGGCGA | 2405 | CCUGCUCGCCCACUGCCU | 2406 |
| | GCAGG | | CCUG | |
| miR-4696 | UGCAAGACGGAUACUGU | 2407 | AGAUGACAGUAUCCGUCU | 2408 |
| | CAUCU | | UGCA | |
| miR-4697-3p | UGUCAGUGACUCCUGCC | 2409 | ACCAAGGGGCAGGAGUCA | 2410 |
| | CCUUGGU | | CUGACA | |
| miR-4697-5p | AGGGGGCGCAGUCACUG | 2411 | CACGUCAGUGACUGCGCC | 2412 |
| | ACGUG | | CCCU | |
| miR-4698 | UCAAAAUGUAGAGGAAG | 2413 | UGGGGUCUUCCUCUACAU | 2414 |
| | ACCCCA | | UUUGA | |
| miR-4699-3p | AAUUUACUCUGCAAUCU | 2415 | GGAGAAGAUUGCAGAGU | 2416 |
| | UCUCC | | AAAUU | |
| miR-4699-5p | AGAAGAUUGCAGAGUAA | 2417 | GGAACUUACUCUGCAAUC | 2418 |
| | GUUCC | | UUCU | |
| miR-4700-3p | CACAGGACUGACUCCUC | 2419 | CACUGGGGUGAGGAGUCA | 2420 |
| | ACCCCAGUG | | GUCCUGUG | |
| miR-4700-5p | UCUGGGGAUGAGGACAG | 2421 | ACACACUGUCCUCAUCCC | 2422 |
| | UGUGU | | CAGA | |
| miR-4701-3p | AUGGGUGAUGGGUGUGG | 2423 | ACACCACACCCAUCACCC | 2424 |
| | UGU | | AU | |
| miR-4701-5p | UUGGCCACCACACCUAC | 2425 | AAGGGGUAGGUGUGGUG | 2426 |
| | CCCUU | | GCCAA | |
| miR-4703-3p | UGUAGUUGUAUUGUAUU | 2427 | GUGGCAAUACAAUACAAC | 2428 |
| | GCCAC | | UACA | |
| miR-4703-5p | UAGCAAUACAGUACAAA | 2429 | ACUAUAUUUGUACUGUA | 2430 |
| | UAUAGU | | UUGCUA | |
| miR-4704-3p | UCAGUCACAUAUCUAGU | 2431 | UAGACACUAGAUAUGUG | 2432 |
| | GUCUA | | ACUGA | |
| miR-4704-5p | GACACUAGGCAUGUGAG | 2433 | AAUCACUCACAUGCCUAG | 2434 |
| | UGAUU | | UGUC | |
| miR-4705 | UCAAUCACUUGGUAAUU | 2435 | ACAGCAAUUACCAAGUGA | 2436 |
| | GCUGU | | UUGA | |
| miR-4706 | AGCGGGGAGGAAGUGGG | 2437 | AAGCAGCGCCCACUUCCU | 2438 |
| | CGCUGCUU | | CCCCGCU | |
| miR-4707-3p | AGCCCGCCCCAGCCGAG | 2439 | AGAACCUCGGCUGGGGCG | 2440 |
| | GUUCU | | GGCU | |
| miR-4707-5p | GCCCCGGCGCGGGCGGG | 2441 | CCAGAACCCGCCCGCGCC | 2442 |
| | UUCUGG | | GGGGC | |
| miR-4708-3p | AGCAAGGCGGCAUCUCU | 2443 | AUCAGAGAGAUGCCGCCU | 2444 |
| | CUGAU | | UGCU | |
| miR-4708-5p | AGAGAUGCCGCCUUGCU | 2445 | AAGGAGCAAGGCGGCAUC | 2446 |
| | CCUU | | UCU | |
| miR-4709-3p | UUGAAGAGGAGGUGCUC | 2447 | GCUACAGAGCACCUCCUC | 2448 |
| | UGUAGC | | UUCAA | |
| miR-4709-5p | ACAACAGUGACUUGCUC | 2449 | UUGGAGAGCAAGUCACUG | 2450 |
| | UCCAA | | UUGU | |
| miR-4710 | GGGUGAGGGCAGGUGGU | 2451 | AACCACCUGCCCUCACCC | 2452 |
| | U | | | |
| miR-4711-3p | CGUGUCUUCUGGCUUGA | 2453 | AUCAAGCCAGAAGACACG | 2454 |
| | U | | | |
| miR-4711-5p | UGCAUCAGGCCAGAAGA | 2455 | CUCAUGUCUUCUGGCCUG | 2456 |
| | CAUGAG | | AUGCA | |
| miR-4712-3p | AAUGAGAGACCUGUACU | 2457 | AUACAGUACAGGUCUCUC | 2458 |
| | GUAU | | AUU | |
| miR-4712-5p | UCCAGUACAGGUCUCUC | 2459 | GAAAUGAGAGACCUGUAC | 2460 |
| | AUUUC | | UGGA | |
| miR-4713-3p | UGGGAUCCAGACAGUGG | 2461 | UUCUCCCACUGUCUGGAU | 2462 |
| | GAGAA | | CCCA | |
| miR-4713-5p | UUCUCCCACUACCAGGC | 2463 | UGGGAGCCUGGUAGUGG | 2464 |
| | UCCCA | | GAGAA | |
| miR-4714-3p | CCAACCUAGGUGGUCAG | 2465 | CAACUCUGACCACCUAGG | 2466 |
| | AGUUG | | UUGG | |
| miR-4714-5p | AACUCUGACCCCUUAGG | 2467 | AUCAACCUAAGGGGUCAG | 2468 |
| | UUGAU | | AGUU | |
| miR-4715-3p | GUGCCACCUUAACUGCA | 2469 | AUUGGCUGCAGUUAAGG | 2470 |
| | GCCAAU | | UGGCAC | |
| miR-4715-5p | AAGUUGGCUGCAGUUAA | 2471 | CCACCUUAACUGCAGCCA | 2472 |
| | GGUGG | | ACUU | |
| miR-4716-3p | AAGGGGGAAGGAAACAU | 2473 | UCUCCAUGUUUCCUUCCC | 2474 |
| | GGAGA | | CCUU | |
| miR-4716-5p | UCCAUGUUUCCUUCCCC | 2475 | AGAAGGGGGAAGGAAAC | 2476 |
| | CUUCU | | AUGGA | |
| miR-4717-3p | ACACAUGGGUGGCUGUG | 2477 | AGGCCACAGCCACCCAUG | 2478 |
| | GCCU | | UGU | |
| miR-4717-5p | UAGGCCACAGCCACCCA | 2479 | ACACAUGGGUGGCUGUGG | 2480 |
| | UGUGU | | CCUA | |
| miR-4718 | AGCUGUACCUGAAACCA | 2481 | UGCUUGGUUUCAGGUACA | 2482 |
| | AGCA | | GCU | |
| miR-4719 | UCACAAAUCUAUAAUAU | 2483 | CCUGCAUAUUAUAGAUUU | 2484 |
| | GCAGG | | GUGA | |
| miR-4720-3p | UGCUUAAGUUGUACCAA | 2485 | AUACUUGGUACAACUUAA | 2486 |
| | GUAU | | GCA | |
| miR-4720-5p | CCUGGCAUAUUUGGUAU | 2487 | AAGUUAUACCAAAUAUGC | 2488 |
| | AACUU | | CAGG | |
| miR-4721 | UGAGGGCUCCAGGUGAC | 2489 | CCACCGUCACCUGGAGCC | 2490 |
| | GGUGG | | CUCA | |
| miR-4722-3p | ACCUGCCAGCACCUCCC | 2491 | CUGCAGGGAGGUGCUGGC | 2492 |
| | UGCAG | | AGGU | |
| miR-4722-5p | GGCAGGAGGGCUGUGCC | 2493 | CAACCUGGCACAGCCCUC | 2494 |
| | AGGUUG | | CUGCC | |
| miR-4723-3p | CCCUCUCUGGCUCCUCCC | 2495 | UUUGGGGAGGAGCCAGA | 2496 |
| | CAAA | | GAGGG | |
| miR-4723-5p | UGGGGGAGCCAUGAGAU | 2497 | UGCUCUUAUCUCAUGGCU | 2498 |
| | AAGAGCA | | CCCCCA | |
| miR-4724-3p | GUACCUUCUGGUUCAGC | 2499 | ACUAGCUGAACCAGAAGG | 2500 |
| | UAGU | | UAC | |
| miR-4724-5p | AACUGAACCAGGAGUGA | 2501 | CGAAGCUCACUCCUGGUU | 2502 |
| | GCUUCG | | CAGUU | |
| miR-4725-3p | UGGGGAAGGCGUCAGUG | 2503 | CCCGACACUGACGCCUUC | 2504 |
| | UCGGG | | CCCA | |
| miR-4725-5p | AGACCCUGCAGCCUUCC | 2505 | GGUGGGAAGGCUGCAGG | 2506 |
| | CACC | | GUCU | |
| miR-4726-3p | ACCCAGGUUCCCUCUGG | 2507 | UGCGGCCAGAGGGAACCU | 2508 |
| | CCGCA | | GGGU | |
| miR-4726-5p | AGGGCCAGAGGAGCCUG | 2509 | CCACUCCAGGCUCCUCUG | 2510 |
| | GAGUGG | | GCCCU | |
| miR-4727-3p | AUAGUGGGAAGCUGGCA | 2511 | GAAUCUGCCAGCUUCCCA | 2512 |
| | GAUUC | | CUAU | |
| miR-4727-5p | AUCUGCCAGCUUCCACA | 2513 | CCACUGUGGAAGCUGGCA | 2514 |
| | GUGG | | GAU | |
| miR-4728-3p | CAUGCUGACCUCCCUCC | 2515 | CUGGGGCAGGAGGGAGG | 2516 |
| | UGCCCCAG | | UCAGCAUG | |
| miR-4728-5p | UGGGAGGGGAGAGGCAG | 2517 | UGCUUGCUGCCUCUCCCC | 2518 |
| | CAAGCA | | UCCCA | |
| miR-4729 | UCAUUUAUCUGUUGGGA | 2519 | UAGCUUCCCAACAGAUAA | 2520 |
| | AGCUA | | AUGA | |
| miR-4730 | CUGGCGGAGCCCAUUCC | 2521 | UGGCAUGGAAUGGGCUCC | 2522 |
| | AUGCCA | | GCCAG | |
| miR-4731-3p | CACACAAGUGGCCCCCA | 2523 | AGUGUUGGGGGCCACUUG | 2524 |
| | ACACU | | UGUG | |
| miR-4731-5p | UGCUGGGGGCCACAUGA | 2525 | CACACUCAUGUGGCCCCC | 2526 |
| | GUGUG | | AGCA | |
| miR-4732-3p | GCCCUGACCUGUCCUGU | 2527 | CAGAACAGGACAGGUCAG | 2528 |
| | UCUG | | GGC | |
| miR-4732-5p | UGUAGAGCAGGGAGCAG | 2529 | AGCUUCCUGCUCCCUGCU | 2530 |
| | GAAGCU | | CUACA | |
| miR-4733-3p | CCACCAGGUCUAGCAUU | 2531 | AUCCCAAUGCUAGACCUG | 2532 |
| | GGGAU | | GUGG | |
| miR-4733-5p | AAUCCCAAUGCUAGACC | 2533 | CACCGGGUCUAGCAUUGG | 2534 |
| | CGGUG | | GAUU | |
| miR-4734 | GCUGCGGGCUGCGGUCA | 2535 | CGCCCUGACCGCAGCCCG | 2536 |
| | GGGCG | | CAGC | |
| miR-4735-3p | AAAGGUGCUCAAAUUAG | 2537 | AUGUCUAAUUUGAGCACC | 2538 |
| | ACAU | | UUU | |
| miR-4735-5p | CCUAAUUUGAACACCUU | 2539 | UACCGAAGGUGUUCAAAU | 2540 |
| | CGGUA | | UAGG | |
| miR-4736 | AGGCAGGUUAUCUGGGC | 2541 | CAGCCCAGAUAACCUGCC | 2542 |
| | UG | | U | |
| miR-4737 | AUGCGAGGAUGCUGACA | 2543 | CACUGUCAGCAUCCUCGC | 2544 |
| | GUG | | AU | |
| miR-4738-3p | UGAAACUGGAGCGCCUG | 2545 | UCCUCCAGGCGCUCCAGU | 2546 |
| | GAGGA | | UUCA | |
| miR-4738-5p | ACCAGCGCGUUUUCAGU | 2547 | AUGAAACUGAAAACGCGC | 2548 |
| | UUCAU | | UGGU | |
| miR-4739 | AAGGGAGGAGGAGCGGA | 2549 | AGGGCCCCUCCGCUCCUC | 2550 |
| | GGGGCCCU | | CUCCCUU | |
| miR-4740-3p | GCCCGAGAGGAUCCGUC | 2551 | GCAGGGACGGAUCCUCUC | 2552 |
| | CCUGC | | GGGC | |
| miR-4740-5p | AGGACUGAUCCUCUCGG | 2553 | CCUGCCCGAGAGGAUCAG | 2554 |
| | GCAGG | | UCCU | |
| miR-4741 | CGGGCUGUCCGGAGGGG | 2555 | AGCCGACCCCUCCGGACA | 2556 |
| | UCGGCU | | GCCCG | |
| miR-4742-3p | UCUGUAUUCUCCUUUGC | 2557 | CUGCAGGCAAAGGAGAAU | 2558 |
| | CUGCAG | | ACAGA | |
| miR-4742-5p | UCAGGCAAAGGGAUAUU | 2559 | UCUGUAAAUAUCCCUUUG | 2560 |
| | UACAGA | | CCUGA | |
| miR-4743 | UGGCCGGAUGGGACAGG | 2561 | AUGCCUCCUGUCCCAUCC | 2562 |
| | AGGCAU | | GGCCA | |
| miR-4744 | UCUAAAGACUAGACUUC | 2563 | CAUAGCGAAGUCUAGUCU | 2564 |
| | GCUAUG | | UUAGA | |
| miR-4745-3p | UGGCCCGGCGACGUCUC | 2565 | GACCGUGAGACGUCGCCG | 2566 |
| | ACGGUC | | GGCCA | |
| miR-4745-5p | UGAGUGGGGCUCCCGGG | 2567 | CGCCGUCCCGGGAGCCCC | 2568 |
| | ACGGCG | | ACUCA | |
| miR-4746-3p | AGCGGUGCUCCUGCGGG | 2569 | UCGGCCCGCAGGAGCACC | 2570 |
| | CCGA | | GCU | |
| miR-4746-5p | CCGGUCCCAGGAGAACC | 2571 | UCUGCAGGUUCUCCUGGG | 2572 |
| | UGCAGA | | ACCGG | |
| miR-4747-3p | AAGGCCCGGGCUUUCCU | 2573 | CUGGGAGGAAAGCCCGGG | 2574 |
| | CCCAG | | CCUU | |
| miR-4747-5p | AGGGAAGGAGGCUUGGU | 2575 | CUAAGACCAAGCCUCCUU | 2576 |
| | CUUAG | | CCCU | |
| miR-4748 | GAGGUUUGGGGAGGAUU | 2577 | AGCAAAUCCUCCCCAAAC | 2578 |
| | UGCU | | CUC | |
| miR-4749-3p | CGCCCCUCCUGCCCCCAC | 2579 | CUGUGGGGGCAGGAGGG | 2580 |
| | AG | | GCG | |
| miR-4749-5p | UGCGGGGACAGGCCAGG | 2581 | GAUGCCCUGGCCUGUCCC | 2582 |
| | GCAUC | | CGCA | |
| miR-4750 | CUCGGGCGGAGGUGGUU | 2583 | CACUCAACCACCUCCGCC | 2584 |
| | GAGUG | | CGAG | |
| miR-4751 | AGAGGACCCGUAGCUGC | 2585 | CCUUCUAGCAGCUACGGG | 2586 |
| | UAGAAGG | | UCCUCU | |
| miR-4752 | UUGUGGAUCUCAAGGAU | 2587 | AGCACAUCCUUGAGAUCC | 2588 |
| | GUGCU | | ACAA | |
| miR-4753-3p | UUCUCUUUCUUUAGCCU | 2589 | ACACAAGGCUAAAGAAAG | 2590 |
| | UGUGU | | AGAA | |
| miR-4753-5p | CAAGGCCAAAGGAAGAG | 2591 | CTGTTCTCTTCCTTTGGCC | 2592 |
| | AACAG | | TTG | |
| miR-4754 | AUGCGGACCUGGGUUAG | 2593 | ACUCCGCUAACCCAGGUC | 2594 |
| | CGGAGU | | CGCAU | |
| miR-4755-3p | AGCCAGGCUCUGAAGGG | 2595 | ACUUUCCCUUCAGAGCCU | 2596 |
| | AAAGU | | GGCU | |
| miR-4755-5p | UUUCCCUUCAGAGCCUG | 2597 | AAAGCCAGGCUCUGAAGG | 2598 |
| | GCUUU | | GAAA | |
| miR-4756-3p | CCAGAGAUGGUUGCCUU | 2599 | AUAGGAAGGCAACCAUCU | 2600 |
| | CCUAU | | CUGG | |
| miR-4756-5p | CAGGGAGGCGCUCACUC | 2601 | AGCAGAGAGUGAGCGCCU | 2602 |
| | UCUGCU | | CCCUG | |
| miR-4757-3p | CAUGACGUCACAGAGGC | 2603 | GCGAAGCCUCUGUGACGU | 2604 |
| | UUCGC | | CAUG | |
| miR-4757-5p | AGGCCUCUGUGACGUCA | 2605 | ACACCGUGACGUCACAGA | 2606 |
| | CGGUGU | | GGCCU | |
| miR-4758-3p | UGCCCCACCUGCUGACC | 2607 | GAGGGUGGUCAGCAGGU | 2608 |
| | ACCCUC | | GGGGCA | |
| miR-4758-5p | GUGAGUGGGAGCCGGUG | 2609 | CAGCCCCACCGGCUCCCA | 2610 |
| | GGGCUG | | CUCAC | |
| miR-4759 | UAGGACUAGAUGUUGGA | 2611 | UAAUUCCAACAUCUAGUC | 2612 |
| | AUUA | | CUA | |
| miR-4760-3p | AAAUUCAUGUUCAAUCU | 2613 | GGUUUAGAUUGAACAUG | 2614 |
| | AAACC | | AAUUU | |
| miR-4760-5p | UUUAGAUUGAACAUGAA | 2615 | CUAACUUCAUGUUCAAUC | 2616 |
| | GUUAG | | UAAA | |
| miR-4761-3p | GAGGGCAUGCGCACUUU | 2617 | GGACAAAGUGCGCAUGCC | 2618 |
| | GUCC | | CUC | |
| miR-4761-5p | ACAAGGUGUGCAUGCCU | 2619 | GGUCAGGCAUGCACACCU | 2620 |
| | GACC | | UGU | |
| miR-4762-3p | CUUCUGAUCAAGAUUUG | 2621 | CACCACAAAUCUUGAUCA | 2622 |
| | UGGUG | | GAAG | |
| miR-4762-5p | CCAAAUCUUGAUCAGAA | 2623 | AGGCUUCUGAUCAAGAUU | 2624 |
| | GCCU | | UGG | |
| miR-4763-3p | AGGCAGGGGCUGGUGCU | 2625 | CCCGCCCAGCACCAGCCC | 2626 |
| | GGGCGGG | | CUGCCU | |
| miR-4763-5p | CGCCUGCCCAGCCCUCCU | 2627 | AGCAGGAGGGCUGGGCAG | 2628 |
| | GCU | | GCG | |
| miR-4764-3p | UUAACUCCUUUCACACC | 2629 | CCAUGGGUGUGAAAGGA | 2630 |
| | CAUGG | | GUUAA | |
| miR-4764-5p | UGGAUGUGGAAGGAGUU | 2631 | AGAUAACUCCUUCCACAU | 2632 |
| | AUCU | | CCA | |
| miR-4765 | UGAGUGAUUGAUAGCUA | 2633 | GAACAUAGCUAUCAAUCA | 2634 |
| | UGUUC | | CUCA | |
| miR-4766-3p | AUAGCAAUUGCUCUUUU | 2635 | UUCCAAAAGAGCAAUUGC | 2636 |
| | GGAA | | UAU | |
| miR-4766-5p | UCUGAAAGAGCAGUUGG | 2637 | AACACCAACUGCUCUUUC | 2638 |
| | UGUU | | AGA | |
| miR-4767 | CGCGGGCGCUCCUGGCC | 2639 | GGCGGCGGCCAGGAGCGC | 2640 |
| | GCCGCC | | CCGCG | |
| miR-4768-3p | CCAGGAGAUCCAGAGAG | 2641 | AUUCUCUCUGGAUCUCCU | 2642 |
| | AAU | | GG | |
| miR-4768-5p | AUUCUCUCUGGAUCCCA | 2643 | AUCCAUGGGAUCCAGAGA | 2644 |
| | UGGAU | | GAAU | |
| miR-4769-3p | UCUGCCAUCCUCCCUCCC | 2645 | GUAGGGGAGGGAGGAUG | 2646 |
| | CUAC | | GCAGA | |
| miR-4769-5p | GGUGGGAUGGAGAGAAG | 2647 | CUCAUACCUUCUCUCCAU | 2648 |
| | GUAUGAG | | CCCACC | |
| miR-4770 | UGAGAUGACACUGUAGC | 2649 | AGCUACAGUGUCAUCUCA | 2650 |
| | U | | | |
| miR-4771 | AGCAGACUUGACCUACA | 2651 | UAAUUGUAGGUCAAGUC | 2652 |
| | AUUA | | UGCU | |
| miR-4772-3p | CCUGCAACUUUGCCUGA | 2653 | UCUGAUCAGGCAAAGUUG | 2654 |
| | UCAGA | | CAGG | |
| miR-4772-5p | UGAUCAGGCAAAAUUGC | 2655 | AGUCUGCAAUUUUGCCUG | 2656 |
| | AGACU | | AUCA | |
| miR-4773 | CAGAACAGGAGCAUAGA | 2657 | GCCUUUCUAUGCUCCUGU | 2658 |
| | AAGGC | | UCUG | |
| miR-4774-3p | AUUGCCUAACAUGUGCC | 2659 | UUCUGGCACAUGUUAGGC | 2660 |
| | AGAA | | AAU | |
| miR-4774-5p | UCUGGUAUGUAGUAGGU | 2661 | UUAUUACCUACUACAUAC | 2662 |
| | AAUAA | | CAGA | |
| miR-4775 | UUAAUUUUUUGUUUCGG | 2663 | AGUGACCGAAACAAAAAA | 2664 |
| | UCACU | | UUAA | |
| miR-4776-3p | CUUGCCAUCCUGGUCCA | 2665 | AUGCAGUGGACCAGGAUG | 2666 |
| | CUGCAU | | GCAAG | |
| miR-4776-5p | GUGGACCAGGAUGGCAA | 2667 | AGCCCUUGCCAUCCUGGU | 2668 |
| | GGGCU | | CCAC | |
| miR-4777-3p | AUACCUCAUCUAGAAUG | 2669 | UACAGCAUUCUAGAUGAG | 2670 |
| | CUGUA | | GUAU | |
| miR-4777-5p | UUCUAGAUGAGAGAUAU | 2671 | UAUAUAUAUCUCUCAUCU | 2672 |
| | AUAUA | | AGAA | |
| miR-4778-3p | UCUUCUUCCUUUGCAGA | 2673 | UCAACUCUGCAAAGGAAG | 2674 |
| | GUUGA | | AAGA | |
| miR-4778-5p | AAUUCUGUAAAGGAAGA | 2675 | CCUCUUCUUCCUUUACAG | 2676 |
| | AGAGG | | AAUU | |
| miR-4779 | UAGGAGGGAAUAGUAAA | 2677 | CUGCUUUUACUAUUCCCU | 2678 |
| | AGCAG | | CCUA | |
| miR-4780 | ACCCUUGAGCCUGAUCC | 2679 | GCUAGGGAUCAGGCUCAA | 2680 |
| | CUAGC | | GGGU | |
| miR-4781-3p | AAUGUUGGAAUCCUCGC | 2681 | CUCUAGCGAGGAUUCCAA | 2682 |
| | UAGAG | | CAUU | |
| miR-4781-5p | UAGCGGGGAUUCCAAUA | 2683 | CCAAUAUUGGAAUCCCCG | 2684 |
| | UUGG | | CUA | |
| miR-4782-3p | UGAUUGUCUUCAUAUCU | 2685 | GUUCUAGAUAUGAAGAC | 2686 |
| | AGAAC | | AAUCA | |
| miR-4782-5p | UUCUGGAUAUGAAGACA | 2687 | UUGAUUGUCUUCAUAUCC | 2688 |
| | AUCAA | | AGAA | |
| miR-4783-3p | CCCCGGUGUUGGGGCGC | 2689 | GCAGACGCGCCCCAACAC | 2690 |
| | GUCUGC | | CGGGG | |
| miR-4783-5p | GGCGCGCCCAGCUCCCG | 2691 | AGCCCGGGAGCUGGGCGC | 2692 |
| | GGCU | | GCC | |
| miR-4784 | UGAGGAGAUGCUGGGAC | 2693 | UCAGUCCCAGCAUCUCCU | 2694 |
| | UGA | | CA | |
| miR-4785 | AGAGUCGGCGACGCCGC | 2695 | GCUGGCGGCGUCGCCGAC | 2696 |
| | CAGC | | UCU | |
| miR-4786-3p | UGAAGCCAGCUCUGGUC | 2697 | GCCCAGACCAGAGCUGGC | 2698 |
| | UGGGC | | UUCA | |
| miR-4786-5p | UGAGACCAGGACUGGAU | 2699 | GGUGCAUCCAGUCCUGGU | 2700 |
| | GCACC | | CUCA | |
| miR-4787-3p | GAUGCGCCGCCCACUGC | 2701 | GCGCGGGGCAGUGGGCGG | 2702 |
| | CCCGCGC | | CGCAUC | |
| miR-4787-5p | GCGGGGGUGGCGGCGGC | 2703 | GGGAUGCCGCCGCCACCC | 2704 |
| | AUCCC | | CCGC | |
| miR-4788 | UUACGGACCAGCUAAGG | 2705 | GCCUCCCUUAGCUGGUCC | 2706 |
| | GAGGC | | GUAA | |
| miR-4789-3p | CACACAUAGCAGGUGUA | 2707 | UAUAUACACCUGCUAUGU | 2708 |
| | UAUA | | GUG | |
| miR-4789-5p | GUAUACACCUGAUAUGU | 2709 | CAUACACAUAUCAGGUGU | 2710 |
| | GUAUG | | AUAC | |
| miR-4790-3p | UGAAUGGUAAAGCGAUG | 2711 | UGUGACAUCGCUUUACCA | 2712 |
| | UCACA | | UUCA | |
| miR-4790-5p | AUCGCUUUACCAUUCAU | 2713 | AACAUGAAUGGUAAAGC | 2714 |
| | GUU | | GAU | |
| miR-4791 | UGGAUAUGAUGACUGAA | 2715 | UUUCAGUCAUCAUAUCCA | 2716 |
| | A | | | |
| miR-4792 | CGGUGAGCGCUCGCUGG | 2717 | GCCAGCGAGCGCUCACCG | 2718 |
| | C | | | |
| miR-4793-3p | UCUGCACUGUGAGUUGG | 2719 | AGCCAGCCAACUCACAGU | 2720 |
| | CUGGCU | | GCAGA | |
| miR-4793-5p | ACAUCCUGCUCCACAGG | 2721 | CCUCUGCCCUGUGGAGCA | 2722 |
| | GCAGAGG | | GGAUGU | |
| miR-4794 | UCUGGCUAUCUCACGAG | 2723 | ACAGUCUCGUGAGAUAGC | 2724 |
| | ACUGU | | CAGA | |
| miR-4795-3p | AUAUUAUUAGCCACUUC | 2725 | AUCCAGAAGUGGCUAAUA | 2726 |
| | UGGAU | | AUAU | |
| miR-4795-5p | AGAAGUGGCUAAUAAUA | 2727 | UCAAUAUUAUUAGCCACU | 2728 |
| | UUGA | | UCU | |
| miR-4796-3p | UAAAGUGGCAGAGUAUA | 2729 | GUGUCUAUACUCUGCCAC | 2730 |
| | GACAC | | UUUA | |
| miR-4796-5p | UGUCUAUACUCUGUCAC | 2731 | GUAAAGUGACAGAGUAU | 2732 |
| | UUUAC | | AGACA | |
| miR-4797-3p | UCUCAGUAAGUGGCACU | 2733 | ACAGAGUGCCACUUACUG | 2734 |
| | CUGU | | AGA | |
| miR-4797-5p | GACAGAGUGCCACUUAC | 2735 | UUCAGUAAGUGGCACUCU | 2736 |
| | UGAA | | GUC | |
| miR-4798-3p | AACUCACGAAGUAUACC | 2737 | ACUUCGGUAUACUUCGUG | 2738 |
| | GAAGU | | AGUU | |
| miR-4798-5p | UUCGGUAUACUUUGUGA | 2739 | CCAAUUCACAAAGUAUAC | 2740 |
| | AUUGG | | CGAA | |
| miR-4799-3p | ACUGGCAUGCUGCAUUU | 2741 | UAUAUAAAUGCAGCAUGC | 2742 |
| | AUAUA | | CAGU | |
| miR-4799-5p | AUCUAAAUGCAGCAUGC | 2743 | GACUGGCAUGCUGCAUUU | 2744 |
| | CAGUC | | AGAU | |
| miR-4800-3p | CAUCCGUCCGUCUGUCC | 2745 | GUGGACAGACGGACGGAU | 2746 |
| | AC | | G | |
| miR-4800-5p | AGUGGACCGAGGAAGGA | 2747 | UCCUUCCUUCCUCGGUCC | 2748 |
| | AGGA | | ACU | |
| miR-4801 | UACACAAGAAAACCAAG | 2749 | UGAGCCUUGGUUUUCUUG | 2750 |
| | GCUCA | | UGUA | |
| miR-4802-3p | UACAUGGAUGGAAACCU | 2751 | GCUUGAAGGUUUCCAUCC | 2752 |
| | UCAAGC | | AUGUA | |
| miR-4802-5p | UAUGGAGGUUCUAGACC | 2753 | AACAUGGUCUAGAACCUC | 2754 |
| | AUGUU | | CAUA | |
| miR-4803 | UAACAUAAUAGUGUGGA | 2755 | UCAAUCCACACUAUUAUG | 2756 |
| | UUGA | | UUA | |
| miR-4804-3p | UGCUUAACCUUGCCCUC | 2757 | UUUCGAGGGCAAGGUUA | 2758 |
| | GAAA | | AGCA | |
| miR-4804-5p | UUGGACGGUAAGGUUAA | 2759 | UUGCUUAACCUUACCGUC | 2760 |
| | GCAA | | CAA | |
| miR-483-3p | UCACUCCUCUCCUCCCG | 2761 | AAGACGGGAGGAGAGGA | 2762 |
| | UCUU | | GUGA | |
| miR-483-5p | AAGACGGGAGGAAAGAA | 2763 | CTCCCTTCTTTCCTCCCGT | 2764 |
| | GGGAG | | CTT | |
| miR-484 | UCAGGCUCAGUCCCCUC | 2765 | AUCGGGAGGGGACUGAGC | 2766 |
| | CCGAU | | CUGA | |
| miR-485-3p | GUCAUACACGGCUCUCC | 2767 | AGAGAGGAGAGCCGUGU | 2768 |
| | UCUCU | | AUGAC | |
| miR-485-5p | AGAGGCUGGCCGUGAUG | 2769 | GAAUUCAUCACGGCCAGC | 2770 |
| | AAUUC | | CUCU | |
| miR-486-3p | CGGGGCAGCUCAGUACA | 2771 | AUCCUGUACUGAGCUGCC | 2772 |
| | GGAU | | CCG | |
| miR-486-5p | UCCUGUACUGAGCUGCC | 2773 | CUCGGGGCAGCUCAGUAC | 2774 |
| | CCGAG | | AGGA | |
| miR-487a | AAUCAUACAGGGACAUC | 2775 | AACUGGAUGUCCCUGUAU | 2776 |
| | CAGUU | | GAUU | |
| miR-487b | AAUCGUACAGGGUCAUC | 2777 | AAGUGGAUGACCCUGUAC | 2778 |
| | CACUU | | GAUU | |
| miR-488-3p | UUGAAAGGCUAUUUCUU | 2779 | GACCAAGAAAUAGCCUUU | 2780 |
| | GGUC | | CAA | |
| miR-488-5p | CCCAGAUAAUGGCACUC | 2781 | UUGAGAGUGCCAUUAUCU | 2782 |
| | UCAA | | GGG | |
| miR-489 | GUGACAUCACAUAUACG | 2783 | GCUGCCGUAUAUGUGAUG | 2784 |
| | GCAGC | | UCAC | |
| miR-490-3p | CAACCUGGAGGACUCCA | 2785 | CAGCAUGGAGUCCUCCAG | 2786 |
| | UGCUG | | GUUG | |
| miR-490-5p | CCAUGGAUCUCCAGGUG | 2787 | ACCCACCUGGAGAUCCAU | 2788 |
| | GGU | | GG | |
| miR-491-3p | CUUAUGCAAGAUUCCCU | 2789 | GUAGAAGGGAAUCUUGC | 2790 |
| | UCUAC | | AUAAG | |
| miR-491-5p | AGUGGGGAACCCUUCCA | 2791 | CCUCAUGGAAGGGUUCCC | 2792 |
| | UGAGG | | CACU | |
| miR-492 | AGGACCUGCGGGACAAG | 2793 | AAGAAUCUUGUCCCGCAG | 2794 |
| | AUUCUU | | GUCCU | |
| miR-493-3p | UGAAGGUCUACUGUGUG | 2795 | CCUGGCACACAGUAGACC | 2796 |
| | CCAGG | | UUCA | |
| miR-493-5p | UUGUACAUGGUAGGCUU | 2797 | AAUGAAAGCCUACCAUGU | 2798 |
| | UCAUU | | ACAA | |
| miR-494 | UGAAACAUACACGGGAA | 2799 | GAGGUUUCCCGUGUAUGU | 2800 |
| | ACCUC | | UUCA | |
| miR-495 | AAACAAACAUGGUGCAC | 2801 | AAGAAGUGCACCAUGUUU | 2802 |
| | UUCUU | | GUUU | |
| miR-496 | UGAGUAUUACAUGGCCA | 2803 | GAGAUUGGCCAUGUAAU | 2804 |
| | AUCUC | | ACUCA | |
| miR-497-3p | CAAACCACACUGUGGUG | 2805 | UCUAACACCACAGUGUGG | 2806 |
| | UUAGA | | UUUG | |
| miR-497-5p | CAGCAGCACACUGUGGU | 2807 | ACAAACCACAGUGUGCUG | 2808 |
| | UUGU | | CUG | |
| miR-498 | UUUCAAGCCAGGGGGCG | 2809 | GAAAAACGCCCCCUGGCU | 2810 |
| | UUUUUC | | UGAAA | |
| miR-4999-3p | UCACUACCUGACAAUAC | 2811 | ACUGUAUUGUCAGGUAG | 2812 |
| | AGU | | UGA | |
| miR-4999-5p | UGCUGUAUUGUCAGGUA | 2813 | UCACUACCUGACAAUACA | 2814 |
| | GUGA | | GCA | |
| miR-499a-3p | AACAUCACAGCAAGUCU | 2815 | AGCACAGACUUGCUGUGA | 2816 |
| | GUGCU | | UGUU | |
| miR-499a-5p | UUAAGACUUGCAGUGAU | 2817 | AAACAUCACUGCAAGUCU | 2818 |
| | GUUU | | UAA | |
| miR-499b-3p | AACAUCACUGCAAGUCU | 2819 | UGUUAAGACUUGCAGUG | 2820 |
| | UAACA | | AUGUU | |
| miR-499b-5p | ACAGACUUGCUGUGAUG | 2821 | UGAACAUCACAGCAAGUC | 2822 |
| | UUCA | | UGU | |
| miR-5000-3p | UCAGGACACUUCUGAAC | 2823 | UCCAAGUUCAGAAGUGUC | 2824 |
| | UUGGA | | CUGA | |
| miR-5000-5p | CAGUUCAGAAGUGUUCC | 2825 | ACUCAGGAACACUUCUGA | 2826 |
| | UGAGU | | ACUG | |
| miR-5001-3p | UUCUGCCUCUGUCCAGG | 2827 | AAGGACCUGGACAGAGGC | 2828 |
| | UCCUU | | AGAA | |
| miR-5001-5p | AGGGCUGGACUCAGCGG | 2829 | AGCUCCGCCGCUGAGUCC | 2830 |
| | CGGAGCU | | AGCCCU | |
| miR-5002-3p | UGACUGCCUCACUGACC | 2831 | AAGUGGUCAGUGAGGCA | 2832 |
| | ACUU | | GUCA | |
| miR-5002-5p | AAUUUGGUUUCUGAGGC | 2833 | ACUAAGUGCCUCAGAAAC | 2834 |
| | ACUUAGU | | CAAAUU | |
| miR-5003-3p | UACUUUUCUAGGUUGUU | 2835 | CCCCAACAACCUAGAAAA | 2836 |
| | GGGG | | GUA | |
| miR-5003-5p | UCACAACAACCUUGCAG | 2837 | UCUACCCUGCAAGGUUGU | 2838 |
| | GGUAGA | | UGUGA | |
| miR-5004-3p | CUUGGAUUUUCCUGGGC | 2839 | CUGAGGCCCAGGAAAAUC | 2840 |
| | CUCAG | | CAAG | |
| miR-5004-5p | UGAGGACAGGGCAAAUU | 2841 | UCGUGAAUUUGCCCUGUC | 2842 |
| | CACGA | | CUCA | |
| miR-5006-3p | UUUCCCUUUCCAUCCUG | 2843 | CUGCCAGGAUGGAAAGGG | 2844 |
| | GCAG | | AAA | |
| miR-5006-5p | UUGCCAGGGCAGGAGGU | 2845 | UUCCACCUCCUGCCCUGG | 2846 |
| | GGAA | | CAA | |
| miR-5007-3p | AUCAUAUGAACCAAACU | 2847 | AUUAGAGUUUGGUUCAU | 2848 |
| | CUAAU | | AUGAU | |
| miR-5007-5p | UAGAGUCUGGCUGAUAU | 2849 | AAACCAUAUCAGCCAGAC | 2850 |
| | GGUUU | | UCUA | |
| miR-5008-3p | CCUGUGCUCCCAGGGCC | 2851 | GCGAGGCCCUGGGAGCAC | 2852 |
| | UCGC | | AGG | |
| miR-5008-5p | UGAGGCCCUUGGGGCAC | 2853 | CCACUGUGCCCCAAGGGC | 2854 |
| | AGUGG | | CUCA | |
| miR-5009-3p | UCCUAAAUCUGAAAGUC | 2855 | UUUUGGACUUUCAGAUU | 2856 |
| | CAAAA | | UAGGA | |
| miR-5009-5p | UUGGACUUUUUCAGAUU | 2857 | AUCCCCAAAUCUGAAAAA | 2858 |
| | UGGGGAU | | GUCCAA | |
| miR-500a-3p | AUGCACCUGGGCAAGGA | 2859 | CAGAAUCCUUGCCCAGGU | 2860 |
| | UUCUG | | GCAU | |
| miR-500a-5p | UAAUCCUUGCUACCUGG | 2861 | UCUCACCCAGGUAGCAAG | 2862 |
| | GUGAGA | | GAUUA | |
| miR-500b | AAUCCUUGCUACCUGGG | 2863 | ACCCAGGUAGCAAGGAUU | 2864 |
| | U | | | |
| miR-501-3p | AAUGCACCCGGGCAAGG | 2865 | AGAAUCCUUGCCCGGGUG | 2866 |
| | AUUCU | | CAUU | |
| miR-501-5p | AAUCCUUUGUCCCUGGG | 2867 | UCUCACCCAGGGACAAAG | 2868 |
| | UGAGA | | GAUU | |
| miR-5010-3p | UUUUGUGUCUCCCAUUC | 2869 | CUGGGGAAUGGGAGACAC | 2870 |
| | CCCAG | | AAAA | |
| miR-5010-5p | AGGGGGAUGGCAGAGCA | 2871 | AAUUUUGCUCUGCCAUCC | 2872 |
| | AAAUU | | CCCU | |
| miR-5011-3p | GUGCAUGGCUGUAUAUA | 2873 | UGUUAUAUAUACAGCCAU | 2874 |
| | UAACA | | GCAC | |
| miR-5011-5p | UAUAUAUACAGCCAUGC | 2875 | GAGUGCAUGGCUGUAUA | 2876 |
| | ACUC | | UAUA | |
| miR-502-3p | AAUGCACCUGGGCAAGG | 2877 | UGAAUCCUUGCCCAGGUG | 2878 |
| | AUUCA | | CAUU | |
| miR-502-5p | AUCCUUGCUAUCUGGGU | 2879 | UAGCACCCAGAUAGCAAG | 2880 |
| | GCUA | | GAU | |
| miR-503 | UAGCAGCGGGAACAGUU | 2881 | CUGCAGAACUGUUCCCGC | 2882 |
| | CUGCAG | | UGCUA | |
| miR-504 | AGACCCUGGUCUGCACU | 2883 | GAUAGAGUGCAGACCAGG | 2884 |
| | CUAUC | | GUCU | |
| miR-5047 | UUGCAGCUGCGGUUGUA | 2885 | ACCUUACAACCGCAGCUG | 2886 |
| | AGGU | | CAA | |
| miR-505-3p | CGUCAACACUUGCUGGU | 2887 | AGGAAACCAGCAAGUGUU | 2888 |
| | UUCCU | | GACG | |
| miR-505-5p | GGGAGCCAGGAAGUAUU | 2889 | ACAUCAAUACUUCCUGGC | 2890 |
| | GAUGU | | UCCC | |
| miR-506-3p | UAAGGCACCCUUCUGAG | 2891 | UCUACUCAGAAGGGUGCC | 2892 |
| | UAGA | | UUA | |
| miR-506-5p | UAUUCAGGAAGGUGUUA | 2893 | UUAAGUAACACCUUCCUG | 2894 |
| | CUUAA | | AAUA | |
| miR-507 | UUUUGCACCUUUUGGAG | 2895 | UUCACUCCAAAAGGUGCA | 2896 |
| | UGAA | | AAA | |
| miR-508-3p | UGAUUGUAGCCUUUUGG | 2897 | UCUACUCCAAAAGGCUAC | 2898 |
| | AGUAGA | | AAUCA | |
| miR-508-5p | UACUCCAGAGGGCGUCA | 2899 | CAUGAGUGACGCCCUCUG | 2900 |
| | CUCAUG | | GAGUA | |
| miR-5087 | GGGUUUGUAGCUUUGCU | 2901 | CAUGCCAGCAAAGCUACA | 2902 |
| | GGCAUG | | AACCC | |
| miR-5088 | CAGGGCUCAGGGAUUGG | 2903 | CUCCAUCCAAUCCCUGAG | 2904 |
| | AUGGAG | | CCCUG | |
| miR-5089 | GUGGGAUUUCUGAGUAG | 2905 | GAUGCUACUCAGAAAUCC | 2906 |
| | CAUC | | CAC | |
| miR-509-3-5p | UACUGCAGACGUGGCAA | 2907 | CAUGAUUGCCACGUCUGC | 2908 |
| | UCAUG | | AGUA | |
| miR-509-3p | UGAUUGGUACGUCUGUG | 2909 | CUACCCACAGACGUACCA | 2910 |
| | GGUAG | | AUCA | |
| miR-509-5p | UACUGCAGACAGUGGCA | 2911 | UGAUUGCCACUGUCUGCA | 2912 |
| | AUCA | | GUA | |
| miR-5090 | CCGGGGCAGAUUGGUGU | 2913 | CACCCUACACCAAUCUGC | 2914 |
| | AGGGUG | | CCCGG | |
| miR-5091 | ACGGAGACGACAAGACU | 2915 | CAGCACAGUCUUGUCGUC | 2916 |
| | GUGCUG | | UCCGU | |
| miR-5092 | AAUCCACGCUGAGCUUG | 2917 | GAUGCCAAGCUCAGCGUG | 2918 |
| | GCAUC | | GAUU | |
| miR-5093 | AGGAAAUGAGGCUGGCU | 2919 | GCUCCUAGCCAGCCUCAU | 2920 |
| | AGGAGC | | UUCCU | |
| miR-5094 | AAUCAGUGAAUGCCUUG | 2921 | AGGUUCAAGGCAUUCACU | 2922 |
| | AACCU | | GAUU | |
| miR-5095 | UUACAGGCGUGAACCAC | 2923 | CGCGGUGGUUCACGCCUG | 2924 |
| | CGCG | | UAA | |
| miR-5096 | GUUUCACCAUGUUGGUC | 2925 | GCCUGACCAACAUGGUGA | 2926 |
| | AGGC | | AAC | |
| miR-510 | UACUCAGGAGAGUGGCA | 2927 | GUGAUUGCCACUCUCCUG | 2928 |
| | AUCAC | | AGUA | |
| miR-5100 | UUCAGAUCCCAGCGGUG | 2929 | AGAGGCACCGCUGGGAUC | 2930 |
| | CCUCU | | UGAA | |
| miR-511 | GUGUCUUUUGCUCUGCA | 2931 | UGACUGCAGAGCAAAAGA | 2932 |
| | GUCA | | CAC | |
| miR-512-3p | AAGUGCUGUCAUAGCUG | 2933 | GACCUCAGCUAUGACAGC | 2934 |
| | AGGUC | | ACUU | |
| miR-512-5p | CACUCAGCCUUGAGGGC | 2935 | GAAAGUGCCCUCAAGGCU | 2936 |
| | ACUUUC | | GAGUG | |
| miR-513a-3p | UAAAUUUCACCUUUCUG | 2937 | CCUUCUCAGAAAGGUGAA | 2938 |
| | AGAAGG | | AUUUA | |
| miR-513a-5p | UUCACAGGGAGGUGUCA | 2939 | AUGACACCUCCCUGUGAA | 2940 |
| | U | | | |
| miR-513b | UUCACAAGGAGGUGUCA | 2941 | AUAAAUGACACCUCCUUG | 2942 |
| | UUUAU | | UGAA | |
| miR-513c-3p | UAAAUUUCACCUUUCUG | 2943 | UCUUCUCAGAAAGGUGAA | 2944 |
| | AGAAGA | | AUUUA | |
| miR-513c-5p | UUCUCAAGGAGGUGUCG | 2945 | AUAAACGACACCUCCUUG | 2946 |
| | UUUAU | | AGAA | |
| miR-514a-3p | AUUGACACUUCUGUGAG | 2947 | UCUACUCACAGAAGUGUC | 2948 |
| | UAGA | | AAU | |
| miR-514a-5p | UACUCUGGAGAGUGACA | 2949 | CAUGAUUGUCACUCUCCA | 2950 |
| | AUCAUG | | GAGUA | |
| miR-514b-3p | AUUGACACCUCUGUGAG | 2951 | UCCACUCACAGAGGUGUC | 2952 |
| | UGGA | | AAU | |
| miR-514b-5p | UUCUCAAGAGGGAGGCA | 2953 | AUGAUUGCCUCCCUCUUG | 2954 |
| | AUCAU | | AGAA | |
| miR-515-3p | GAGUGCCUUCUUUUGGA | 2955 | AACGCUCCAAAAGAAGGC | 2956 |
| | GCGUU | | ACUC | |
| miR-515-5p | UUCUCCAAAAGAAAGCA | 2957 | CAGAAAGUGCUUUCUUUU | 2958 |
| | CUUUCUG | | GGAGAA | |
| miR-516a-3p | UGCUUCCUUUCAGAGGG | 2959 | ACCCUCUGAAAGGAAGCA | 2960 |
| | U | | | |
| miR-516a-5p | UUCUCGAGGAAAGAAGC | 2961 | GAAAGUGCUUCUUUCCUC | 2962 |
| | ACUUUC | | GAGAA | |
| miR-516b-3p | UGCUUCCUUUCAGAGGG | 2963 | ACCCUCUGAAAGGAAGCA | 2964 |
| | U | | | |
| miR-516b-5p | AUCUGGAGGUAAGAAGC | 2965 | AAAGUGCUUCUUACCUCC | 2966 |
| | ACUUU | | AGAU | |
| miR-517-5p | CCUCUAGAUGGAAGCAC | 2967 | AGACAGUGCUUCCAUCUA | 2968 |
| | UGUCU | | GAGG | |
| miR-517a-3p | AUCGUGCAUCCCUUUAG | 2969 | ACACUCUAAAGGGAUGCA | 2970 |
| | AGUGU | | CGAU | |
| miR-517b-3p | AUCGUGCAUCCCUUUAG | 2971 | ACACUCUAAAGGGAUGCA | 2972 |
| | AGUGU | | CGAU | |
| miR-517c-3p | AUCGUGCAUCCUUUUAG | 2973 | ACACUCUAAAAGGAUGCA | 2974 |
| | AGUGU | | CGAU | |
| miR-5186 | AGAGAUUGGUAGAAAUC | 2975 | ACCUGAUUUCUACCAAUC | 2976 |
| | AGGU | | UCU | |
| miR-5187-3p | ACUGAAUCCUCUUUUCC | 2977 | CUGAGGAAAAGAGGAUU | 2978 |
| | UCAG | | CAGU | |
| miR-5187-5p | UGGGAUGAGGGAUUGAA | 2979 | UCCACUUCAAUCCCUCAU | 2980 |
| | GUGGA | | CCCA | |
| miR-5188 | AAUCGGACCCAUUUAAA | 2981 | CUCCGGUUUAAAUGGGUC | 2982 |
| | CCGGAG | | CGAUU | |
| miR-5189 | UCUGGGCACAGGCGGAU | 2983 | CCUGUCCAUCCGCCUGUG | 2984 |
| | GGACAGG | | CCCAGA | |
| miR-518a-3p | GAAAGCGCUUCCCUUUG | 2985 | UCCAGCAAAGGGAAGCGC | 2986 |
| | CUGGA | | UUUC | |
| miR-518a-5p | CUGCAAAGGGAAGCCCU | 2987 | GAAAGGGCUUCCCUUUGC | 2988 |
| | UUC | | AG | |
| miR-518b | CAAAGCGCUCCCCUUUA | 2989 | ACCUCUAAAGGGGAGCGC | 2990 |
| | GAGGU | | UUUG | |
| miR-518c-3p | CAAAGCGCUUCUCUUUA | 2991 | ACACUCUAAAGAGAAGCG | 2992 |
| | GAGUGU | | CUUUG | |
| miR-518c-5p | UCUCUGGAGGGAAGCAC | 2993 | CAGAAAGUGCUUCCCUCC | 2994 |
| | UUUCUG | | AGAGA | |
| miR-518d-3p | CAAAGCGCUUCCCUUUG | 2995 | GCUCCAAAGGGAAGCGCU | 2996 |
| | GAGC | | UUG | |
| miR-518d-5p | CUCUAGAGGGAAGCACU | 2997 | CAGAAAGUGCUUCCCUCU | 2998 |
| | UUCUG | | AGAG | |
| miR-518e-3p | AAAGCGCUUCCCUUCAG | 2999 | CACUCUGAAGGGAAGCGC | 3000 |
| | AGUG | | UUU | |
| miR-518e-5p | CUCUAGAGGGAAGCGCU | 3001 | CAGAAAGCGCUUCCCUCU | 3002 |
| | UUCUG | | AGAG | |
| miR-518f-3p | GAAAGCGCUUCUCUUUA | 3003 | CCUCUAAAGAGAAGCGCU | 3004 |
| | GAGG | | UUC | |
| miR-518f-5p | CUCUAGAGGGAAGCACU | 3005 | GAGAAAGUGCUUCCCUCU | 3006 |
| | UUCUC | | AGAG | |
| miR-5190 | CCAGUGACUGAGCUGGA | 3007 | UGGCUCCAGCUCAGUCAC | 3008 |
| | GCCA | | UGG | |
| miR-5191 | AGGAUAGGAAGAAUGAA | 3009 | AGCACUUCAUUCUUCCUA | 3010 |
| | GUGCU | | UCCU | |
| miR-5192 | AGGAGAGUGGAUUCCAG | 3011 | ACCACCUGGAAUCCACUC | 3012 |
| | GUGGU | | UCCU | |
| miR-5193 | UCCUCCUCUACCUCAUC | 3013 | ACUGGGAUGAGGUAGAG | 3014 |
| | CCAGU | | GAGGA | |
| miR-5194 | UGAGGGGUUUGGAAUGG | 3015 | CCAUCCCAUUCCAAACCC | 3016 |
| | GAUGG | | CUCA | |
| miR-5195-3p | AUCCAGUUCUCUGAGGG | 3017 | AGCCCCCUCAGAGAACUG | 3018 |
| | GGCU | | GAU | |
| miR-5195-5p | AACCCCUAAGGCAACUG | 3019 | CCAUCCAGUUGCCUUAGG | 3020 |
| | GAUGG | | GGUU | |
| miR-5196-3p | UCAUCCUCGUCUCCCUC | 3021 | CUGGGAGGGAGACGAGG | 3022 |
| | CCAG | | AUGA | |
| miR-5196-5p | AGGGAAGGGGACGAGGG | 3023 | CCCAACCCUCGUCCCCUU | 3024 |
| | UUGGG | | CCCU | |
| miR-5197-3p | AAGAAGAGACUGAGUCA | 3025 | AUUCGAUGACUCAGUCUC | 3026 |
| | UCGAAU | | UUCUU | |
| miR-5197-5p | CAAUGGCACAAACUCAU | 3027 | UCAAGAAUGAGUUUGUG | 3028 |
| | UCUUGA | | CCAUUG | |
| miR-519a-3p | AAAGUGCAUCCUUUUAG | 3029 | ACUACUCUAAAAGGAUGCA | 3030 |
| | AGUGU | | CUUU | |
| miR-519a-5p | CUCUAGAGGGAAGCGCU | 3031 | CAGAAAGCGCUUCCCUCU | 3032 |
| | UUCUG | | AGAG | |
| miR-519b-3p | AAAGGCAUCCUUUUAG | 3033 | AACCUCUAAAAGGAUGCA | 3034 |
| | AGGUU | | CUUU | |
| miR-519b-5p | CUCUAGAGGGAAGCGCU | 3035 | CAGAAAGCGCUUCCCUCU | 3036 |
| | UUCUG | | AGAG | |
| miR-519c-3p | AAAGUGCAUCUUUUUAG | 3037 | AUCCUCUAAAAAGAUGCA | 3038 |
| | AGGAU | | CUUU | |
| miR-519c-5p | CUCUAGAGGGAAGCGCU | 3039 | CAGAAAGCGCUUCCCUCU | 3040 |
| | UUCUG | | AGAG | |
| miR-519d | CAAAGUGCCUCCCUUUA | 3041 | CACUCUAAAGGGAGGCAC | 3042 |
| | GAGUG | | UUUG | |
| miR-519e-3p | AAGUGCCUCCUUUUAGA | 3043 | AACACUCUAAAAGGAGGC | 3044 |
| | GUGUU | | ACUU | |
| miR-519e-5p | UUCUCCAAAAGGGAGCA | 3045 | GAAAGUGCUCCCUUUUGG | 3046 |
| | CUUUC | | AGAA | |
| miR-520a-3p | AAAGUGCUUCCCUUUGG | 3047 | ACAGUCCAAAGGGAAGCA | 3048 |
| | AGUGU | | CUUU | |
| miR-520a-5p | CUCCAGAGGGAAGUACU | 3049 | AGAAAGUACUUCCCUCUG | 3050 |
| | UUCU | | GAG | |
| miR-520b | AAAGUGCUUCCUUUUAG | 3051 | CCCUCUAAAAGGAAGCAC | 3052 |
| | AGGG | | UUU | |
| miR-520c-3p | AAAGUGCUUCCUUUUAG | 3053 | ACCCUCUAAAAGGAAGCA | 3054 |
| | AGGGU | | CUUU | |
| miR-520c-5p | CUCUAGAGGGAAGCACU | 3055 | CAGAAAGUGCUUCCCUCU | 3056 |
| | UUCUG | | AGAG | |
| miR-520d-3p | AAAGUGCUUCUCUUUGG | 3057 | ACCCACCAAAGAGAAGCA | 3058 |
| | UGGGU | | CUUU | |
| miR-520d-5p | CUACAAAGGGAAGCCCU | 3059 | GAAAGGGCUUCCCUUUGU | 3060 |
| | UUC | | AG | |
| miR-520e | AAAGUGCUUCCUUUUUG | 3061 | CCCUCAAAAAGGAAGCAC | 3062 |
| | AGGG | | UUU | |
| miR-520f | AAGUGCUUCCUUUUAGA | 3063 | AACCCUCUAAAAGGAAGC | 3064 |
| | GGGUU | | ACUU | |
| miR-520g | ACAAAGUGCUUCCCUUU | 3065 | ACACUCUAAAGGGAAGCA | 3066 |
| | AGAGUGU | | CUUUGU | |
| miR-520h | ACAAAGUGCUUCCCUUU | 3067 | ACUCUAAAGGGAAGCACU | 3068 |
| | AGAGU | | UUGU | |
| miR-521 | AACGCACUUCCCUUUAG | 3069 | ACACUCUAAAGGGAAGUG | 3070 |
| | AGUGU | | CGUU | |
| miR-522-3p | AAAAUGGUUCCCUUUAG | 3071 | ACACUCUAAAGGGAACCA | 3072 |
| | AGUGU | | UUUU | |
| miR-522-5p | CUCUAGAGGGAAGCGCU | 3073 | CAGAAAGCGCUUCCCUCU | 3074 |
| | UUCUG | | AGAG | |
| miR-523-3p | GAACGCGCUUCCCUAUA | 3075 | ACCCUCUAUAGGGAAGCG | 3076 |
| | GAGGGU | | CGUUC | |
| miR-523-5p | CUCUAGAGGGAAGCGCU | 3077 | CAGAAAGCGCUUCCCUCU | 3078 |
| | UUCUG | | AGAG | |
| miR-524-3p | GAAGGCGCUUCCCUUUG | 3079 | ACUCCAAAGGGAAGCGCC | 3080 |
| | GAGU | | UUC | |
| miR-524-5p | CUACAAAGGGAAGCACU | 3081 | GAGAAAGUGCUUCCCUUU | 3082 |
| | UUCUC | | GUAG | |
| miR-525-3p | GAAGGCGCUUCCCUUUA | 3083 | CGCUCUAAAGGGAAGCGC | 3084 |
| | GAGCG | | CUUC | |
| miR-525-5p | CUCCAGAGGGAUGCACU | 3085 | AGAAAGUGCAUCCCUCUG | 3086 |
| | UUCU | | GAG | |
| miR-526a | CUCUAGAGGGAAGCACU | 3087 | CAGAAAGUGCUUCCCUCU | 3088 |
| | UUCUG | | AGAG | |
| miR-526b-3p | GAAAGUGCUUCCUUUUA | 3089 | GCCUCUAAAAGGAAGCAC | 3090 |
| | GAGGC | | UUUC | |
| miR-526b-5p | CUCUUGAGGGAAGCACU | 3091 | ACAGAAAGUGCUUCCCUC | 3092 |
| | UUCUGU | | AAGAG | |
| miR-527 | CUGCAAAGGGAAGCCCU | 3093 | GAAAGGGCUUCCCUUUGC | 3094 |
| | UUC | | AG | |
| miR-532-3p | CCUCCCACACCCAAGGC | 3095 | UGCAAGCCUUGGGUGUGG | 3096 |
| | UUGCA | | GAGG | |
| miR-532-5p | CAUGCCUUGAGUGUAGG | 3097 | ACGGUCCUACACUCAAGG | 3098 |
| | ACCGU | | CAUG | |
| miR-539-3p | AUCAUACAAGGACAAUU | 3099 | AAAGAAAUUGUCCUUGU | 3100 |
| | UCUUU | | AUGAU | |
| miR-539-5p | GGAGAAAUUAUCCUUGG | 3101 | ACACACCAAGGAUAAUUU | 3102 |
| | UGUGU | | CUCC | |
| miR-541-3p | UGGUGGGCACAGAAUCU | 3103 | AGUCCAGAUUCUGUGCCC | 3104 |
| | GGACU | | ACCA | |
| miR-541-5p | AAAGGAUUCUGCUGUCG | 3105 | AGUGGGACCGACAGCAGA | 3106 |
| | GUCCCACU | | AUCCUUU | |
| miR-542-3p | UGUGACAGAUUGAUAAC | 3107 | UUUCAGUUAUCAAUCUGU | 3108 |
| | UGAAA | | CACA | |
| miR-542-5p | UCGGGGAUCAUCAUGUC | 3109 | UCUCGUGACAUGAUGAUC | 3110 |
| | ACGAGA | | CCCGA | |
| miR-543 | AAACAUUCGCGGUGCAC | 3111 | AAGAAGUGCACCGCGAAU | 3112 |
| | UUCUU | | GUUU | |
| miR-544a | AUUCUGCAUUUUUAGCA | 3113 | GAACUUGCUAAAAAUGCA | 3114 |
| | AGUUC | | GAAU | |
| miR-544b | ACCUGAGGUUGUGCAUU | 3115 | UUAGAAAUGCACAACCUC | 3116 |
| | UCUAA | | AGGU | |
| miR-545-3p | UCAGCAAACAUUUAUUG | 3117 | GCACACAAUAAAUGUUUG | 3118 |
| | UGUGC | | CUGA | |
| miR-545-5p | UCAGUAAAUGUUUAUUA | 3119 | UCAUCUAAUAAACAUUUA | 3120 |
| | GAUGA | | CUGA | |
| miR-548a-3p | CAAAACUGGCAAUUACU | 3121 | GCAAAAGUAAUUGCCAGU | 3122 |
| | UUUGC | | UUUG | |
| miR-548a-5p | AAAAGUAAUUGCGAGUU | 3123 | GGUAAAACUCGCAAUUAC | 3124 |
| | UUACC | | UUUU | |
| miR-548aa | AAAAACCACAAUUACUU | 3125 | UGGUGCAAAAGUAAUUG | 3126 |
| | UUGCACCA | | UGGUUUUU | |
| miR-548ab | AAAAGUAAUUGUGGAUU | 3127 | AGCAAAAUCCACAAUUAC | 3128 |
| | UUGCU | | UUUU | |
| miR-548ac | CAAAAACCGGCAAUUAC | 3129 | CAAAAGUAAUUGCCGGUU | 3130 |
| | UUUUG | | UUUG | |
| miR-548ad | GAAAACGACAAUGACUU | 3131 | UGCAAAAGUCAUUGUCGU | 3132 |
| | UUGCA | | UUUC | |
| miR-548ae | CAAAAACUGCAAUUACU | 3133 | UGAAAGUAAUUGCAGUU | 3134 |
| | UUCA | | UUUG | |
| miR-548ag | AAAGGUAAUUGUGGUUU | 3135 | GCAGAAACCACAAUUACC | 3136 |
| | CUGC | | UUU | |
| miR-548ah-3p | CAAAAACUGCAGUUACU | 3137 | GCAAAAGUAACUGCAGUU | 3138 |
| | UUUGC | | UUUG | |
| miR-548ah-5p | AAAAGUGAUUGCAGUGU | 3139 | CAAACACUGCAAUCACUU | 3140 |
| | UUG | | UU | |
| miR-548ai | AAAGGUAAUUGCAGUUU | 3141 | GGGAAAAACUGCAAUUAC | 3142 |
| | UUCCC | | CUUU | |
| miR-548aj-3p | UAAAAACUGCAAUUACU | 3143 | UAAAAGUAAUUGCAGUU | 3144 |
| | UUUA | | UUUA | |
| miR-548aj-5p | UGCAAAAGUAAUUGCAG | 3145 | CAAAAACUGCAAUUACUU | 3146 |
| | UUUUUG | | UUGCA | |
| miR-548ak | AAAAGUAACUGCGGUUU | 3147 | UCAAAAACCGCAGUUACU | 3148 |
| | UUGA | | UUU | |
| miR-548al | AACGGCAAUGACUUUUG | 3149 | UGGUACAAAAGUCAUUGC | 3150 |
| | UACCA | | CGUU | |
| miR-548am-3p | CAAAAACUGCAGUUACU | 3151 | ACAAAAGUAACUGCAGUU | 3152 |
| | UUUGU | | UUUG | |
| miR-548am-5p | AAAAGUAAUUGCGGUUU | 3153 | GGCAAAAACCGCAAUUAC | 3154 |
| | UUGCC | | UUUU | |
| miR-548an | AAAAGGCAUUGUGGUUU | 3155 | CAAAAACCACAAUGCCUU | 3156 |
| | UUG | | UU | |
| miR-548ao-3p | AAAGACCGUGACUACUU | 3157 | UGCAAAAGUAGUCACGGU | 3158 |
| | UUGCA | | CUUU | |
| miR-548ao-5p | AGAAGUAACUACGGUUU | 3159 | UGCAAAAACCGUAGUUAC | 3160 |
| | UUGCA | | UUCU | |
| miR-548ap-3pu | AAAAACCACAAUUACUUu | 3161 | AAAAGUAAUUGUGGUUU | 3162 |
| | UU | | UU | |
| miR-548ap-5puu | AAAAGUAAUUGCGGUCU | 3163 | AAAGACCGCAAUUACUUU | 3164 |
| | UU | | U | |
| miR-548aq-3p | CAAAAACUGCAAUUACU | 3165 | GCAAAAGUAAUUGCAGU | 3166 |
| | UUUGC | | UUUUG | |
| miR-548aq-5p | GAAAGUAAUUGCUGUUU | 3167 | GGCAAAAACAGCAAUUAC | 3168 |
| | UUGCC | | UUUC | |
| miR-548ar-3p | UAAAACUGCAGUUAUUU | 3169 | GCAAAAAUAACUGCAGUU | 3170 |
| | UUGC | | UUA | |
| miR-548ar-5p | AAAAGUAAUUGCAGUUU | 3171 | GCAAAAACUGCAAUUACU | 3172 |
| | UUGC | | UUU | |
| miR-548as-3p | UAAAACCCACAAUUAUG | 3173 | ACAAACAUAAUUGUGGG | 3174 |
| | UUUGU | | UUUUA | |
| miR-548as-5p | AAAAGUAAUUGCGGGUU | 3175 | GGCAAAACCCGCAAUUAC | 3176 |
| | UUGCC | | UUUU | |
| miR-548at-3p | CAAAACCGCAGUAACUU | 3177 | ACAAAAGUUACUGCGGUU | 3178 |
| | UUGU | | UUG | |
| miR-548at-5p | AAAAGUUAUUGCGGUUU | 3179 | AGCCAAAACCGCAAUAAC | 3180 |
| | UGGCU | | UUUU | |
| miR-548au-3p | UGGCAGUUACUUUUGCA | 3181 | CUGGUGCAAAAGUAACUG | 3182 |
| | CCAG | | CCA | |
| miR-548au-5p | AAAAGUAAUUGCGGUUU | 3183 | GCAAAAACCGCAAUUACU | 3184 |
| | UUGC | | UUU | |
| miR-548av-3p | AAACUGCAGUUACUUU | 3185 | GCAAAAGUAACUGCAGUU | 3186 |
| | UGC | | UU | |
| miR-548av-5p | AAAAGUACUUGCGGAUU | 3187 | AAAUCCGCAAGUACUUUU | 3188 |
| | U | | | |
| miR-548aw | GUGCAAAAGUCAUCACG | 3189 | AACCGUGAUGACUUUUGC | 3190 |
| | GUU | | AC | |
| miR-548ax | AGAAGUAAUUGCGGUUU | 3191 | UGGCAAAACCGCAAUUAC | 3192 |
| | UGCCA | | UUCU | |
| miR-548b-3p | CAAGAACCUCAGUUGCU | 3193 | ACAAAAGCAACUGAGGUU | 3194 |
| | UUUGU | | CUUG | |
| miR-548b-5p | AAAAGUAAUUGUGGUUU | 3195 | GGCCAAAACCACAAUUAC | 3196 |
| | UGGCC | | UUUU | |
| miR-548c-3p | CAAAAAUCUCAAUUACU | 3197 | GCAAAAGUAAUUGAGAU | 3198 |
| | UUUGC | | UUUUG | |
| miR-548c-5p | AAAAGUAAUUGCGGUUU | 3199 | GGCAAAAACCGCAAUUAC | 3200 |
| | UUGCC | | UUUU | |
| miR-548d-3p | CAAAAACCACAGUUUCU | 3201 | GCAAAAGAAACUGUGGU | 3202 |
| | UUUGC | | UUUUG | |
| miR-548d-5p | AAAAGUAAUUGUGGUUU | 3203 | GGCAAAAACCACAAUUAC | 3204 |
| | UUGCC | | UUUU | |
| miR-548e | AAAAACUGAGACUACUU | 3205 | UGCAAAAGUAGUCUCAGU | 3206 |
| | UUGCA | | UUUU | |
| miR-548f | AAAAACUGUAAUUACUU | 3207 | AAAAGUAAUUACAGUUU | 3208 |
| | UU | | UU | |
| miR-548g-3p | AAAACUGUAAUUACUUU | 3209 | GUACAAAAGUAAUUACA | 3210 |
| | UGUAC | | GUUUU | |
| miR-548g-5p | UGCAAAAGUAAUUGCAG | 3211 | CAAAAACUGCAAUUACUU | 3212 |
| | UUUUUG | | UUGCA | |
| miR-548h-3p | CAAAAACCGCAAUUACU | 3213 | UGCAAAAGUAAUUGCGG | 3214 |
| | UUUGCA | | UUUUUG | |
| miR-548h-5p | AAAAGUAAUCGCGGUUU | 3215 | GACAAAAACCGCGAUUAC | 3216 |
| | UUGUC | | UUUU | |
| miR-548i | AAAAGUAAUUGCGGAUU | 3217 | GGCAAAAUCCGCAAUUAC | 3218 |
| | UUGCC | | UUUU | |
| miR-548j | AAAAGUAAUUGCGGUCU | 3219 | ACCAAAGACCGCAAUUAC | 3220 |
| | UUGGU | | UUUU | |
| miR-548k | AAAAGUACUUGCGGAUU | 3221 | AGCAAAAUCCGCAAGUAC | 3222 |
| | UUGCU | | UUUU | |
| miR-548l | AAAAGUAUUUGCGGGUU | 3223 | GACAAAACCCGCAAAUAC | 3224 |
| | UUGUC | | UUUU | |
| miR-548m | CAAAGGUAUUUGUGGUU | 3225 | CAAAAACCACAAAUACCU | 3226 |
| | UUUG | | UUG | |
| miR-548n | CAAAAGUAAUUGUGGAU | 3227 | ACAAAAUCCACAAUUACU | 3228 |
| | UUUGU | | UUUG | |
| miR-548o-3p | CCAAAACUGCAGUUACU | 3229 | GCAAAAGUAACUGCAGUU | 3230 |
| | UUUGC | | UUGG | |
| miR-548o-5p | AAAAGUAAUUGCGGUUU | 3231 | GGCAAAAACCGCAAUUAC | 3232 |
| | UUGCC | | UUUU | |
| miR-548p | UAGCAAAAACUGCAGUU | 3233 | AAAGUAACUGCAGUUUU | 3234 |
| | ACUUU | | UGCUA | |
| miR-548q | GCUGGUGCAAAAGUAAU | 3235 | CCGCCAUUACUUUUGCAC | 3236 |
| | GGCGG | | CAGC | |
| miR-548s | AUGGCCAAAACUGCAGU | 3237 | AAAAUAACUGCAGUUUU | 3238 |
| | UAUUUU | | GGCCAU | |
| miR-548t-3p | AAAAACCACAAUUACUU | 3239 | UGGUGCAAAAGUAAUUG | 3240 |
| | UUGCACCA | | UGGUUUUU | |
| miR-548t-5p | CAAAAGUGAUCGUGGUU | 3241 | CAAAAACCACGAUCACUU | 3242 |
| | UUUG | | UUG | |
| miR-548u | CAAAGACUGCAAUUACU | 3243 | CGCAAAAGUAAUUGCAGU | 3244 |
| | UUUGCG | | CUUUG | |
| miR-548v | AGCUACAGUUACUUUUG | 3245 | UGGUGCAAAAGUAACUG | 3246 |
| | CACCA | | UAGCU | |
| miR-548w | AAAAGUAACUGCGGUUU | 3247 | AGGCAAAAACCGCAGUUA | 3248 |
| | UUGCCU | | CUUUU | |
| miR-548x-3p | UAAAAACUGCAAUUACU | 3249 | GAAAGUAAUUGCAGUUU | 3250 |
| | UUC | | UUA | |
| miR-548x-5p | UGCAAAAGUAAUUGCAG | 3251 | CAAAAACUGCAAUUACUU | 3252 |
| | UUUUUG | | UUGCA | |
| miR-548y | AAAAGUAAUCACUGUUU | 3253 | GGCAAAAACAGUGAUUAC | 3254 |
| | UUGCC | | UUUU | |
| miR-548z | CAAAAACCGCAAUUACU | 3255 | UGCAAAAGUAAUUGCGG | 3256 |
| | UUUGCA | | UUUUUG | |
| miR-549 | UGACAACUAUGGAUGAG | 3257 | AGAGCUCAUCCAUAGUUG | 3258 |
| | CUCU | | UCA | |
| miR-550a-3-5p | AGUGCCUGAGGGAGUAA | 3259 | CUCUUACUCCCUCAGGCA | 3260 |
| | GAG | | CU | |
| miR-550a-3p | UGUCUUACUCCCUCAGG | 3261 | AUGUGCCUGAGGGAGUA | 3262 |
| | CACAU | | AGACA | |
| miR-550a-5p | AGUGCCUGAGGGAGUAA | 3263 | GGGCUCUUACUCCCUCAG | 3264 |
| | GAGCCC | | GCACU | |
| miR-550b-2-5p | AUGUGCCUGAGGGAGUA | 3265 | UGUCUUACUCCCUCAGGC | 3266 |
| | AGACA | | ACAU | |
| miR-550b-3p | UCUUACUCCCUCAGGCA | 3267 | CAGUGCCUGAGGGAGUAA | 3268 |
| | CUG | | GA | |
| miR-551a | GCGACCCACUCUUGGUU | 3269 | UGGAAACCAAGAGUGGG | 3270 |
| | UCCA | | UCGC | |
| miR-551b-3p | GCGACCCAUACUUGGUU | 3271 | CUGAAACCAAGUAUGGGU | 3272 |
| | UCAG | | CGC | |
| miR-551b-5p | GAAAUCAAGCGUGGGUG | 3273 | GGUCUCACCCACGCUUGA | 3274 |
| | AGACC | | UUUC | |
| miR-552 | AACAGGUGACUGGUUAG | 3275 | UUGUCUAACCAGUCACCU | 3276 |
| | ACAA | | GUU | |
| miR-553 | AAAACGGUGAGAUUUUG | 3277 | AAAACAAAAUCUCACCGU | 3278 |
| | UUUU | | UUU | |
| miR-554 | GCUAGUCCUGACUCAGC | 3279 | ACUGGCUGAGUCAGGACU | 3280 |
| | CAGU | | AGC | |
| miR-555 | AGGGUAAGCUGAACCUC | 3281 | AUCAGAGGUUCAGCUUAC | 3282 |
| | UGAU | | CCU | |
| miR-556-3p | AUAUUACCAUUAGCUCA | 3283 | AAAGAUGAGCUAAUGGU | 3284 |
| | UCUUU | | AAUAU | |
| miR-556-5p | GAUGAGCUCAUUGUAAU | 3285 | CUCAUAUUACAAUGAGCU | 3286 |
| | AUGAG | | CAUC | |
| miR-557 | GUUUGCACGGGUGGGCC | 3287 | AGACAAGGCCCACCCGUG | 3288 |
| | UUGUCU | | CAAAC | |
| miR-5571-3p | GUCCUAGGAGGCUCCUC | 3289 | CAGAGGAGCCUCCUAGGA | 3290 |
| | UG | | C | |
| miR-5571-5p | CAAUUCUCAAAGGAGCC | 3291 | GGGAGGCUCCUUUGAGAA | 3292 |
| | UCCC | | UUG | |
| miR-5572 | GUUGGGGUGCAGGGGUC | 3293 | AGCAGACCCCUGCACCCC | 3294 |
| | UGCU | | AAC | |
| miR-5579-3p | UUAGCUUAAGGAGUACC | 3295 | GAUCUGGUACUCCUUAAG | 3296 |
| | AGAUC | | CUAA | |
| miR-5579-5p | UAUGGUACUCCUUAAGC | 3297 | GUUAGCUUAAGGAGUACC | 3298 |
| | UAAC | | AUA | |
| miR-558 | UGAGCUGCUGUACCAAA | 3299 | AUUUUGGUACAGCAGCUC | 3300 |
| | AU | | A | |
| miR-5580-3p | CACAUAUGAAGUGAGCC | 3301 | GUGCUGGCUCACUUCAUA | 3302 |
| | AGCAC | | UGUG | |
| miR-5580-5p | UGCUGGCUCAUUUCAUA | 3303 | ACACAUAUGAAAUGAGCC | 3304 |
| | UGUGU | | AGCA | |
| miR-5581-3p | UUCCAUGCCUCCUAGAA | 3305 | GGAACUUCUAGGAGGCAU | 3306 |
| | GUUCC | | GGAA | |
| miR-5581-5p | AGCCUUCCAGGAGAAAU | 3307 | UCUCCAUUUCUCCUGGAA | 3308 |
| | GGAGA | | GGCU | |
| miR-5582-3p | UAAAACUUUAAGUGUGC | 3309 | CCUAGGCACACUUAAAGU | 3310 |
| | CUAGG | | UUUA | |
| miR-5582-5p | UAGGCACACUUAAAGUU | 3311 | GCUAUAACUUUAAGUGU | 3312 |
| | AUAGC | | GCCUA | |
| miR-5583-3p | GAAUAUGGGUAUAUUAG | 3313 | CCAAACUAAUAUACCCAU | 3314 |
| | UUUGG | | AUUC | |
| miR-5583-5p | AAACUAAUAUACCCAUA | 3315 | CAGAAUAUGGGUAUAUU | 3316 |
| | UUCUG | | AGUUU | |
| miR-5584-3p | UAGUUCUUCCCUUUGCC | 3317 | AAUUGGGCAAAGGGAAG | 3318 |
| | CAAUU | | AACUA | |
| miR-5584-5p | CAGGGAAAUGGGAAGAA | 3319 | UCUAGUUCUUCCCAUUUC | 3320 |
| | CUAGA | | CCUG | |
| miR-5585-3p | CUGAAUAGCUGGGACUA | 3321 | ACCUGUAGUCCCAGCUAU | 3322 |
| | CAGGU | | UCAG | |
| miR-5585-5p | UGAAGUACCAGCUACUC | 3323 | CUCUCGAGUAGCUGGUAC | 3324 |
| | GAGAG | | UUCA | |
| miR-5586-3p | CAGAGUGACAAGCUGGU | 3325 | CUUUAACCAGCUUGUCAC | 3326 |
| | UAAAG | | UCUG | |
| miR-5586-5p | UAUCCAGCUUGUUACUA | 3327 | GCAUAUAGUAACAAGCUG | 3328 |
| | UAUGC | | GAUA | |
| miR-5587-3p | GCCCCGGGCAGUGUGAU | 3329 | GAUGAUCACACUGCCCGG | 3330 |
| | CAUC | | GGC | |
| miR-5587-5p | AUGGUCACCUCCGGGAC | 3331 | AGUCCCGGAGGUGACCAU | 3332 |
| | U | | | |
| miR-5588-3p | AAGUCCCACUAAUGCCA | 3333 | GCUGGCAUUAGUGGGACU | 3334 |
| | GC | | U | |
| miR-5588-5p | ACUGGCAUUAGUGGGAC | 3335 | AAAAGUCCCACUAAUGCC | 3336 |
| | UUUU | | AGU | |
| miR-5589-3p | UGCACAUGGCAACCUAG | 3337 | UGGGAGCUAGGUUGCCAU | 3338 |
| | CUCCCA | | GUGCA | |
| miR-5589-5p | GGCUGGGUGCUCUUGUG | 3339 | ACUGCACAAGAGCACCCA | 3340 |
| | CAGU | | GCC | |
| miR-559 | UAAAGUAAAUAUGCACC | 3341 | UUUUGGUGCAUAUUUAC | 3342 |
| | AAAA | | UUUA | |
| miR-5590-3p | AAUAAAGUUCAUGUAUG | 3343 | UUGCCAUACAUGAACUUU | 3344 |
| | GCAA | | AUU | |
| miR-5590-5p | UUGCCAUACAUAGACUU | 3345 | AAUAAAGUCUAUGUAUG | 3346 |
| | UAUU | | GCAA | |
| miR-5591-3p | AUACCCAUAGCUUAGCU | 3347 | UGGGAGCUAAGCUAUGG | 3348 |
| | CCCA | | GUAU | |
| miR-5591-5p | UGGGAGCUAAGCUAUGG | 3349 | AUACCCAUAGCUUAGCUC | 3350 |
| | GUAU | | CCA | |
| miR-561-3p | CAAAGUUUAAGAUCCUU | 3351 | ACUUCAAGGAUCUUAAAC | 3352 |
| | GAAGU | | UUUG | |
| miR-561-5p | AUCAAGGAUCUUAAACU | 3353 | GGCAAAGUUUAAGAUCCU | 3354 |
| | UUGCC | | UGAU | |
| miR-562 | AAAGUAGCUGUACCAUU | 3355 | GCAAAUGGUACAGCUACU | 3356 |
| | UGC | | UU | |
| miR-563 | AGGUUGACAUACGUUUC | 3357 | GGGAAACGUAUGUCAACC | 3358 |
| | CC | | U | |
| miR-564 | AGGCACGGUGUCAGCAG | 3359 | GCCUGCUGACACCGUGCC | 3360 |
| | GC | | U | |
| miR-566 | GGGCGCCUGUGAUCCCA | 3361 | GUUGGGAUCACAGGCGCC | 3362 |
| | AC | | C | |
| miR-567 | AGUAUGUUCUUCCAGGA | 3363 | GUUCUGUCCUGGAAGAAC | 3364 |
| | CAGAAC | | AUACU | |
| miR-568 | AUGUAUAAAUGUAUACA | 3365 | GUGUGUAUACAUUUAUA | 3366 |
| | CAC | | CAU | |
| miR-5680 | GAGAAAUGCUGGACUAA | 3367 | GCAGAUUAGUCCAGCAUU | 3368 |
| | UCUGC | | UCUC | |
| miR-5681a | AGAAAGGGUGGCAAUAC | 3369 | AAGAGGUAUUGCCACCCU | 3370 |
| | CUCUU | | UUCU | |
| miR-5681b | AGGUAUUGCCACCCUUU | 3371 | ACUAGAAAGGGUGGCAA | 3372 |
| | CUAGU | | UACCU | |
| miR-5682 | GUAGCACCUUGCAGGAU | 3373 | ACCUUAUCCUGCAAGGUG | 3374 |
| | AAGGU | | CUAC | |
| miR-5683 | UACAGAUGCAGAUUCUC | 3375 | GAAGUCAGAGAAUCUGCA | 3376 |
| | UGACUUC | | UCUGUA | |
| miR-5684 | AACUCUAGCCUGAGCAA | 3377 | CUGUUGCUCAGGCUAGAG | 3378 |
| | CAG | | UU | |
| miR-5685 | ACAGCCCAGCAGUUAUC | 3379 | CCCGUGAUAACUGCUGGG | 3380 |
| | ACGGG | | CUGU | |
| miR-5686 | UAUCGUAUCGUAUUGUA | 3381 | ACAAUACAAUACGAUACG | 3382 |
| | UUGU | | AUA | |
| miR-5687 | UUAGAACGUUUUAGGGU | 3383 | AUUUGACCCUAAAACGUU | 3384 |
| | CAAAU | | CUAA | |
| miR-5688 | UAACAAACACCUGUAAA | 3385 | GCUGUUUUACAGGUGUU | 3386 |
| | ACAGC | | UGUUA | |
| miR-5689 | AGCAUACACCUGUAGUC | 3387 | UCUAGGACUACAGGUGUA | 3388 |
| | CUAGA | | UGCU | |
| miR-569 | AGUUAAUGAAUCCUGGA | 3389 | ACUUUCCAGGAUUCAUUA | 3390 |
| | AAGU | | ACU | |
| miR-5690 | UCAGCUACUACCUCUAU | 3391 | CCUAAUAGAGGUAGUAGC | 3392 |
| | UAGG | | UGA | |
| miR-5691 | UUGCUCUGAGCUCCGAG | 3393 | GCUUUCUCGGAGCUCAGA | 3394 |
| | AAAGC | | GCAA | |
| miR-5692a | CAAAUAAUACCACAGUG | 3395 | ACACCCACUGUGGUAUUA | 3396 |
| | GGUGU | | UUUG | |
| miR-5692b | AAUAAUAUCACAGUAGG | 3397 | ACACCUACUGUGAUAUUA | 3398 |
| | UGU | | UU | |
| miR-5692c | AAUAAUAUCACAGUAGG | 3399 | GUACACCUACUGUGAUAU | 3400 |
| | UGUAC | | UAUU | |
| miR-5693 | GCAGUGGCUCUGAAAUG | 3401 | GAGUUCAUUUCAGAGCCA | 3402 |
| | AACUC | | CUGC | |
| miR-5694 | CAGAUCAUGGGACUGUC | 3403 | CUGAGACAGUCCCAUGAU | 3404 |
| | UCAG | | CUG | |
| miR-5695 | ACUCCAAGAAGAAUCUA | 3405 | CUGUCUAGAUUCUUCUUG | 3406 |
| | GACAG | | GAGU | |
| miR-5696 | CUCAUUUAAGUAGUCUG | 3407 | GGCAUCAGACUACUUAAA | 3408 |
| | AUGCC | | UGAG | |
| miR-5697 | UCAAGUAGUUUCAUGAU | 3409 | CCUUUAUCAUGAAACUAC | 3410 |
| | AAAGG | | UUGA | |
| miR-5698 | UGGGGGAGUGCAGUGAU | 3411 | CCACAAUCACUGCACUCC | 3412 |
| | UGUGG | | CCCA | |
| miR-5699 | UCCUGUCUUUCCUUGUU | 3413 | GCUCCAACAAGGAAAGAC | 3414 |
| | GGAGC | | AGGA | |
| miR-570-3p | CGAAAACAGCAAUUACC | 3415 | GCAAAGGUAAUUGCUGU | 3416 |
| | UUUGC | | UUUCG | |
| miR-570-5p | AAAGGUAAUUGCAGUUU | 3417 | GGGAAAAACUGCAAUUAC | 3418 |
| | UUCCC | | CUUU | |
| miR-5700 | UAAUGCAUUAAAUUAUU | 3419 | CCUUCAAUAAUUUAAUGC | 3420 |
| | GAAGG | | AUUA | |
| miR-5701 | UUAUUGUCACGUUCUGA | 3421 | AAUCAGAACGUGACAAUA | 3422 |
| | UU | | A | |
| miR-5702 | UGAGUCAGCAACAUAUC | 3423 | CAUGGGAUAUGUUGCUG | 3424 |
| | CCAUG | | ACUCA | |
| miR-5703 | AGGAGAAGUCGGGAAGG | 3425 | ACCUUCCCGACUUCUCCU | 3426 |
| | U | | | |
| miR-5704 | UUAGGCCAUCAUCCCAU | 3427 | GCAUAAUGGGAUGAUGG | 3428 |
| | UAUGC | | CCUAA | |
| miR-5705 | UGUUUCGGGGCUCAUGG | 3429 | CACAGGCCAUGAGCCCCG | 3430 |
| | CCUGUG | | AAACA | |
| miR-5706 | UUCUGGAUAACAUGCUG | 3431 | AGCUUCAGCAUGUUAUCC | 3432 |
| | AAGCU | | AGAA | |
| miR-5707 | ACGUUUGAAUGCUGUAC | 3433 | GCCUUGUACAGCAUUCAA | 3434 |
| | AAGGC | | ACGU | |
| miR-5708 | AUGAGCGACUGUGCCUG | 3435 | GGUCAGGCACAGUCGCUC | 3436 |
| | ACC | | AU | |
| miR-571 | UGAGUUGGCCAUCUGAG | 3437 | CUCACUCAGAUGGCCAAC | 3438 |
| | UGAG | | UCA | |
| miR-572 | GUCCGCUCGGCGGUGGC | 3439 | UGGGCCACCGCCGAGCGG | 3440 |
| | CCA | | AC | |
| miR-573 | CUGAAGUGAUGUGUAAC | 3441 | CUGAUCAGUUACACAUCA | 3442 |
| | UGAUCAG | | CUUCAG | |
| miR-574-3p | CACGCUCAUGCACACAC | 3443 | UGUGGGUGUGUGCAUGA | 3444 |
| | CCACA | | GCGUG | |
| miR-574-5p | UGAGUGUGUGUGUGUGA | 3445 | ACACACUCACACACACAC | 3446 |
| | GUGUGU | | ACUCA | |
| miR-575 | GAGCCAGUUGGACAGGA | 3447 | GCUCCUGUCCAACUGGCU | 3448 |
| | GC | | C | |
| miR-576-3p | AAGAUGUGGAAAAAUUG | 3449 | GAUUCCAAUUUUUCCACA | 3450 |
| | GAAUC | | UCUU | |
| miR-576-5p | AUUCUAAUUUCUCCACG | 3451 | AAAGACGUGGAGAAAUU | 3452 |
| | UCUUU | | AGAAU | |
| miR-577 | UAGAUAAAAUAUUGGUA | 3453 | CAGGUACCAAUAUUUUAU | 3454 |
| | CCUG | | CUA | |
| miR-578 | CUUCUUGUGCUCUAGGA | 3455 | ACAAUCCUAGAGCACAAG | 3456 |
| | UUGU | | AAG | |
| miR-579 | UUCAUUUGGUAUAAACC | 3457 | AAUCGCGGUUUAUACCAA | 3458 |
| | GCGAUU | | AUGAA | |
| miR-580 | UUGAGAAUGAUGAAUCA | 3459 | CCUAAUGAUUCAUCAUUC | 3460 |
| | UUAGG | | UCAA | |
| miR-581 | UCUUGUGUUCUCUAGAU | 3461 | ACUGAUCUAGAGAACACA | 3462 |
| | CAGU | | AGA | |
| miR-582-3p | UAACUGGUUGAACAACU | 3463 | GGUUCAGUUGUUCAACCA | 3464 |
| | GAACC | | GUUA | |
| miR-582-5p | UUACAGUUGUUCAACCA | 3465 | AGUAACUGGUUGAACAAC | 3466 |
| | GUUACU | | UGUAA | |
| miR-583 | CAAAGAGGAAGGUCCCA | 3467 | GUAAUGGGACCUUCCUCU | 3468 |
| | UUAC | | UUG | |
| miR-584-3p | UCAGUUCCAGGCCAACC | 3469 | AGCCUGGUUGGCCUGGAA | 3470 |
| | AGGCU | | CUGA | |
| miR-584-5p | UUAUGGUUUGCCUGGGA | 3471 | CUCAGUCCCAGGCAAACC | 3472 |
| | CUGAG | | AUAA | |
| miR-585 | UGGGCGUAUCUGUAUGC | 3473 | UAGCAUACAGAUACGCCC | 3474 |
| | UA | | A | |
| miR-586 | UAUGCAUUGUAUUUUUA | 3475 | GGACCUAAAAAUACAAUG | 3476 |
| | GGUCC | | CAUA | |
| miR-587 | UUUCCAUAGGUGAUGAG | 3477 | GUGACUCAUCACCUAUGG | 3478 |
| | UCAC | | AAA | |
| miR-588 | UUGGCCACAAUGGGUUA | 3479 | GUUCUAACCCAUUGUGGC | 3480 |
| | GAAC | | CAA | |
| miR-589-3p | UCAGAACAAAUGCCGGU | 3481 | UCUGGGAACCGGCAUUUG | 3482 |
| | UCCCAGA | | UUCUGA | |
| miR-589-5p | UGAGAACCACGUCUGCU | 3483 | CUCAGAGCAGACGUGGUU | 3484 |
| | CUGAG | | CUCA | |
| miR-590-3p | UAAUUUUAUGUAUAAGC | 3485 | ACUAGCUUAUACAUAAAA | 3486 |
| | UAGU | | UUA | |
| miR-590-5p | GAGCUUAUUCAUAAAAG | 3487 | CUGCACUUUUAUGAAUAA | 3488 |
| | UGCAG | | GCUC | |
| miR-591 | AGACCAUGGGUUCUCAU | 3489 | ACAAUGAGAACCCAUGGU | 3490 |
| | UGU | | CU | |
| miR-592 | UUGUGUCAAUAUGCGAU | 3491 | ACAUCAUCGCAUAUUGAC | 3492 |
| | GAUGU | | ACAA | |
| miR-593-3p | UGUCUCUGCUGGGGUUU | 3493 | AGAAACCCCAGCAGAGAC | 3494 |
| | CU | | A | |
| miR-593-5p | AGGCACCAGCCAGGCAU | 3495 | GCUGAGCAAUGCCUGGCU | 3496 |
| | UGCUCAGC | | GGUGCCU | |
| miR-595 | GAAGUGUGCCGUGGUGU | 3497 | AGACACACCACGGCACAC | 3498 |
| | GUCU | | UUC | |
| miR-596 | AAGCCUGCCCGGCUCCU | 3499 | CCCGAGGAGCCGGGCAGG | 3500 |
| | CGGG | | CUU | |
| miR-597 | UGUGUCACUCGAUGACC | 3501 | ACAGUGGUCAUCGAGUGA | 3502 |
| | ACUGU | | CACA | |
| miR-598 | UACGUCAUCGUUGUCAU | 3503 | UGACGAUGACAACGAUGA | 3504 |
| | CGUCA | | CGUA | |
| miR-599 | GUUGUGUCAGUUUAUCA | 3505 | GUUUGAUAAACUGACACA | 3506 |
| | AAC | | AC | |
| miR-600 | ACUUACAGACAAGAGCC | 3507 | GAGCAAGGCUCUUGUCUG | 3508 |
| | UUGCUC | | UAAGU | |
| miR-601 | UGGUCUAGGAUUGUUGG | 3509 | CUCCUCCAACAAUCCUAG | 3510 |
| | AGGAG | | ACCA | |
| miR-602 | GACACGGGCGACAGCUG | 3511 | GGGCCGCAGCUGUCGCCC | 3512 |
| | CGGCCC | | GUGUC | |
| miR-603 | CACACACUGCAAUUACU | 3513 | GCAAAAGUAAUUGCAGU | 3514 |
| | UUUGC | | GUGUG | |
| miR-604 | AGGCUGCGGAAUUCAGG | 3515 | GUCCUGAAUUCCGCAGCC | 3516 |
| | AC | | U | |
| miR-605 | UAAAUCCCAUGGUGCCU | 3517 | AGGAGAAGGCACCAUGGG | 3518 |
| | UCUCCU | | AUUUA | |
| miR-606 | AAACUACUGAAAAUCAA | 3519 | AUCUUUGAUUUUCAGUA | 3520 |
| | AGAU | | GUUU | |
| miR-607 | GUUCAAAUCCAGAUCUA | 3521 | GUUAUAGAUCUGGAUUU | 3522 |
| | UAAC | | GAAC | |
| miR-608 | AGGGGUGGUGUUGGGAC | 3523 | ACGGAGCUGUCCCAACAC | 3524 |
| | AGCUCCGU | | CACCCCU | |
| miR-609 | AGGGUGUUUCUCUCAUC | 3525 | AGAGAUGAGAGAAACACC | 3526 |
| | UCU | | CU | |
| miR-610 | UGAGCUAAAUGUGUGCU | 3527 | UCCCAGCACACAUUUAGC | 3528 |
| | GGGA | | UCA | |
| miR-611 | GCGAGGACCCCUCGGGG | 3529 | GUCAGACCCCGAGGGGUC | 3530 |
| | UCUGAC | | CUCGC | |
| miR-612 | GCUGGGCAGGGCUUCUG | 3531 | AAGGAGCUCAGAAGCCCU | 3532 |
| | AGCUCCUU | | GCCCAGC | |
| miR-613 | AGGAAUGUUCCUUCUUU | 3533 | GGCAAAGAAGGAACAUUC | 3534 |
| | GCC | | CU | |
| miR-614 | GAACGCCUGUUCUUGCC | 3535 | CCACCUGGCAAGAACAGG | 3536 |
| | AGGUGG | | CGUUC | |
| miR-615-3p | UCCGAGCCUGGGUCUCC | 3537 | AAGAGGGAGACCCAGGCU | 3538 |
| | CUCUU | | CGGA | |
| miR-615-5p | GGGGGUCCCCGGUGCUC | 3539 | GAUCCGAGCACCGGGGAC | 3540 |
| | GGAUC | | CCCC | |
| miR-616-3p | AGUCAUUGGAGGGUUUG | 3541 | CUGCUCAAACCCUCCAAU | 3542 |
| | AGCAG | | GACU | |
| miR-616-5p | ACUCAAAACCCUUCAGU | 3543 | AAGUCACUGAAGGGUUU | 3544 |
| | GACUU | | UGAGU | |
| miR-617 | AGACUUCCCAUUUGAAG | 3545 | GCCACCUUCAAAUGGGAA | 3546 |
| | GUGGC | | GUCU | |
| miR-618 | AAACUCUACUUGUCCUU | 3547 | ACUCAGAAGGACAAGUAG | 3548 |
| | CUGAGU | | AGUUU | |
| miR-619 | GACCUGGACAUGUUUGU | 3549 | ACUGGGCACAAACAUGUC | 3550 |
| | GCCCAGU | | CAGGUC | |
| miR-620 | AUGGAGAUAGAUAUAGA | 3551 | AUUUCUAUAUCUAUCUCC | 3552 |
| | AAU | | AU | |
| miR-621 | GGCUAGCAACAGCGCUU | 3553 | AGGUAAGCGCUGUUGCUA | 3554 |
| | ACCU | | GCC | |
| miR-622 | ACAGUCUGCUGAGGUUG | 3555 | GCUCCAACCUCAGCAGAC | 3556 |
| | GAGC | | UGU | |
| miR-623 | AUCCCUUGCAGGGGCUG | 3557 | ACCCAACAGCCCCUGCAA | 3558 |
| | UUGGGU | | GGGAU | |
| miR-624-3p | CACAAGGUAUUGGUAUU | 3559 | AGGUAAUACCAAUACCUU | 3560 |
| | ACCU | | GUG | |
| miR-624-5p | UAGUACCAGUACCUUGU | 3561 | UGAACACAAGGUACUGGU | 3562 |
| | GUUCA | | ACUA | |
| miR-625-3p | GACUAUAGAACUUUCCC | 3563 | UGAGGGGGAAAGUUCUA | 3564 |
| | CCUCA | | UAGUC | |
| miR-625-5p | AGGGGGAAAGUUCUAUA | 3565 | GGACUAUAGAACUUUCCC | 3566 |
| | GUCC | | CCU | |
| miR-626 | AGCUGUCUGAAAAUGUC | 3567 | AAGACAUUUUCAGACAGC | 3568 |
| | UU | | U | |
| miR-627 | GUGAGUCUCUAAGAAAA | 3569 | UCCUCUUUUCUUAGAGAC | 3570 |
| | GAGGA | | UCAC | |
| miR-628-3p | UCUAGUAAGAGUGGCAG | 3571 | UCGACUGCCACUCUUACU | 3572 |
| | UCGA | | AGA | |
| miR-628-5p | AUGCUGACAUAUUUACU | 3573 | CCUCUAGUAAAUAUGUCA | 3574 |
| | AGAGG | | GCAU | |
| miR-629-3p | GUUCUCCCAACGUAAGC | 3575 | GCUGGGCUUACGUUGGGA | 3576 |
| | CCAGC | | GAAC | |
| miR-629-5p | UGGGUUUACGUUGGGAG | 3577 | AGUUCUCCCAACGUAAAC | 3578 |
| | AACU | | CCA | |
| miR-630 | AGUAUUCUGUACCAGGG | 3579 | ACCUUCCCUGGUACAGAA | 3580 |
| | AAGGU | | UACU | |
| miR-631 | AGACCUGGCCCAGACCU | 3581 | GCUGAGGUCUGGGCCAGG | 3582 |
| | CAGC | | UCU | |
| miR-632 | GUGUCUGCUUCCUGUGG | 3583 | UCCCACAGGAAGCAGACA | 3584 |
| | GA | | C | |
| miR-633 | CUAAUAGUAUCUACCAC | 3585 | UUUAUUGUGGUAGAUAC | 3586 |
| | AAUAAA | | UAUUAG | |
| miR-634 | AACCAGCACCCCAACUU | 3587 | GUCCAAAGUUGGGGUGCU | 3588 |
| | UGGAC | | GGUU | |
| miR-635 | ACUUGGGCACUGAAACA | 3589 | GGACAUUGUUUCAGUGCC | 3590 |
| | AUGUCC | | CAAGU | |
| miR-636 | UGUGCUUGCUCGUCCCG | 3591 | UGCGGGCGGGACGAGCAA | 3592 |
| | CCCGCA | | GCACA | |
| miR-637 | ACUGGGGGCUUUCGGGC | 3593 | ACGCAGAGCCCGAAAGCC | 3594 |
| | UCUGCGU | | CCCAGU | |
| miR-638 | AGGGAUCGCGGGCGGGU | 3595 | AGGCCGCCACCCGCCCGC | 3596 |
| | GGCGGCCU | | GAUCCCU | |
| miR-639 | AUCGCUGCGGUUGCGAG | 3597 | ACAGCGCUCGCAACCGCA | 3598 |
| | CGCUGU | | GCGAU | |
| miR-640 | AUGAUCCAGGAACCUGC | 3599 | AGAGGCAGGUUCCUGGAU | 3600 |
| | CUCU | | CAU | |
| miR-641 | AAAGACAUAGGAUAGAG | 3601 | GAGGUGACUCUAUCCUAU | 3602 |
| | UCACCUC | | GUCUUU | |
| miR-642a-3p | AGACACAUUUGGAGAGG | 3603 | GGUUCCCUCUCCAAAUGU | 3604 |
| | GAACC | | GUCU | |
| miR-642a-5p | GUCCCUCUCCAAAUGUG | 3605 | CAAGACACAUUUGGAGAG | 3606 |
| | UCUUG | | GGAC | |
| miR-642b-3p | AGACACAUUUGGAGAGG | 3607 | GGGUCCCUCUCCAAAUGU | 3608 |
| | GACCC | | GUCU | |
| miR-642b-5p | GGUUCCCUCUCCAAAUG | 3609 | AGACACAUUUGGAGAGG | 3610 |
| | UGUCU | | GAACC | |
| miR-643 | ACUUGUAUGCUAGCUCA | 3611 | CUACCUGAGCUAGCAUAC | 3612 |
| | GGUAG | | AAGU | |
| miR-644a | AGUGUGGCUUUCUUAGA | 3613 | GCUCUAAGAAAGCCACAC | 3614 |
| | GC | | U | |
| miR-644b-3p | UUCAUUUGCCUCCCAGC | 3615 | UGUAGGCUGGGAGGCAA | 3616 |
| | CUACA | | AUGAA | |
| miR-644b-5p | UGGGCUAAGGGAGAUGA | 3617 | UACCCAAUCAUCUCCCUU | 3618 |
| | UUGGGUA | | AGCCCA | |
| miR-645 | UCUAGGCUGGUACUGCU | 3619 | UCAGCAGUACCAGCCUAG | 3620 |
| | GA | | A | |
| miR-646 | AAGCAGCUGCCUCUGAG | 3621 | GCCUCAGAGGCAGCUGCU | 3622 |
| | GC | | U | |
| miR-647 | GUGGCUGCACUCACUUC | 3623 | GAAGGAAGUGAGUGCAG | 3624 |
| | CUUC | | CCAC | |
| miR-648 | AAGUGUGCAGGGCACUG | 3625 | ACCAGUGCCCUGCACACU | 3626 |
| | GU | | U | |
| miR-649 | AAACCUGUGUUGUUCAA | 3627 | GACUCUUGAACAACACAG | 3628 |
| | GAGUC | | GUUU | |
| miR-650 | AGGAGGCAGCGCUCUCA | 3629 | GUCCUGAGAGCGCUGCCU | 3630 |
| | GGAC | | CCU | |
| miR-651 | UUUAGGAUAAGCUUGAC | 3631 | CAAAAGUCAAGCUUAUCC | 3632 |
| | UUUUG | | UAAA | |
| miR-652-3p | AAUGGCGCCACUAGGGU | 3633 | CACAACCCUAGUGGCGCC | 3634 |
| | UGUG | | AUU | |
| miR-652-5p | CAACCCUAGGAGAGGGU | 3635 | UGAAUGGCACCCUCUCCU | 3636 |
| | GCCAUUCA | | AGGGUUG | |
| miR-653 | GUGUUGAAACAAUCUCU | 3637 | CAGUAGAGAUUGUUUCA | 3638 |
| | ACUG | | ACAC | |
| miR-654-3p | UAUGUCUGCUGACCAUC | 3639 | AAGGUGAUGGUCAGCAG | 3640 |
| | ACCUU | | ACAUA | |
| miR-654-5p | UGGUGGGCCGCAGAACA | 3641 | GCACAUGUUCUGCGGCCC | 3642 |
| | UGUGC | | ACCA | |
| miR-655 | AUAAUACAUGGUUAACC | 3643 | AAAGAGGUUAACCAUGU | 3644 |
| | UCUUU | | AUUAU | |
| miR-656 | AAUAUUAUACAGUCAAC | 3645 | AGAGGUUGACUGUAUAA | 3646 |
| | CUCU | | UAUU | |
| miR-657 | GGCAGGUUCUCACCCUC | 3647 | CCUAGAGAGGGUGAGAAC | 3648 |
| | UCUAGG | | CUGCC | |
| miR-658 | GGCGGAGGGAAGUAGGU | 3649 | ACCAACGGACCUACUUCC | 3650 |
| | CCGUUGGU | | CUCCGCC | |
| miR-659-3p | CUUGGUUCAGGGAGGGU | 3651 | UGGGGACCCUCCCUGAAC | 3652 |
| | CCCCA | | CAAG | |
| miR-659-5p | AGGACCUUCCCUGAACC | 3653 | UCCUUGGUUCAGGGAAGG | 3654 |
| | AAGGA | | UCCU | |
| miR-660-3p | ACCUCCUGUGUGCAUGG | 3655 | UAAUCCAUGCACACAGGA | 3656 |
| | AUUA | | GGU | |
| miR-660-5p | UACCCAUUGCAUAUCGG | 3657 | CAACUCCGAUAUGCAAUG | 3658 |
| | AGUUG | | GGUA | |
| miR-661 | UGCCUGGGUCUCUGGCC | 3659 | ACGCGCAGGCCAGAGACC | 3660 |
| | UGCGCGU | | CAGGCA | |
| miR-662 | UCCCACGUUGUGGCCCA | 3661 | CUGCUGGGCCACAACGUG | 3662 |
| | GCAG | | GGA | |
| miR-663a | AGGCGGGGCGCCGCGGG | 3663 | GCGGTCCCGCGGCGCCCC | 3664 |
| | ACCGC | | GCCT | |
| miR-663b | GGUGGCCCGGCCGUGCC | 3665 | CCUCAGGCACGGCCGGGC | 3666 |
| | UGAGG | | CACC | |
| miR-664-3p | UAUUCAUUUAUCCCCAG | 3667 | UGUAGGCUGGGGAUAAA | 3668 |
| | CCUACA | | UGAAUA | |
| miR-664-5p | ACUGGCUAGGGAAAAUG | 3669 | AUCCAAUCAUUUUCCCUA | 3670 |
| | AUUGGAU | | GCCAGU | |
| miR-665 | ACCAGGAGGCUGAGGCC | 3671 | AGGGGCCUCAGCCUCCUG | 3672 |
| | CCU | | GU | |
| miR-668 | UGUCACUCGGCUCGGCC | 3673 | GUAGUGGGCCGAGCCGAG | 3674 |
| | CACUAC | | UGACA | |
| miR-670 | GUCCCUGAGUGUAUGUG | 3675 | CACCACAUACACUCAGGG | 3676 |
| | GUG | | AC | |
| miR-671-3p | UCCGGUUCUCAGGGCUC | 3677 | GGUGGAGCCCUGAGAACC | 3678 |
| | CACC | | GGA | |
| miR-671-5p | AGGAAGCCCUGGAGGGG | 3679 | CUCCAGCCCCUCCAGGGC | 3680 |
| | CUGGAG | | UUCCU | |
| miR-675-3p | CUGUAUGCCCUCACCGC | 3681 | UGAGCGGUGAGGGCAUAC | 3682 |
| | UCA | | AG | |
| miR-675-5p | UGGUGCGGAGAGGGCCC | 3683 | CACUGUGGGCCCUCUCCG | 3684 |
| | ACAGUG | | CACCA | |
| miR-676-3p | CUGUCCUAAGGUUGUUG | 3685 | AACUCAACAACCUUAGGA | 3686 |
| | AGUU | | CAG | |
| miR-676-5p | UCUUCAACCUCAGGACU | 3687 | UGCAAGUCCUGAGGUUGA | 3688 |
| | UGCA | | AGA | |
| miR-7-1-3p | CAACAAAUCACAGUCUG | 3689 | UAUGGCAGACUGUGAUU | 3690 |
| | CCAUA | | UGUUG | |
| miR-7-2-3p | CAACAAAUCCCAGUCUA | 3691 | UUAGGUAGACUGGGAUU | 3692 |
| | CCUAA | | UGUUG | |
| miR-7-5p | UGGAAGACUAGUGAUUU | 3693 | ACAACAAAAUCACUAGUC | 3694 |
| | UGUUGU | | UUCCA | |
| miR-708-3p | CAACUAGACUGUGAGCU | 3695 | CUAGAAGCUCACAGUCUA | 3696 |
| | UCUAG | | GUUG | |
| miR-708-5p | AAGGAGCUUACAAUCUA | 3697 | CCCAGCUAGAUUGUAAGC | 3698 |
| | GCUGGG | | UCCUU | |
| miR-711 | GGGACCCAGGGAGAGAC | 3699 | CUUACGUCUCUCCCUGGG | 3700 |
| | GUAAG | | UCCC | |
| miR-718 | CUUCCGCCCCGCCGGGC | 3701 | CGACGCCCGGCGGGGCGG | 3702 |
| | GUCG | | AAG | |
| miR-720 | UCUCGCUGGGGCCUCCA | 3703 | UGGAGGCCCCAGCGAGA | 3704 |
| miR-744-3p | CUGUUGCCACUAACCUC | 3705 | AGGUUGAGGUUAGUGGC | 3706 |
| | AACCU | | AACAG | |
| miR-744-5p | UGCGGGGCUAGGGCUAA | 3707 | UGCUGUUAGCCCUAGCCC | 3708 |
| | CAGCA | | CGCA | |
| miR-758 | UUUGUGACCUGGUCCAC | 3709 | GGUUAGUGGACCAGGUCA | 3710 |
| | UAACC | | CAAA | |
| miR-759 | GCAGAGUGCAAACAAUU | 3711 | GUCAAAAUUGUUUGCACU | 3712 |
| | UUGAC | | CUGC | |
| miR-760 | CGGCUCUGGGUCUGUGG | 3713 | UCCCCACAGACCCAGAGC | 3714 |
| | GGA | | CG | |
| miR-761 | GCAGCAGGGUGAAACUG | 3715 | UGUGUCAGUUUCACCCUG | 3716 |
| | ACACA | | CUGC | |
| miR-762 | GGGGCUGGGGCCGGGGC | 3717 | GCUCGGCCCCGGCCCCAG | 3718 |
| | CGAGC | | CCCC | |
| miR-764 | GCAGGUGCUCACUUGUC | 3719 | AGGAGGACAAGUGAGCAC | 3720 |
| | CUCCU | | CUGC | |
| miR-765 | UGGAGGAGAAGGAAGGU | 3721 | CAUCACCUUCCUUCUCCU | 3722 |
| | GAUG | | CCA | |
| miR-766-3p | ACUCCAGCCCCACAGCC | 3723 | GCUGAGGCUGUGGGGCUG | 3724 |
| | UCAGC | | GAGU | |
| miR-766-5p | AGGAGGAAUUGGUGCUG | 3725 | AAGACCAGCACCAAUUCC | 3726 |
| | GUCUU | | UCCU | |
| miR-767-3p | UCUGCUCAUACCCCAUG | 3727 | AGAAACCAUGGGGUAUG | 3728 |
| | GUUUCU | | AGCAGA | |
| miR-767-5p | UGCACCAUGGUUGUCUG | 3729 | CAUGCUCAGACAACCAUG | 3730 |
| | AGCAUG | | GUGCA | |
| miR-769-3p | CUGGGAUCUCCGGGGUC | 3731 | AACCAAGACCCCGGAGAU | 3732 |
| | UUGGUU | | CCCAG | |
| miR-769-5p | UGAGACCUCUGGGUUCU | 3733 | AGCUCAGAACCCAGAGGU | 3734 |
| | GAGCU | | CUCA | |
| miR-770-5p | UCCAGUACCACGUGUCA | 3735 | UGGCCCUGACACGUGGUA | 3736 |
| | GGGCCA | | CUGGA | |
| miR-802 | CAGUAACAAAGAUUCAU | 3737 | ACAAGGAUGAAUCUUUG | 3738 |
| | CCUUGU | | UUACUG | |
| miR-873-3p | GGAGACUGAUGAGUUCC | 3739 | UCCCGGGAACUCAUCAGU | 3740 |
| | CGGGA | | CUCC | |
| miR-873-5p | GCAGGAACUUGUGAGUC | 3741 | AGGAGACUCACAAGUUCC | 3742 |
| | UCCU | | UGC | |
| miR-874 | CUGCCCUGGCCCGAGGG | 3743 | UCGGUCCCUCGGGCCAGG | 3744 |
| | ACCGA | | GCAG | |
| miR-875-3p | CCUGGAAACACUGAGGU | 3745 | CACAACCUCAGUGUUUCC | 3746 |
| | UGUG | | AGG | |
| miR-875-5p | UAUACCUCAGUUUUAUC | 3747 | CACCUGAUAAAACUGAGG | 3748 |
| | AGGUG | | UAUA | |
| miR-876-3p | UGGUGGUUUACAAAGUA | 3749 | UGAAUUACUUUGUAAACC | 3750 |
| | AUUCA | | ACCA | |
| miR-876-5p | UGGAUUUCUUUGUGAAU | 3751 | UGGUGAUUCACAAAGAA | 3752 |
| | CACCA | | AUCCA | |
| miR-877-3p | UCCUCUUCUCCCUCCUCC | 3753 | CUGGGAGGAGGGAGAAG | 3754 |
| | CAG | | AGGA | |
| miR-877-5p | GUAGAGGAGAUGGCGCA | 3755 | CCCUGCGCCAUCUCCUCU | 3756 |
| | GGG | | AC | |
| miR-885-3p | AGGCAGCGGGGUGUAGU | 3757 | UAUCCACUACACCCCGCU | 3758 |
| | GGAUA | | GCCU | |
| miR-885-5p | UCCAUUACACUACCCUG | 3759 | AGAGGCAGGGUAGUGUA | 3760 |
| | CCUCU | | AUGGA | |
| miR-887 | GUGAACGGGCGCCAUCC | 3761 | CCUCGGGAUGGCGCCCGU | 3762 |
| | CGAGG | | UCAC | |
| miR-888-3p | GACUGACACCUCUUUGG | 3763 | UUCACCCAAAGAGGUGUC | 3764 |
| | GUGAA | | AGUC | |
| miR-888-5p | UACUCAAAAAGCUGUCA | 3765 | UGACUGACAGCUUUUUGA | 3766 |
| | GUCA | | GUA | |
| miR-889 | UUAAUAUCGGACAACCA | 3767 | ACAAUGGUUGUCCGAUAU | 3768 |
| | UUGU | | UAA | |
| miR-890 | UACUUGGAAAGGCAUCA | 3769 | CAACUGAUGCCUUUCCAA | 3770 |
| | GUUG | | GUA | |
| miR-891a | UGCAACGAACCUGAGCC | 3771 | UCAGUGGCUCAGGUUCGU | 3772 |
| | ACUGA | | UGCA | |
| miR-891b | UGCAACUUACCUGAGUC | 3773 | UCAAUGACUCAGGUAAGU | 3774 |
| | AUUGA | | UGCA | |
| miR-892a | CACUGUGUCCUUUCUGC | 3775 | CUACGCAGAAAGGACACA | 3776 |
| | GUAG | | GUG | |
| miR-892b | CACUGGCUCCUUUCUGG | 3777 | UCUACCCAGAAAGGAGCC | 3778 |
| | GUAGA | | AGUG | |
| miR-9-3p | AUAAAGCUAGAUAACCG | 3779 | ACUUUCGGUUAUCUAGCU | 3780 |
| | AAAGU | | UUAU | |
| miR-9-5p | UCUUUGGUUAUCUAGCU | 3781 | UCAUACAGCUAGAUAACC | 3782 |
| | GUAUGA | | AAAGA | |
| miR-920 | GGGGAGCUGUGGAAGCA | 3783 | UACUGCUUCCACAGCUCC | 3784 |
| | GUA | | CC | |
| miR-921 | CUAGUGAGGGACAGAAC | 3785 | GAAUCCUGGUUCUGUCCC | 3786 |
| | CAGGAUUC | | UCACUAG | |
| miR-922 | GCAGCAGAGAAUAGGAC | 3787 | GACGUAGUCCUAUUCUCU | 3788 |
| | UACGUC | | GCUGC | |
| miR-924 | AGAGUCUUGUGAUGUCU | 3789 | GCAAGACAUCACAAGACU | 3790 |
| | UGC | | CU | |
| miR-92a-1-5p | AGGUUGGGAUCGGUUGC | 3791 | AGCAUUGCAACCGAUCCC | 3792 |
| | AAUGCU | | AACCU | |
| miR-92a-2-5p | GGGUGGGGAUUUGUUGC | 3793 | GUAAUGCAACAAAUCCCC | 3794 |
| | AUUAC | | ACCC | |
| miR-92a-3p | UAUUGCACUUGUCCCGG | 3795 | ACAGGCCGGGACAAGUGC | 3796 |
| | CCUGU | | AAUA | |
| miR-92b-3p | UAUUGCACUCGUCCCGG | 3797 | GGAGGCCGGGACGAGUGC | 3798 |
| | CCUCC | | AAUA | |
| miR-92b-5p | AGGGACGGGACGCGGUG | 3799 | CACUGCACCGCGUCCCGU | 3800 |
| | CAGUG | | CCCU | |
| miR-93-3p | ACUGCUGAGCUAGCACU | 3801 | CGGGAAGUGCUAGCUCAG | 3802 |
| | UCCCG | | CAGU | |
| miR-93-5p | CAAAGUGCUGUUCGUGC | 3803 | CUACCUGCACGAACAGCA | 3804 |
| | AGGUAG | | CUUUG | |
| miR-933 | UGUGCGCAGGGAGACCU | 3805 | GGGAGAGGUCUCCCUGCG | 3806 |
| | CUCCC | | CACA | |
| miR-934 | UGUCUACUACUGGAGAC | 3807 | CCAGUGUCUCCAGUAGUA | 3808 |
| | ACUGG | | GACA | |
| miR-935 | CCAGUUACCGCUUCCGC | 3809 | GCGGUAGCGGAAGCGGUA | 3810 |
| | UACCGC | | ACUGG | |
| miR-936 | ACAGUAGAGGGAGGAAU | 3811 | CUGCGAUUCCUCCCUCUA | 3812 |
| | CGCAG | | CUGU | |
| miR-937 | AUCCGCGCUCUGACUCU | 3813 | GGCAGAGAGUCAGAGCGC | 3814 |
| | CUGCC | | GGAU | |
| miR-938 | UGCCCUUAAAGGUGAAC | 3815 | ACUGGGUUCACCUUUAAG | 3816 |
| | CCAGU | | GGCA | |
| miR-939 | UGGGGAGCUGAGGCUCU | 3817 | CACCCCCAGAGCCUCAGC | 3818 |
| | GGGGGUG | | UCCCCA | |
| miR-940 | AAGGCAGGGCCCCCGCU | 3819 | GGGGAGCGGGGGCCCUGC | 3820 |
| | CCCC | | CUU | |
| miR-941 | CACCCGGCUGUGUGCAC | 3821 | GCACAUGUGCACACAGCC | 3822 |
| | AUGUGC | | GGGUG | |
| miR-942 | UCUUCUCUGUUUUGGCC | 3823 | CACAUGGCCAAAACAGAG | 3824 |
| | AUGUG | | AAGA | |
| miR-943 | CUGACUGUUGCCGUCCU | 3825 | CUGGAGGACGGCAACAGU | 3826 |
| | CCAG | | CAG | |
| miR-944 | AAAUUAUUGUACAUCGG | 3827 | CUCAUCCGAUGUACAAUA | 3828 |
| | AUGAG | | AUUU | |
| miR-95 | UUCAACGGGUAUUUAUU | 3829 | UGCUCAAUAAAUACCCGU | 3830 |
| | GAGCA | | UGAA | |
| miR-96-3p | AAUCAUGUGCAGUGCCA | 3831 | CAUAUUGGCACUGCACAU | 3832 |
| | AUAUG | | GAUU | |
| miR-96-5p | UUUGGCACUAGCACAUU | 3833 | AGCAAAAAUGUGCUAGU | 3834 |
| | UUUGCU | | GCCAAA | |
| miR-98 | UGAGGUAGUAAGUUGUA | 3835 | AACAAUACAACUUACUAC | 3836 |
| | UUGUU | | CUCA | |
| miR-99a-3p | CAAGCUCGCUUCUAUGG | 3837 | CAGACCCAUAGAAGCGAG | 3838 |
| | GUCUG | | CUUG | |
| miR-99a-5p | AACCCGUAGAUCCGAUC | 3839 | CACAAGAUCGGAUCUACG | 3840 |
| | UUGUG | | GGUU | |
| miR-99b-3p | CAAGCUCGUGUCUGUGG | 3841 | CGGACCCACAGACACGAG | 3842 |
| | GUCCG | | CUUG | |
| miR-99b-5p | CACCCGUAGAACCGACC | 3843 | CGCAAGGUCGGUUCUACG | 3844 |
| | UUGCG | | GGUG | |

In some embodiments, miRNA seeds, which may be incorporated into viral target sequences to create a miRNA binding site are 2-8 nucleobases in length. One having ordinary skill in the art will appreciate that this embodies compounds of 2, 3, 4, 5, 6, 7 or 8 nucleobases in length, or any range therewithin.

miRNA binding sites may be engineered into a viral sequence based on tissue specificity. For example, sites may be created to encourage or facilitate the binding of miRNA found in neuronal cells or epithelial cells. Table 4 lists the sequence of miRNA found to be expressed in the brain. Sequences which comprise all or a portion of the reverse complement of these miRNA may be engineered into a viral target sequence to produce a vaccine of the present invention.

TABLE 4
miRNA in the brain

| | SEQ | | SEQ |
|---|---|---|---|
| 5′ to 3′ miRNA sequence | ID | Reverse Complement (miRNA site) | ID |
| AAGUUUCUCUGAAUGUGUAGA | 3845 | UCUACACAUUCAGAGAAACUU | 3846 |
| AAUAUACAGGGGGAGACUCUUAU | 3847 | AUAAGAGUCUCCCCCUGUAUAUU | 3848 |
| AAUCAUUCACGGACAACACUUU | 3849 | AAAGUGUUGUCCGUGAAUGAUU | 3850 |
| AAUCUGAGAAGGCGCACAAGGUU | 3851 | AAACCUUGUGCGCCUUCUCAGAU | 3852 |
| U | | U | |
| AAUGUGUAGCAAAAGACAGA | 3853 | UCUGUCUUUUGCUACACAUU | 3854 |
| AAUGUGUAGCAAAAGACAGAAU | 3855 | AUUCUGUCUUUUGCUACACAUU | 3856 |
| ACCUUGGCUCUAGACUGCUUACU | 3857 | AGUAAGCAGUCUAGAGCCAAGGU | 3858 |
| ACUGGACUUGGAGUCAGAAG | 3859 | CUUCUGACUCCAAGUCCAGU | 3860 |
| AGAGGUUUUCUGGGUUUCUGUUU | 3861 | AAACAGAAACCCAGAAAACCUCU | 3862 |
| AGGCAUUAGAUUCUCAUUAGGA | 3863 | UCCUAAUGAGAAUCUAAUGCCU | 3864 |
| AGGGACUUUUGGGGGCAGAUGUG | 3865 | ACACAUCUGCCCCCAAAAGUCCC | 3866 |
| U | | U | |
| AGUUGGUCCGAGUGUUGUGGGUU | 3867 | AAUAACCCACAACACUCGGACCA | 3868 |
| AUU | | ACU | |
| AUAGGACUCAUAUAGUGCCA | 3869 | UGGCACUAUAUGAGUCCUAU | 3870 |
| AUAUACAGGGGGAGACUCUUAU | 3871 | AUAAGAGUCUCCCCCUGUAUAU | 3872 |
| AUCAUACAAGGACAAUUUCUUU | 3873 | AAAGAAAUUGUCCUUGUAUGAU | 3874 |
| AUCCCCAGAUACAAUGGACAAU | 3875 | AUUGUCCAUUGUAUCUGGGGAU | 3876 |
| CAACAAAUCACAGCCGGCCUCA | 3877 | UGAGGCCGGCUGUGAUUUGUUG | 3878 |
| CAGGCAGUGACUGUUCAGACGUC | 3879 | GACGUCUGAACAGUCACUGCCUG | 3880 |
| CCCCCCACUGCUAAAUUUGACUG | 3881 | AAGCCAGUCAAAUUUAGCAGUGG | 3882 |
| GCUU | | GGGG | |
| CUGUGGUUCCUGUAUGAAGACA | 3883 | UGUCUUCAUACAGGAACCACAG | 3884 |
| GAGAGAUCAGAGGCGCAGAGU | 3885 | ACUCUGCGCCUCUGAUCUCUC | 3886 |
| GCAUUGGUGGUUCAGUGGUAGAA | 3887 | GAAUUCUACCACUGAACCACCAA | 3888 |
| UUC | | UGC | |
| GCGUUGGUGGUAUAGUGG | 3889 | CCACUAUACCACCAACGC | 3890 |
| GCUCUGACUUUAUUGCACUACU | 3891 | AGUAGUGCAAUAAAGUCAGAGC | 3892 |
| GGAGACUGAUGAGUUCCCGGGA | 3893 | UCCCGGGAACUCAUCAGUCUCC | 3894 |
| GGAGGAACCUUGGAGCUUCGGCA | 3895 | UGCCGAAGCUCCAAGGUUCCUCC | 3896 |
| GGGGGCCGAUACACUGUACGAGA | 3897 | UCUCGUACAGUGUAUCGGCCCCC | 3898 |
| GUAAUGGUUAGCACUCUGG | 3899 | CCAGAGUGCUAACCAUUAC | 3900 |
| GUCUCUGUGGCGCAAUCGGU | 3901 | ACCGAUUGCGCCACAGAGAC | 3902 |
| UGAGUCUGUAAGAAAAGAGGAG | 3903 | CUCCUCUUUUCUUACAGACUCA | 3904 |
| UGGGCUGUAGUGCGCUAUGCC | 3905 | GGCAUAGCGCACUACAGCCCA | 3906 |
| UGGGCUGUAGUGCGCUAUGCCGA | 3907 | AUCGGCAUAGCGCACUACAGCCC | 3908 |
| U | | A | |
| UGGUCGACCAGUUGGAAAGUAAU | 3909 | AUUACUUUCCAACUGGUCGACCA | 3910 |
| UGGUCGACCAGUUGGAAAGUAAU | 3911 | AUUACUUUCCAACUGGUCGACCA | 3912 |
| UGUAGGGAUGGAAGCCAUGA | 3913 | UCAUGGCUUCCAUCCCUACA | 3914 |
| UGUAGGGAUGGAAGCCAUGAAA | 3915 | UUUCAUGGCUUCCAUCCCUACA | 3916 |

In one embodiment the presence of the virus in cells or tissues may be determined by looking for a “signature” of the virus. This signature may then inform the location of the virus and hence inform the selection of a miRNA binding site of an endogenous miRNA known to be expressed in that cellular location. The cellular environment in which a virus is present or has been present may be identified by its miRNA signature such as is described in US Publication 2011/0151430 to Kowalik and Stadler, the contents of which are incorporated herein by reference in its entirety. In a further embodiment of this aspect, the miRNAs may include any of the miRNAs of the eukaryotic miRNome.

According to the present invention, miRNA which are present in certain cells, tissues or environments may provide the sequence upon which to base the incorporated miRNA site engineered into the viral target sequences of the invention. Certain miRNA are known to be found in particular tissues or cells and representative examples are listed in Table 5.

TABLE 5
miRNA expression location

Dendritic Cells
let-7i
miR-142-3p
miR-146a
miR-148
miR-155
miR-221
miR-222
miRNA in Brain
mir-128
mir-219
mir-124a
mir-9
mir-135
mir-153
mir-183
miRNA in retinal epithelial cells
let-7b
let-7a
mir-125b
mir-24
mir-320
mir-23b
let-7e
let-7d
mir-23a
let-7c

### Antibiotics

The present invention may also be exploited to produce vaccines against bacterial infections. To this end, bacterial genomes, genes or sequences may be engineered to contain one or more miRNA sites. In one embodiment, targeted bacteria include both Gram negative and Gram positive bacteria. Examples of Gram positive bacteria include, but are not limited to Pasteurella species, Staphylococci species, and Streptococcus species. Examples of Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria spp. (e.g., M. tuberculosis, M. avium, M. intracellulare, M. kansasii, M. gordonae, M. leprae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter spp., Enterococcus spp., Haemophilus influenzae (Hemophilus influenza B, and Hemophilus influenza non-typable), Bacillus anthracis, Corynebacterium diphtheriae, Corynebacterium spp., Erysipelothrix rhusiopathiae, Clostridium perfringens, Clostridium tetani, Enterobacter aerogenes, Klebsiella pneumoniae, Pasturella multocida, Bacteroides spp., Fusobacterium nucleatum, Streptobacillus moniliformis, Treponema pallidum, Treponema pertenue, Leptospira, Rickettsia, Actinomyces israelii, meningococcus, pertussis, pneumococcus, shigella, tetanus, Vibrio cholerae, yersinia, Pseudomonas species, Clostridia species, Salmonella typhi, Shigella dysenteriae, Yersinia pestis, Brucella species, Legionella pneumophila, Rickettsiae, Chlamydia, Clostridium perfringens, Clostridium botulinum, Staphylococcus aureus, Pseudomonas aeruginosa, Cryptosporidium parvum, Streptococcus pneumoniae, and Bordetella pertussis.

### Amino Acid Based Vaccines

The vaccines of the present invention may also be polypeptide based molecules. In this embodiment, miRNA sites may be engineered into polynucleotides that encode one or more proteins from the pathogen. It is also within the scope of the invention for amino acid based vaccines to comprise one or more encoded proteins of the virus strain whereby no miRNA binding site is present. In this embodiment, replication would be a priori compromised as not all of the genes for replication would be present.

Chimeric nucleic acid/amino acid molecules are also contemplated such that the miRNA site is bound or linked to the polypeptide based vaccine. These molecules may be “peptides,” “polypeptides,” or “proteins.”

While it is known in the art that these terms imply relative size, these terms as used herein should not be considered limiting with respect to the size of the various polypeptide based molecules referred to herein and which are encompassed within this invention.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The amino acid sequences of the vaccines of the invention may comprise naturally occurring amino acids and as such may be considered to be proteins, peptides, polypeptides, or fragments thereof. Alternatively, the vaccines may comprise both naturally and non-naturally occurring amino acids.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions or deletions of amino acid residues that still maintain the properties of the parent polypeptide.

The term “derivative” is used synonymously with the term “variant” and refers to a molecule that has been modified or changed in any way relative to a reference molecule or starting molecule.

The present invention contemplates several types of vaccines which are amino acid based including variants and derivatives. These include substitutional, insertional, deletion and covalent variants and derivatives. As such, included within the scope of this invention are polypeptide based molecules containing substitutions, insertions and/or additions, deletions and covalently modifications. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins are those with one or more amino acids in the native or starting amino acid sequence removed. Ordinarily, deletional variants will have one or more amino acids deleted in a particular region of the molecule.

“Covalent derivatives” when referring to proteins include modifications of a native or starting protein with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present invention.

Other post-translational modifications include hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983).

Covalent derivatives specifically include fusion molecules in which proteins of the invention are covalently bonded to a non-proteinaceous polymer. The non-proteinaceous polymer ordinarily is a hydrophilic synthetic polymer, i.e. a polymer not otherwise found in nature. However, polymers which exist in nature and are produced by recombinant or in vitro methods are useful, as are polymers which are isolated from nature. Hydrophilic polyvinyl polymers fall within the scope of this invention, e.g. polyvinylalcohol and polyvinylpyrrolidone. Particularly useful are polyvinylalkylene ethers such a polyethylene glycol, polypropylene glycol. The proteins may be linked to various non-proteinaceous polymers, such as polyethylene glycol, polypropylene glycol or polyoxyalkylenes, in the manner set forth in U.S. Pat. No. 4,640,835; 4,496,689; 4,301,144; 4,670,417; 4,791,192 or 4,179,337.

“Features” when referring to proteins are defined as distinct amino acid sequence-based components of a molecule. Features of the proteins of the present invention include surface manifestations, local conformational shape, folds, loops, half-loops, domains, half-domains, sites, termini or any combination thereof.

As used herein when referring to proteins the term “surface manifestation” refers to a polypeptide based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol. Biol 266 (4): 814-830; 1997).

As used herein when referring to proteins the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/-0.5 amino acids). For example, a loop identified as a 7 amino acid loop could produce half-loops of 3 amino acids or 4 amino acids (7/2=3.5+/-0.5 being 3 or 4).

As used herein when referring to proteins the term “domain” refers to a motif of a polypeptide having one or more identifiable structural or functional characteristics or properties (e.g., binding capacity, serving as a site for protein-protein interactions.

As used herein when referring to proteins the term “half-domain” means portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/-0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid based embodiments is used synonymous with “amino acid residue” and “amino acid side chain.” A site represents a position within a peptide or polypeptide that may be modified, manipulated, altered, derivatized or varied within the polypeptide based molecules of the present invention.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a component of a molecule of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Delivery of Vaccines

The delivery of a vaccine to a subject in need thereof can be achieved in a number of different ways. In vivo delivery can be performed directly by administering a composition comprising a vaccine to a subject. Alternatively, delivery can be performed indirectly by administering one or more vectors that encode and direct the expression of the vaccine. These alternatives are discussed further below.

“Introducing into a cell,” when referring to a vaccine, means facilitating or effecting uptake or absorption into the cell, as is understood by those skilled in the art. Absorption or uptake of a vaccine can occur through unaided diffusive or active cellular processes, or by auxiliary agents or devices. The meaning of this term is not limited to cells in vitro; a vaccine may also be “introduced into a cell,” wherein the cell is part of a living organism. In such an instance, introduction into the cell will include the delivery to the organism. For example, for in vivo delivery, vaccines can be injected into a tissue site or administered systemically or intranasally. It is also contemplated by the inventors that introduction into cells or tissues may effected ex vivo, in situ and in ovo. In the case of transplants or within the field of stem cell technologies, it is contemplated that “introduction into a cell” will embrace the introduction to cells of any lineage or state, whether presently stem cells or which are intended to produce stem cells or progenitors or precursors thereof, as well as tissues, explants, organs and even organ systems.

### Direct Delivery

In general, any method of delivering a nucleic acid molecule can be adapted for use with a vaccine (see e.g., Akhtar S, and Julian R L. (1992) Trends Cell. Biol. 2(5):139-144 and WO94/02595, which are incorporated herein by reference in their entireties). However, there are three factors that are important to consider in order to successfully deliver a vaccine molecule in vivo: (a) biological stability of the delivered molecule, (2) preventing non-specific effects, and (3) accumulation of the delivered molecule in the target tissue. The non-specific effects of a vaccine can be minimized by local administration, for example by direct injection or implantation into a tissue (as a non-limiting example, a tumor) or topically administering the preparation.

For administering a vaccine systemically for the treatment of a disease, the vaccine can be modified or alternatively delivered using a drug delivery system; both methods act to prevent the rapid degradation of the molecule by endo- and exo-nucleases (in the case of nucleic acid based vaccines) in vivo. Modification of the RNA component of a vaccine or the pharmaceutical carrier can also permit targeting of the vaccine composition to the target tissue and avoid undesirable off-target effects. Vaccines modified by chemical conjugation to lipophilic groups such as cholesterol to enhance cellular uptake and prevent degradation. In like fashion, the vaccines of the present invention may be conjugated to one or more aptamers.

In an alternative embodiment, the vaccine can be delivered using drug delivery systems such as a nanoparticle, a dendrimer, a polymer, liposomes, or a cationic delivery system. Positively charged cationic delivery systems facilitate binding of a vaccine molecule (when negatively charged) and also enhance interactions at the negatively charged cell membrane to permit efficient uptake of a vaccine by the cell. Cationic lipids, dendrimers, or polymers can either be bound to a vaccine, or induced to form a vesicle or micelle that encases a vaccine. The formation of vesicles or micelles further prevents degradation of the vaccine when administered systemically. Methods for making and administering cationic-vaccine complexes are well within the abilities of one skilled in the art (see e.g., Sorensen, DR., et al (2003) J. Mol. Biol. 327:761-766; Verma, UN., et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of vaccines include DOTAP (Sorensen, DR., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, a vaccine forms a complex with cyclodextrin for systemic administration.

### Vector Encoded Vaccines

In another aspect, vaccines can be expressed from transcription units inserted into DNA or RNA vectors. Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

Expression vectors are generally DNA plasmids or viral vectors. Expression vectors compatible with eukaryotic cells, preferably those compatible with vertebrate cells, can be used to produce recombinant constructs for the expression of a vaccine as described herein. Eukaryotic cell expression vectors are well known in the art and are available from a number of commercial sources. Typically, such vectors are provided containing convenient restriction sites for insertion of the desired nucleic acid segment.

Delivery of vaccine expressing vectors can be systemic, such as by intravenous or intramuscular administration, by administration to target cells ex-planted from the patient followed by reintroduction into the patient, or by any other means that allows for introduction into a desired target cell.

Vaccine expression plasmids can be transfected into target cells as a complex with cationic lipid carriers (e.g., Oligofectamine) or non-cationic lipid-based carriers (e.g., Transit-TKO™). Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct may be incorporated into vectors capable of episomal replication, e.g EPV and EBV vectors. Constructs for the recombinant expression of a vaccine will generally require regulatory elements, e.g., promoters, enhancers, etc., to ensure the expression of the vaccine in target cells. Other aspects to consider for vectors and constructs are further described below.

Vectors useful for the delivery of a vaccine may include regulatory elements (promoter, enhancer, etc.) sufficient for expression of the vaccine in the desired target cell or tissue. The regulatory elements can be chosen to provide either constitutive or regulated/inducible expression.

Expression of the vaccine can be precisely regulated, for example, by using an inducible regulatory sequence that is sensitive to certain physiological regulators, e.g., circulating glucose levels, or hormones (Docherty et al., 1994, FASEB J. 8:20-24). Such inducible expression systems, suitable for the control of expression in cells or in mammals include, for example, regulation by ecdysone, by estrogen, progesterone, tetracycline, chemical inducers of dimerization, and isopropyl-beta-D1-thiogalactopyranoside (IPTG). A person skilled in the art would be able to choose the appropriate regulatory/promoter sequence based on the intended use of the transgene.

In a specific embodiment, viral vectors that contain nucleic acid sequences encoding a vaccine can be used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding a vaccine are cloned into one or more vectors, which facilitates delivery of the nucleic acid into a cell, tissue or patient. More detail about retroviral vectors can be found, for example, in Boesen et al., Biotherapy 6:291-302 (1994), which describes the use of a retroviral vector to deliver the mdr1 gene to hematopoietic stem cells in order to make the stem cells more resistant to chemotherapy.

In one embodiment, the vaccines of the present invention may be delivered via a bacterial delivery approach as disclosed in PCT Publication WO/2008/156702, the contents of which are incorporated herein in its entirety.

Adenoviruses are also contemplated for use in delivery of nucleic acid based vaccines. Adenoviruses are especially attractive vehicles, e.g., for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys.

A suitable AV vector for expressing a vaccine featured in the invention, a method for constructing the recombinant AV vector, and a method for delivering the vector into target cells, are described in Xia H et al. (2002), Nat. Biotech. 20: 1006-1010. Use of Adeno-associated virus (AAV) vectors is also contemplated (Walsh et al., Proc. Soc. Exp. Biol. Med. 204:289-300 (1993); U.S. Pat. No. 5,436,146).

In one embodiment, the vaccine can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the vaccines featured in the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

Another preferred viral vector is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, and the like. AAV vectors can be made to target different cells by engineering the vectors to express different capsid protein serotypes; see, e.g., Rabinowitz J E et al. (2002), J Virol 76:791-801, the entire disclosure of which is herein incorporated by reference.

The pharmaceutical preparation of a vector can include the vector in an acceptable diluent or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Formulations

In one embodiment, a vaccine featured in the invention is fully encapsulated in a lipid formulation, e.g., to form a SPLP, pSPLP, SNALP, or other nucleic acid-lipid particle. As used herein, the term “SNALP” refers to a stable nucleic acid-lipid particle, including SPLP. SNALPs are described, e.g., in U.S. Patent Application Publication Nos. 20060240093, 20070135372, and in International Application No. WO 2009082817. These applications are incorporated herein by reference in their entirety. In one embodiment, lipids and/or lipid-containing compositions or formulations described herein are used as adjuvants when delivered with the vaccines of the present invention. As used herein, an “adjuvant” is any agent that modifies the effect of another agent. In the present case, the lipids or lipid-based formulations may function to alter the effect of the vaccine on the subject, e.g., improving the immune response elicited.

As used herein, the term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection and accumulate at distal sites (e.g., sites physically separated from the administration site). SPLPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964, each of which is incorporated herein by reference in its entirety.

In one embodiment, the lipid to drug ratio (mass/mass ratio) (e.g., lipid to vaccine ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1.

The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA),1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA),1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl-4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1), or a mixture thereof. The cationic lipid may comprise from about 20 mol % to about 50 mol % or about 40 mol % of the total lipid present in the particle.

In another embodiment, the compound 2,2-Dilinoleyl-4-dimethylaminoethyl-[1,3]-dioxolane can be used to prepare lipid nanoparticles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

In one embodiment, the particle includes 40% 2,2-Dilinoleyl-4-dimethylaminoethyl-[1,3]-dioxolane:10% DSPC:40% Cholesterol:10% PEG-C-DOMG (mole percent) with a particle size of 63.0±20 nm.

The non-cationic lipid may be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE),16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid may be from about 5 mol % to about 90 mol %, about 10 mol %, or about 58 mol % if cholesterol is included, of the total lipid present in the particle.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles may be from 0 mol % to about 20 mol % or about 2 mol % of the total lipid present in the particle.

In some embodiments, the nucleic acid-lipid particle further includes cholesterol at, e.g., about 10 mol % to about 60 mol % or about 48 mol % of the total lipid present in the particle.

In one embodiment, the lipidoid ND98•4HCl (MW 1487) (see U.S. patent application Ser. No. 12/056,230, filed Mar. 26, 2008, which is herein incorporated by reference), Cholesterol (Sigma-Aldrich), and PEG-Ceramide C16 (Avanti Polar Lipids) can be used to prepare nanoparticles (i.e., LNP01 particles). Stock solutions of each in ethanol can be prepared as follows: ND98, 133 mg/ml; Cholesterol, 25 mg/ml, PEG-Ceramide C16, 100 mg/ml. The ND98, Cholesterol, and PEG-Ceramide C16 stock solutions can then be combined in a, e.g., 42:48:10 molar ratio. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4. LNP01 formulations are described, e.g., in International Application Publication No. WO 2008/042973, which is hereby incorporated by reference. Additional exemplary lipid formulations are shown in Table 6.

TABLE 6
Lipid Nanoparticle formulations

| | | cationic lipid/non-cationic |
|---|---|---|
| | | lipid/cholesterol/PEG-lipid conjugate |
| | | Lipid:nucleic acid (e.g., nucleic acid or |
| | Cationic Lipid | vaccine) ratio |
| SNALP | l,2-Dilinolenyloxy-N,N- | DLinDMA/DPPC/Cholesterol/PEG-cDMA |
| | dimethylaminopropane | (57.1/7.1/34.4/1.4) |
| | (DLinDMA) | lipid:vaccine ~7:1 |
| S-XTC | 2,2-Dilinoleyl-4- | XTC/DPPC/Cholesterol/PEG-cDMA |
| | dimethylaminoethyl-[1,3]- | 57.1/7.1/34.4/1.4 |
| | dioxolane (XTC) | lipid:vaccine ~7:1 |
| LNP05 | 2,2-Dilinoleyl-4- | XTC/DSPC/Cholesterol/PEG-DMG |
| | dimethylaminoethyl-[1,3]- | 57.5/7.5/31.5/3.5 |
| | dioxolane (XTC) | lipid:vaccine ~6:1 |
| LNP06 | 2,2-Dilinoleyl-4- | XTC/DSPC/Cholesterol/PEG-DMG |
| | dimethylaminoethyl-[1,3]- | 57.5/7.5/31.5/3.5 |
| | dioxolane (XTC) | lipid:vaccine ~11:1 |
| LNP07 | 2,2-Dilinoleyl-4- | XTC/DSPC/Cholesterol/PEG-DMG |
| | dimethylaminoethyl-[1,3]- | 60/7.5/31/1.5, |
| | dioxolane (XTC) | lipid:vaccine ~6:1 |
| LNP08 | 2,2-Dilinoleyl-4- | XTC/DSPC/Cholesterol/PEG-DMG |
| | dimethylaminoethyl-[1,3]- | 60/7.5/31/1.5, |
| | dioxolane (XTC) | lipid:vaccine ~11:1 |
| LNP09 | 2,2-Dilinoleyl-4- | XTC/DSPC/Cholesterol/PEG-DMG |
| | dimethylaminoethyl-[1,3]- | 50/10/38.5/1.5 |
| | dioxolane (XTC) | Lipid:vaccine 10:1 |
| LNP10 | (3aR,5s,6aS)-N,N- | ALN100/DSPC/Cholesterol/PEG-DMG |
| | dimethyl-2,2-di((9Z,12Z)- | 50/10/38.5/1.5 |
| | octadeca-9,12- | Lipid:vaccine 10:1 |
| | dienyl)tetrahydro-3aH- | |
| | cyclopenta[d][1,3]dioxol- | |
| | 5-amine (ALN100) | |
| LNP11 | (6Z,9Z,28Z,31Z)- | MC-3/DSPC/Cholesterol/PEG-DMG |
| | heptatriaconta-6,9,28,31- | 50/10/38.5/1.5 |
| | tetraen-19-yl 4- | Lipid:vaccine 10:1 |
| | (dimethylamino)butanoate | |
| | (MC3) | |
| LNP12 | 1,1′-(2-(4-(2-((2-(bis(2- | C12-200/DSPC/Cholesterol/PEG-DMG |
| | hydroxydodecyl)amino)ethyl)(2- | 50/10/38.5/1.5 |
| | hydroxydodecyl)amino)ethyl) | Lipid:vaccine 10:1 |
| | piperazin-1- | |
| | yl)ethylazanediyl)didodecan- | |
| | 2-ol (C12-200) | |
| LNP13 | XTC | XTC/DSPC/Chol/PEG-DMG |
| | | 50/10/38.5/1.5 |
| | | Lipid:vaccine: 33:1 |
| LNP14 | MC3 | MC3/DSPC/Chol/PEG-DMG |
| | | 40/15/40/5 |
| | | Lipid:vaccine: 11:1 |
| LNP15 | MC3 | MC3/DSPC/Chol/PEG-DSG/GalNAc-PEG-DSG |
| | | 50/10/35/4.5/0.5 |
| | | Lipid:vaccine: 11:1 |
| LNP16 | MC3 | MC3/DSPC/Chol/PEG-DMG |
| | | 50/10/38.5/1.5 |
| | | Lipid:vaccine: 7:1 |
| LNP17 | MC3 | MC3/DSPC/Chol/PEG-DSG |
| | | 50/10/38.5/1.5 |
| | | Lipid:vaccine: 10:1 |
| LNP18 | MC3 | MC3/DSPC/Chol/PEG-DMG |
| | | 50/10/38.5/1.5 |
| | | Lipid:vaccine: 12:1 |
| LNP19 | MC3 | MC3/DSPC/Chol/PEG-DMG |
| | | 50/10/35/5 |
| | | Lipid:vaccine: 8:1 |
| LNP20 | MC3 | MC3/DSPC/Chol/PEG-DPG |
| | | 50/10/38.5/1.5 |
| | | Lipid:vaccine: 10:1 |
| LNP21 | C12-200 | C12-200/DSPC/Chol/PEG-DSG |
| | | 50/10/38.5/1.5 |
| | | Lipid:vaccine: 7:1 |
| LNP22 | XTC | XTC/DSPC/Chol/PEG-DSG |
| | | 50/10/38.5/1.5 |
| | | Lipid:vaccine: 10:1 |

DSPC: distearoylphosphatidylcholine

DPPC: dipalmitoylphosphatidylcholine

PEG-DMG: PEG-didimyristoyl glycerol (C14-PEG, or PEG-C14) (PEG with avg mol wt of 2000)

PEG-DSG: PEG-distyryl glycerol (C18-PEG, or PEG-C18) (PEG with avg mol wt of 2000)

PEG-cDMA: PEG-carbamoyl-1,2-dimyristyloxypropylamine (PEG with avg mol wt of 2000)

SNALP (1,2-Dilinolenyloxy-N,N-dimethylaminopropane (DLinDMA)) comprising formulations are described in International Publication No. WO2009/127060, filed Apr. 15, 2009, which is hereby incorporated by reference in its entirety.

XTC comprising formulations are described, e.g., in U.S. Provisional Ser. No. 61/239,686, filed Sep. 3, 2009 as well as PCT/US10/22614 filed Jan. 29, 2010 each of which is hereby incorporated by reference in its entirety. Further XTC formulations useful in the present invention are disclosed in PCT/US08/088,588 filed 31 Dec. 2008 and PCT/US08/88587 filed 31 Dec. 2008 and PCT/US09/041,442 filed 22 Apr. 2009 and PCT/US09/061,897 filed 23 Oct. 2009 and PCT/US10/38224 filed Jun. 10, 2010, each of which is hereby incorporated by reference in its entirety.

MC3 comprising formulations are described, e.g., in U.S. Provisional Ser. No. 61/244,834, filed Sep. 22, 2009, and U.S. Provisional Ser. No. 61/185,800, filed Jun. 10, 2009, and PCT/US09/63933 filed Nov. 10, 2009 and PCT/US09/63927 filed 10 Nov. 2009 and PCT/US09/63931 filed 10 Nov. 2009 and PCT/US09/63897 filed 10 Nov. 2009, each of which are hereby incorporated by reference in its entirety.

ALNY-100 comprising formulations are described, e.g., International patent application number PCT/US09/63933, filed on Nov. 10, 2009, which is hereby incorporated by reference in its entirety.

C12-200 comprising formulations are described in U.S. Provisional Ser. No. 61/175,770, filed May 5, 2009, as well as PCT/US10/33777 which are hereby incorporated by reference in its entirety.

Transfection reagents useful in the present invention are disclosed in U.S. provisional 61/267,419 filed Dec. 7, 2009, which is hereby incorporated by reference in its entirety.

Formulations for targeting immune cells useful in the present invention are disclosed in PCT/US10/033,747 filed May 5, 2010, which is hereby incorporated by reference in its entirety.

Pyrrolidine cationic lipids useful in the formulations of the present invention are disclosed in U.S. Ser. No. 12/123,922 filed May 20, 2008 which is hereby incorporated by reference in its entirety.

In one embodiment, the reagent that facilitates targeting construct uptake used herein comprises a cationic lipid as described in e.g., U.S. Application Ser. No. 61/267,419, filed 7 Dec. 2009, and U.S. Application Ser. No. 61/334,398, filed 13 May 2010. In various embodiments, the composition described herein comprises a cationic lipid selected from the group consisting of: “Lipid H”, “Lipid K”; “Lipid L”, “Lipid M”; “Lipid P”; or “Lipid R”, whose formulas are indicated as follows:

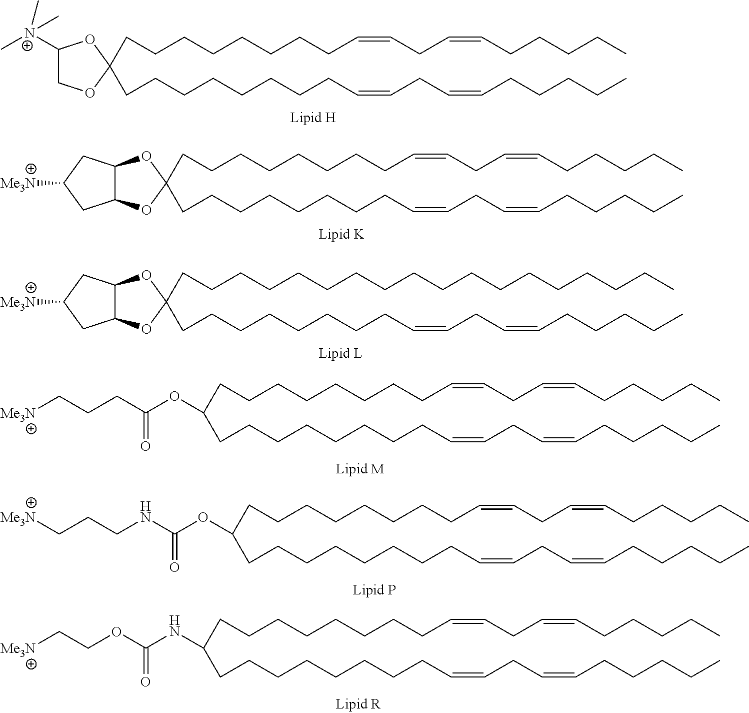

Also contemplated herein are various formulations of the lipids described above, such as, e.g., K8, P8 and L8 which refer to formulations comprising Lipid K, P, and L, respectively. Some exemplary lipid formulations for use with the methods and compositions described herein are found in Table 7.

TABLE 7
Example lipid formulations

| Formulation | Cationic Lipid | Cationic Lipid | DOPE | Cholesterol |
|---|---|---|---|---|
| Number | Number | Mol % | % | % |
| 1 | 200 (Lipid H) | 48.08 | 51.92 | — |
| 2 | 200 (Lipid H) | 47.94 | 47.06 | 5 |
| 3 | 201 (Lipid K) | 45.56 | 54.44 | — |
| 4 (K8) | 201 (Lipid K) | 47.94 | 47.06 | 5 |
| 5 (L8) | 202 (Lipid L) | 47.94 | 47.06 | 5 |
| 6 | 203 (Lipid M) | 53.01 | 44.49 | 2.5 |
| 7 | 203 (Lipid M) | 47.94 | 47.06 | 5 |
| 8 (P8) | 204 (Lipid P) | 47.94 | 47.06 | 5 |
| 9 | 205 (Lipid R) | 47.94 | 47.06 | 5 |

In another embodiment, the composition described herein further comprises a lipid formulation comprising a lipid selected from the group consisting of Lipid H, Lipid K, Lipid L, Lipid M, Lipid P, and Lipid R, and further comprises a neutral lipid and a sterol. In particular embodiments, the lipid formulation comprises between approximately 25 mol %-100 mol % of the lipid. In another embodiment, the lipid formulation comprises between 0 mol %-50 mol % cholesterol. In still another embodiment, the lipid formulation comprises between 30 mol %-65 mol % of a neutral lipid. In particular embodiments, the lipid formulation comprises the relative mol % of the components as listed in Table 8 as follows:

TABLE 8
Example lipid formulae

| Series | Lipid (Mol %) | DOPE | Chol |
|---|---|---|---|
| 1 | 45.56 | 54.44 | 0 |
| 2 | 48.08 | 51.92 | 0 |
| 3 | 50.60 | 49.40 | 0 |
| 4 | 53.10 | 46.90 | 0 |
| 5 | 52.73 | 37.27 | 10 |
| 6 | 52.92 | 42.08 | 5 |
| 7 | 53.01 | 44.49 | 2.5 |
| 8 | 47.94 | 47.06 | 5 |

### Other Particles

In vivo delivery can also be by a beta-glucan delivery system, such as those described in U.S. Pat. Nos. 5,032,401 and 5,607,677, and U.S. Publication No. 2005/0281781, which are hereby incorporated by reference in their entirety. In vitro introduction into a cell includes methods known in the art such as electroporation and lipofection.

In one embodiment, core-shell nanoparticles may be used for delivery to cells, tissues or organ systems. Such core-shell nanoparticles are described by Siegwart (Siegwart, et al., Combinatorial synthesis of chemically diverse core-shell nanoparticles for intracellular delivery, PNAS, PNAS Early edition, Jul. 22, 2011; the contents of which are incorporated herein in their entirety) and comprise a cationic core to facilitate vaccine complexation, with variation in the nature of the protonizable amine, and a shell with variation in polymer length and chemical properties. Block copolymers created by reacting epoxide groups with amines and possessing poly(oligo(ethylene oxide) methacrylate) (POEOMA) with different lengths of the PEO side chain, may increase blood circulation time due to the PEO shell of the resulting nanoparticle. Anionic, cationic, zwitterionic, and hydrophobic blocks may also be used as shells.

### Liposomal Formulations

There are many organized surfactant structures that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to traverse intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively charged entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C1215G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Compositions and formulations for parenteral, intraparenchymal (into the brain), intrathecal, intraventricular or intrahepatic administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations of vaccines. These compositions may be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

### Emulsions

The compositions of the present invention may be prepared and formulated as emulsions. Emulsions are typically heterogeneous systems of one liquid dispersed in another in the form of droplets usually exceeding 0.1 um in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants may be classified into different classes based on the nature of the hydrophilic group: nonionic, anionic, cationic and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of vaccines are formulated as microemulsions. A microemulsion may be defined as a system of water, oil and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile vaccine drugs, or peptides. Microemulsions have also been effective in the transdermal delivery of active components in both cosmetic and pharmaceutical applications. It is expected that the microemulsion compositions and formulations of the present invention will facilitate the increased systemic absorption of nucleic acid based vaccines from the gastrointestinal tract, as well as improve the local cellular uptake.

Microemulsions of the present invention may also contain additional components and additives such as sorbitan monostearate (Grill 3), Labrasol, and penetration enhancers to improve the properties of the formulation and to enhance the absorption of the vaccines and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Penetration Enhancers

In one embodiment, the present invention employs various penetration enhancers to effect the efficient delivery of vaccines to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, N.Y., 2002; Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of vaccines through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, N.Y., 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, Mass., 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (see e.g., Malmsten, M. Surfactants and polymers in drug delivery, Informa Health Care, New York, N.Y., 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, N.Y., 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of vaccines through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, Mass., 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of vaccines through the alimentary mucosa (see e.g., Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of vaccines at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of nucleic acids. Examples of commercially available transfection reagents include, for example Lipofectamine™ (Invitrogen; Carlsbad, Calif.), Lipofectamine 2000™ (Invitrogen; Carlsbad, Calif.), 293Fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass^{a} D1 Transfection Reagent (New England Biolabs; Ipswich, Mass., USA), LyoVec™/LipoGen™ (Invivogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Other agents may be utilized to enhance the penetration of the administered nucleic acids, including glycols such as ethylene glycol and propylene glycol, pyrrols such as 2-pyrrol, azones, and terpenes such as limonene and menthone.

### Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor.

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with vaccines which are nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

### Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol and/or dextran. The suspension may also contain stabilizers.

In addition to their administration, as discussed above, the vaccines featured in the invention can be administered in combination with other known agents effective in treatment of pathological processes. In any event, the administering physician can adjust the amount and timing of administration on the basis of results observed using standard measures of efficacy known in the art or described herein.

Further, toxicity and therapeutic efficacy of compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Patient Populations

According to the present invention, the vaccines described herein may be used prophylactically or to treat or ameliorate disease. In one embodiment the vaccine composition is administered to an asymptomatic carrier of a disease (virus) to prevent the spread to others. In another embodiment the vaccine composition is administered prophylactically. In one embodiment the vaccine composition is administered after infection but before viral shedding. In this embodiment, infection can be determined by evaluating the pathogens miRNA signature or other means of detecting the presence of the pathogen (e.g., virus or viral sequences). In one embodiment, the vaccine composition is administered after viral shedding has begun and the subject is symptomatic. In another embodiment, the vaccine composition is administered days, weeks or months after an outbreak. In one embodiment, the vaccine composition is administered to non-infected individuals to prevent their future infection by the pathogen.

In one embodiment, the invention provides pharmaceutical compositions containing a vaccine composition, as described herein, and a pharmaceutically acceptable carrier. Such pharmaceutical compositions are formulated based on the mode of delivery. One example is compositions that are formulated for systemic administration via parenteral delivery, e.g., by intravenous (IV) delivery. Another example is compositions that are formulated for direct delivery into the brain parenchyma, e.g., by infusion into the brain, such as by continuous pump infusion.

The pharmaceutical compositions featured herein are administered in dosages sufficient to trigger an immune response. In general, a suitable dose will be in the range of 0.01 to 200.0 milligrams per kilogram body weight of the recipient per day, generally in the range of 1 to 50 mg per kilogram body weight per day. For example, the vaccine can be administered at 0.05 mg/kg, 0.5 mg/kg, 1 mg/kg, 1.5 mg/kg, 2 mg/kg, 3 mg/kg, 10 mg/kg, 20 mg/kg, 30 mg/kg, 40 mg/kg, or 50 mg/kg per single dose. The pharmaceutical composition may be administered once daily or it may be administered as two, three, or more sub-doses at appropriate intervals throughout the day or even using continuous infusion or delivery through a controlled release formulation. In that case, the vaccine contained in each sub-dose must be correspondingly smaller in order to achieve the total daily dosage. The dosage unit can also be compounded for delivery over several days, e.g., using a conventional sustained release formulation which provides sustained release over a several day period. Sustained release formulations are well known in the art and are particularly useful for delivery of agents at a particular site, such as could be used with the agents of the present invention. In this embodiment, the dosage unit contains a corresponding multiple of the daily dose.

The effect of a single dose can be long lasting, such that subsequent doses are administered at not more than 3, 4, or 5 day intervals, or at not more than 1, 2, 3, or 4 week intervals. It is also understood that the compositions of the present invention may be administered on a monthly, yearly, or long-term repeated schedule as is typical with immunization or “booster” schedules. To this end the compositions may be administered every 6 months, every year, every 2 years, every 5 years or every 10 years, or more.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual vaccine composition encompassed by the invention can be made using conventional methodologies or on the basis of in vivo testing using an appropriate animal model.

### Kits

Any of the compositions described herein may be comprised in a kit. The kit may further include reagents or instructions for creating or synthesizing the vaccines. It may also include one or more buffers, such as a nuclease buffer, transcription buffer, or a hybridization buffer, compounds for preparing the DNA template or a dsRNA, and components for isolating the resultant template, target sequence or vaccine. Other kits of the invention may include components for making a nucleic acid array and thus, may include, for example, a solid support.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the vaccine, e.g., nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the invention. The dye may then be resuspended in any suitable solvent, such as DMSO.

The container means will generally include at least one vial, test tube, flask, bottle, syringe and/or other container means, into which the vaccine, e.g., nucleic acid formulations are placed, preferably, suitably allocated. The kits may also comprise a second container means for containing a sterile, pharmaceutically acceptable buffer and/or other diluent.

The kits of the present invention may also typically include a means for containing the vials in close confinement for commercial sale, such as, e.g., injection and/or blow-molded plastic containers into which the desired vials are retained.

Kits may also include components that facilitate isolation of a DNA template. It may also include components that preserve or maintain the nucleic acids or that protect against their degradation. Such components may be RNAse-free or protect against RNAses, such as RNase inhibitors. Such kits generally will comprise, in suitable means, distinct containers for each individual reagent or solution.

A kit can include instructions for employing the kit components as well the use of any other reagent not included in the kit. Instructions may include variations that can be implemented.

### EXAMPLES

### Example 1

### Viral Attenuation Reporter System

A dual luciferase reporter system was designed to assess the efficacy of the vaccines of the present invention. In this system, attenuation is determined by monitoring luminescence of the firefly luciferase normalized to the luminescence of the renilla luciferase. Each viral gene of interest, containing one or more miRNA target sites (or mutant versions as controls), are cloned upstream of firefly luciferase gene. Constructs are expressed in a variety of mammalian cell lines and luciferase activity is measured. Successful attenuation is measured as a decrease in luciferase activity as compared to cells that are not expressing the relevant miRNA.

### Example 2

### Plaque Assay

Screening of the modified viruses may be performed by a plaque assay. When partial or complete viral genomes are modified by insertion of one or more miRNA target sites, modified viruses are screened via a plaque assay. A cell line susceptible to lytic infection is plated as a lawn. Viral supernatants generated from cells infected with modified genomes are added to the lawns at known dilutions. After incubation, cells are fixed, stained, and lytic plaques formed in the lawn are counted for back calculation of the sample's viral titer. Typically, the cell line used in the assay is a mammalian cell line, such as a rodent, non-human primate (e.g., monkey), or human cell line. Cell lines used in the invention may include Vero, MRC-5, BHK, CEM, and LL-1 cells. Relevant cell types for HSV viral replication include, but are not limited to, epithelial cells, and monocyte/dendritic cells.

A model viral genome with a modification for ease of measuring viral titer may also be employed. For instance, a viral genome encoding a GFP-fusion protein that would be packaged with the virus may serve as a beacon for measurement. Viral count may be tied to the total fluorescence measured in the supernatant via fluorimeter or spectrophotometer. Additionally, viral fluorescence of a sample may be obtained by capture of viruses on a fixed substrate such as a well in a plate or latex bead to assist with measuring. Captured viruses' fluorescence may be measured using flow cytometry or other similar methods. Viral titers could be calculated comparing a standard curve of the GFP-containing viral strain whose fluorescence in supernatants has been correlated with the plaque assay.

### Example 3

### Design of miRNA Binding Sites within HSV Genes

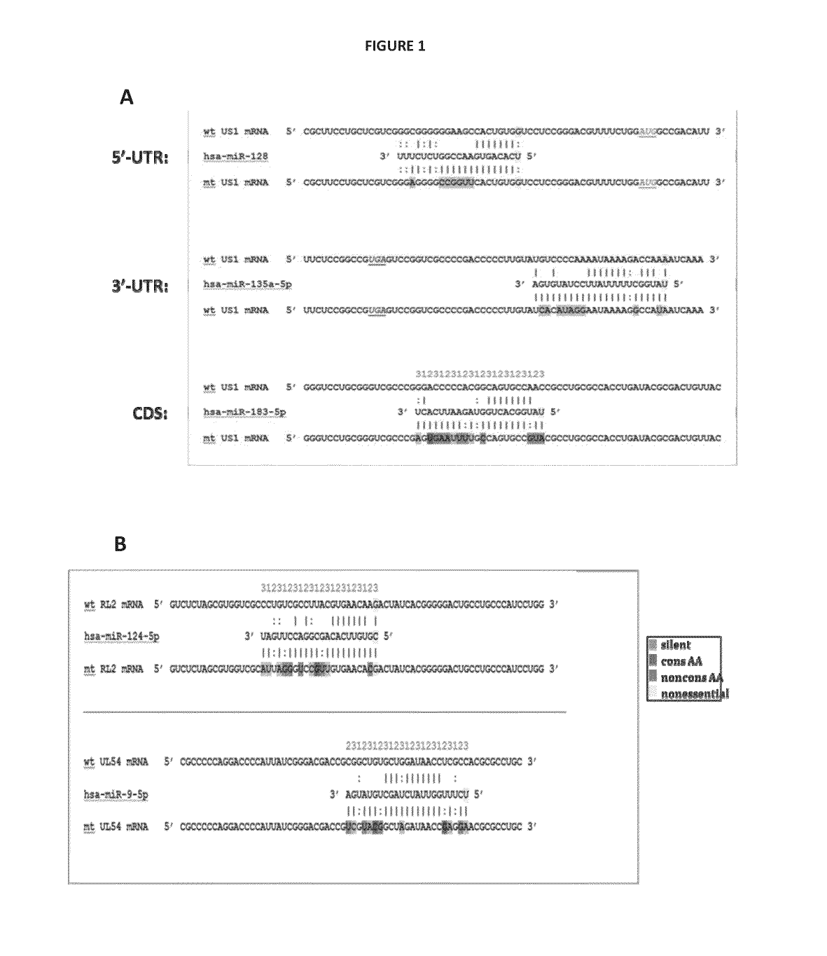

miRNA binding sites were engineered into either the US1 (FIG. 1A) or RL2 (FIG. 1B) genes.

Candidate HSV1 gene mRNA sequences, including US1, US10, US11, US12, RL2, and UL54, were individually aligned in the plus/minus orientation with each of the human mature miR-128, miR-219, miR-124a, miR-9, miR-135, miR-153, and miR-183 sequences via pairwise BLASTN (http://blast.ncbi.nlm.nih.gov/). Candidate mRNA /miRNA pairs that had high-scoring matches including the miRNA seed region were saved, and re-aligned manually. Next, candidate mutations were introduced to the miRNA sequence to maximize target mRNA/miRNA complementarity while minimizing alteration of target gene function (FIG. 1). Watson-Crick pairs were favored over non-canonical (“wobble”) G:U pairs. For target gene 5′- and 3′-UTR regions, all nucleotides (at each position) were considered equally functional, so engineering perfect mRNA/miRNA complementarity was straightforward. For target gene coding sequences (“CDS”), candidate mutations that minimized alteration of the encoded protein were favored: Silent mutations that do not alter the encoded amino acid, over Conservative mutations that cause an amino acid to be replaced with another amino acid bearing very similar side-chain physicochemical characteristics (e.g. Small AND Polar, Polar AND Positive, Hydrophobic AND Aromatic), over Semiconservative mutations that cause an amino acid to be replaced with another amino acid bearing similar side-chain physical characteristics (e.g. Small, Polar, Hydrophobic). Radical replacements and nonsense mutations were not considered, on the grounds that they would be maximally disruptive to target gene (protein) function.

### Example 4

### Detection and Quantitation of HSV

Total viral particles in the supernatant of cultures of infected cells is quantified by measuring the concentration of viral genomic DNA by qPCR. At the desired time point, infected cell supernatants are removed from the 96 well tissue culture plates. Viral DNA is isolated from 50u1 of the supernatant using Magmax Viral RNA Isolation Kit (Applied Biosystems, AM-1836) following the protocol as per kit instructions. Real time PCR (qPCR) is performed using 3-4 ul of obtained cDNA using a Roche LightCycler 480. Reagents used for this reaction include: Roche LightCycler PCR Master Mix and pathogen detection primer/probe kit from Primer Design Ltd for HSV 1 or 2 (Path-HSV1-std) or (Path-HSV2-std), respectively. Standard curves are generated for each qPCR reaction using the corresponding HSV strain standard obtained with the primer/probe kit from Primer Design Ltd. Six 1:10 dilutions of the standard are used to generate the standard curve from which the viral genome numbers were quantified.

Extraction of HSV DNA is performed generally by the methods of Namvar, et al. (J Clin Microbiol. 2005 May; 43(5): 2058-2064). Briefly, DNA is extracted in a Magnapure LC robot (Roche Diagnostics, Mannheim, Germany) using the Magnapure DNA Isolation Kit according to the manufacturer's instructions. The input and output volumes are set to 200 μl and 100 μl, respectively. Freeze-thawing of the sample may be used as an alternative method for DNA preparation. In these cases 10 μl of the thawed sample is used in PCR without further procedures.

1. A mutant HSV-1 strain comprising at least one miRNA site.

2. The mutant HSV-1 strain of claim 1, wherein the miRNA site is present in an untranslated region of an HSV-1 gene encoded by the HSV-1 strain.

3. The mutant HSV-1 strain of claim 2, wherein the untranslated region is selected from the group consisting of the 3′UTR, the 5′ UTR, an intron, and an intragenic region.

4. The mutant HSV-1 strain of claim 3, wherein the at least one miRNA site is selected from the miRNA sites of Table 3.

5. The mutant HSV-1 strain of claim 4, wherein the miRNA site is 17-25 nucleotides in length.

6. The mutant HSV-1 strain of claim 5, further comprising a second miRNA site.

7. The mutant HSV-1 strain of claim 6, wherein said second miRNA site has the same nucleotide sequence as the at least one miRNA site.

8. The mutant HSV-1 strain of claim 6, wherein said second miRNA site is different from the at least one miRNA site.

9. The mutant HSV-1 strain of claim 6 further comprising three or more miRNA sites.

10. The mutant HSV-1 strain of claim 1, wherein the miRNA site is present in a coding region of an HSV-1 gene encoded by the HSV-1 strain.

11. The mutant HSV-1 strain of claim 10, wherein the gene comprising the miRNA site is inactivated, thereby producing an attenuated HSV-1 virus.

12. A vaccine comprising the mutant HSV-1 strain of claim 1.

13. A method of immunizing a subject with an HSV-1 antigen comprising contacting said subject with a composition comprising a mutant HSV-1 strain, mutant HSV-1 gene or mutant HSV-1 polynucleotide sequence, wherein the mutant strain, gene or polynucleotide sequence has been engineered to contain at least one miRNA site of Table 3.

14. The method of claim 13, wherein the subject is contacted more than once.

15. The method of claim 14, wherein the subject is contacted yearly, every 2 years or every 5 years.

16. The method of claim 13, wherein composition is formulated for systemic delivery.

17. The method of claim 16, wherein systemic delivery is by intravenous or intramuscular administration.

18. The method of claim 13, wherein the composition further comprises one or more adjuvants.

19. The method of claim 18, wherein the adjuvant is a lipid or lipid-based agent.

20. The method of claim 19, wherein the lipid is a cationic lipid.
